SEQUENCE LISTING (134 sequences)

SEQ ID NO: 1 - protein, 10 aa, Artificial
  CDRH1
GDSVSSNNAV

SEQ ID NO: 2 - protein, 9 aa, Artificial
  CDRH2
TYYRSKWYN

SEQ ID NO: 3 - protein, 18 aa, Artificial
  CDRH3
VRSGHITVFGVNVDAFDM

SEQ ID NO: 4 - protein, 8 aa, Artificial
  CDRL1
QSLSSYLH

SEQ ID NO: 5 - protein, 3 aa, Artificial
  CDRL2
AAS

SEQ ID NO: 6 - protein, 5 aa, Artificial
  CDRL3
QQSRT

SEQ ID NO: 7 - protein, 128 aa, Artificial
  VH
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSS

SEQ ID NO: 8 - protein, 103 aa, Artificial
  VL
DIQMTQSPSSLSASVGDRVTITCRTSQSLSSYLHWYQQKPGKAPKLLIYAASSLQSGVPSRFSGSGSGTDFTLTISSLQPEDFATYYCQQSRTFGQGTKVEIK

SEQ ID NO: 9 - protein, 458 aa, Artificial
  Heavy chain
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 210 aa, Artificial
  Light chain
DIQMTQSPSSLSASVGDRVTITCRTSQSLSSYLHWYQQKPGKAPKLLIYAASSLQSGVPSRFSGSGSGTDFTLTISSLQPEDFATYYCQQSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 4 aa, Artificial
  CDRL2 long
AASS

SEQ ID NO: 12 - protein, 458 aa, Artificial
  Heavy chain
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 458 aa, Artificial
  Heavy chain
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 14 - protein, 406 aa, Artificial
  Heavy chain
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTP

SEQ ID NO: 15 - protein, 458 aa, Artificial
  Heavy chain
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 458 aa, Artificial
  Heavy chain
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 17 - protein, 458 aa, Artificial
  Heavy chain
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGDDSVFLFPPKPKDTLMISRTPEVTCVVVDVSDEDGEVKFNWYVDGVEVHNAKTKPREEQYNSTYRVVSVLTVLHQDWLNGKEYKCKVSNKALPRPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 458 aa, Artificial
  Heavy chain
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 458 aa, Artificial
  Heavy chain
QVQLQESGPGLVKPSQTLSLTCAISGDSVSSNNAVWNWIRQSPSRGLEWLGRTYYRSKWYNDYAESVKSRITVNPDTSKNQFSLHLKSVTPEDTAVFYCVRSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 20 - DNA, 1377 bp, Artificial
  Heavy chain
caggtacagctgcaggagtcgggtccaggactggtgaagccctcgcagaccctctcactcacctgtgccatctccggggacagtgtctctagcaacaatgctgtttggaactggatcaggcagtccccatcgagaggccttgagtggctgggaaggacatactacaggtccaagtggtataatgattatgcagaatctgtgaaaagtcgaataaccgtcaatccagacacatccaagaaccagttctccctgcacctgaagtctgtgactcccgaggacacggctgtgttttactgtgtacgatctggccacattacggtttttggagtgaatgttgacgcttttgatatgtggggccaagggacaatggtcaccgtctcttcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccactccccgaagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 21 - DNA, 1377 bp, Artificial
  Heavy chain
caggtacagctgcaggagtcgggtccaggactggtgaagccctcgcagaccctctcactcacctgtgccatctccggggacagtgtctctagcaacaatgctgtttggaactggatcaggcagtccccatcgagaggccttgagtggctgggaaggacatactacaggtccaagtggtataatgattatgcagaatctgtgaaaagtcgaataaccgtcaatccagacacatccaagaaccagttctccctgcacctgaagtctgtgactcccgaggacacggctgtgttttactgtgtacgatctggccacattacggtttttggagtgaatgttgacgcttttgatatgtggggccaagggacaatggtcaccgtctcttcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggtaaatga

SEQ ID NO: 22 - DNA, 1377 bp, Artificial
  Heavy chain
caggtacagctgcaggagtcgggtccaggactggtgaagccctcgcagaccctctcactcacctgtgccatctccggggacagtgtctctagcaacaatgctgtttggaactggatcaggcagtccccatcgagaggccttgagtggctgggaaggacatactacaggtccaagtggtataatgattatgcagaatctgtgaaaagtcgaataaccgtcaatccagacacatccaagaaccagttctccctgcacctgaagtctgtgactcccgaggacacggctgtgttttactgtgtacgatctggccacattacggtttttggagtgaatgttgacgcttttgatatgtggggccaagggacaatggtcaccgtctcttcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccactccccgaagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggtaaatga

SEQ ID NO: 23 - DNA, 1377 bp, Artificial
  Heavy chain
caggtacagctgcaggagtcgggtccaggactggtgaagccctcgcagaccctctcactcacctgtgccatctccggggacagtgtctctagcaacaatgctgtttggaactggatcaggcagtccccatcgagaggccttgagtggctgggaaggacatactacaggtccaagtggtataatgattatgcagaatctgtgaaaagtcgaataaccgtcaatccagacacatccaagaaccagttctccctgcacctgaagtctgtgactcccgaggacacggctgtgttttactgtgtacgatctggccacattacggtttttggagtgaatgttgacgcttttgatatgtggggccaagggacaatggtcaccgtctcttcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccactccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 24 - DNA, 1377 bp, Artificial
  Heavy chain
caggtacagctgcaggagtcgggtccaggactggtgaagccctcgcagaccctctcactcacctgtgccatctccggggacagtgtctctagcaacaatgctgtttggaactggatcaggcagtccccatcgagaggccttgagtggctgggaaggacatactacaggtccaagtggtataatgattatgcagaatctgtgaaaagtcgaataaccgtcaatccagacacatccaagaaccagttctccctgcacctgaagtctgtgactcccgaggacacggctgtgttttactgtgtacgatctggccacattacggtttttggagtgaatgttgacgcttttgatatgtggggccaagggacaatggtcaccgtctcttcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggcgggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 25 - DNA, 1377 bp, Artificial
  Heavy chain
caggtacagctgcaggagtcgggtccaggactggtgaagccctcgcagaccctctcactcacctgtgccatctccggggacagtgtctctagcaacaatgctgtttggaactggatcaggcagtccccatcgagaggccttgagtggctgggaaggacatactacaggtccaagtggtataatgattatgcagaatctgtgaaaagtcgaataaccgtcaatccagacacatccaagaaccagttctccctgcacctgaagtctgtgactcccgaggacacggctgtgttttactgtgtacgatctggccacattacggtttttggagtgaatgttgacgcttttgatatgtggggccaagggacaatggtcaccgtctcttcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggcgggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggtaaatga

SEQ ID NO: 26 - protein, 8 aa, Artificial
  CDRH1
GFTFSTYA

SEQ ID NO: 27 - protein, 8 aa, Artificial
  CDRH2
ISYDANYK

SEQ ID NO: 28 - protein, 22 aa, Artificial
  CDRH3
AKDSQLRSLLYFEWLSQGYFDY

SEQ ID NO: 29 - protein, 12 aa, Artificial
  CDRL1
SSQSVTFNYKNY

SEQ ID NO: 30 - protein, 3 aa, Artificial
  CDRL2
WAS

SEQ ID NO: 31 - protein, 9 aa, Artificial
  CDRL3
QQHYRTPPT

SEQ ID NO: 32 - protein, 129 aa, Artificial
  VH
QVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSR

SEQ ID NO: 33 - protein, 113 aa, Artificial
  VL
YGDIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVDSR

SEQ ID NO: 34 - protein, 478 aa, Artificial
  Heavy chain
MEFGLSWVFLVALLRGVQCQVQLVESGGGVVQPGRSLRLSCAASGFTFSTYAMHWVRQAPGKGLEWVAVISYDANYKYYADSVKGRFTISRDNSKNTLYLQMNSLRAEDTAVYYCAKDSQLRSLLYFEWLSQGYFDYWGQGTLVTVSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 238 aa, Artificial
  Light chain
MVLQTQVFISLLLWISGAYGDIVMTQSPDSLAVSLGERATINCKSSQSVTFNYKNYLAWYQQKPGQPPKLLIYWASTRESGVPDRFSGSGSGTDFTLTISSLQAEDVAVYYCQQHYRTPPTFGQGTKVDSRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 8 aa, Artificial
  CDRH1
GGSFGGYY

SEQ ID NO: 37 - protein, 7 aa, Artificial
  CDRH2
INHSGST

SEQ ID NO: 38 - protein, 18 aa, Artificial
  CDRH3
ARGRGGYATYYYYYYVDV

SEQ ID NO: 39 - protein, 6 aa, Artificial
  CDRL1
QSVSSY

SEQ ID NO: 40 - protein, 3 aa, Artificial
  CDRL2
DAS

SEQ ID NO: 41 - protein, 8 aa, Artificial
  CDRL3
QQRSNWLT

SEQ ID NO: 42 - protein, 124 aa, Artificial
  VH
QVQLQQWGAGLLKPSETLSLTCAVYGGSFGGYYWNWIRQPPGKGLEWIGEINHSGSTNYNPSLKSRVTLSVDTSKNQVSLNVSSVTAADTAVYYCARGRGGYATYYYYYYVDVWGKGTTVTVSS

SEQ ID NO: 43 - protein, 106 aa, Artificial
  VL
EIVLTQSPATLSLSPGERATLSCRASQSVSSYLAWYQQKPGQAPRLLIYDASKRATGIPARFSGSGSGTDFTLTISSLEPEDFAVYYCQQRSNWLTFGGGTKVELE

SEQ ID NO: 44 - protein, 472 aa, Artificial
  Heavy chain
MGWSCIILFLVATATGVHSQVQLQQWGAGLLKPSETLSLTCAVYGGSFGGYYWNWIRQPPGKGLEWIGEINHSGSTNYNPSLKSRVTLSVDTSKNQVSLNVSSVTAADTAVYYCARGRGGYATYYYYYYVDVWGKGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 231 aa, Artificial
  Light chain
MGWSCIILFLVATATGHSEIVLTQSPATLSLSPGERATLSCRASQSVSSYLAWYQQKPGQAPRLLIYDASKRATGIPARFSGSGSGTDFTLTISSLEPEDFAVYYCQQRSNWLTFGGGTKVELERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 8 aa, Artificial
  CDRH1
GSSISNYY

SEQ ID NO: 47 - protein, 7 aa, Artificial
  CDRH2
IYYGGNT

SEQ ID NO: 48 - protein, 14 aa, Artificial
  CDRH3
ARASCSGGYCILDY

SEQ ID NO: 49 - protein, 6 aa, Artificial
  CDRL1
QNIYKY

SEQ ID NO: 50 - protein, 3 aa, Artificial
  CDRL2
AAS

SEQ ID NO: 51 - protein, 9 aa, Artificial
  CDRL3
QQSYSPPLT

SEQ ID NO: 52 - protein, 120 aa, Artificial
  VH
QVQLQESGPGLVKPSETLSLTCTVSGSSISNYYWSWIRQSPGKGLEWIGFIYYGGNTKYNPSLKSRVTISQDTSKSQVSLTMSSVTAAESAVYFCARASCSGGYCILDYWGQGTLVTVSS

SEQ ID NO: 53 - protein, 107 aa, Artificial
  VL
DIQMTQSPSSLSASVGDRVTITCRASQNIYKYLNWYQQRPGKAPKGLISAASGLQSGVPSRFSGSGSGTDFTLTITSLQPEDFATYYCQQSYSPPLTFGGGTRVEIK

SEQ ID NO: 54 - protein, 469 aa, Artificial
  Heavy chain
MGWSCIILFLVATATGAHSQVQLQESGPGLVKPSETLSLTCTVSGSSISNYYWSWIRQSPGKGLEWIGFIYYGGNTKYNPSLKSRVTISQDTSKSQVSLTMSSVTAAESAVYFCARASCSGGYCILDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 232 aa, Artificial
  Light chain
MGWSCIILFLVATATGVHDIQMTQSPSSLSASVGDRVTITCRASQNIYKYLNWYQQRPGKAPKGLISAASGLQSGVPSRFSGSGSGTDFTLTITSLQPEDFATYYCQQSYSPPLTFGGGTRVEIKRTVDAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 8 aa, Artificial
  CDRH1
GYIFTDYA

SEQ ID NO: 57 - protein, 8 aa, Artificial
  CDRH2
ISTYTGKT

SEQ ID NO: 58 - protein, 12 aa, Artificial
  CDRH3
ARRGDYDAWFAY

SEQ ID NO: 59 - protein, 12 aa, Artificial
  CDRL1
QRLLYSSDQKNY

SEQ ID NO: 60 - protein, 3 aa, Artificial
  CDRL2
WAS

SEQ ID NO: 61 - protein, 9 aa, Artificial
  CDRL3
QQYYTYPLT

SEQ ID NO: 62 - protein, 119 aa, Artificial
  VH
QVQLQQSGPEVVRPGVSVKISCKGSGYIFTDYAMHWVKQSHAKSLEWIGVISTYTGKTNYSQKFKGKATMTVDKSSSTAYMELARLTSEDSSVYYCARRGDYDAWFAYWGQGTLVTVSS

SEQ ID NO: 63 - protein, 113 aa, Artificial
  VL
DIVMSQSPSSLAVSVGEKVSMTCKSSQRLLYSSDQKNYLAWYQQKPGQSPKVLIYWASTRVSGVPDRFTGSESGTDFTLTISSVKAEDLAVYYCQQYYTYPLTFGAGTKLELK

SEQ ID NO: 64 - protein, 468 aa, Artificial
  Heavy chain
MGWSCIILFLVATATGAHSQVQLQQSGPEVVRPGVSVKISCKGSGYIFTDYAMHWVKQSHAKSLEWIGVISTYTGKTNYSQKFKGKATMTVDKSSSTAYMELARLTSEDSSVYYCARRGDYDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 238 aa, Artificial
  Light chain
MGWSCIILFLVATATGVHDIVMSQSPSSLAVSVGEKVSMTCKSSQRLLYSSDQKNYLAWYQQKPGQSPKVLIYWASTRVSGVPDRFTGSESGTDFTLTISSVKAEDLAVYYCQQYYTYPLTFGAGTKLELKRTVDAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 377 aa, Artificial
  Heavy Chain
MNSLRAEDTAVYYCAKDSQLRSLLYFEWLSQGYFDYWGQGTLVTVSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 377 aa, Artificial
  Heavy Chain
MNSLRAEDTAVYYCAKDSQLRSLLYFEWLSQGYFDYWGQGTLVTVSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 377 aa, Artificial
  Heavy Chain
MNSLRAEDTAVYYCAKDSQLRSLLYFEWLSQGYFDYWGQGTLVTVSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 377 aa, Artificial
  Heavy Chain
MNSLRAEDTAVYYCAKDSQLRSLLYFEWLSQGYFDYWGQGTLVTVSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 377 aa, Artificial
  Heavy Chain
MNSLRAEDTAVYYCAKDSQLRSLLYFEWLSQGYFDYWGQGTLVTVSRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 472 aa, Artificial; identical to SEQ ID NO: 44
  Heavy Chain

SEQ ID NO: 72 - protein, 472 aa, Artificial
  Heavy Chain
MGWSCIILFLVATATGVHSQVQLQQWGAGLLKPSETLSLTCAVYGGSFGGYYWNWIRQPPGKGLEWIGEINHSGSTNYNPSLKSRVTLSVDTSKNQVSLNVSSVTAADTAVYYCARGRGGYATYYYYYYVDVWGKGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 472 aa, Artificial
  Heavy Chain
MGWSCIILFLVATATGVHSQVQLQQWGAGLLKPSETLSLTCAVYGGSFGGYYWNWIRQPPGKGLEWIGEINHSGSTNYNPSLKSRVTLSVDTSKNQVSLNVSSVTAADTAVYYCARGRGGYATYYYYYYVDVWGKGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 472 aa, Artificial
  Heavy Chain
MGWSCIILFLVATATGVHSQVQLQQWGAGLLKPSETLSLTCAVYGGSFGGYYWNWIRQPPGKGLEWIGEINHSGSTNYNPSLKSRVTLSVDTSKNQVSLNVSSVTAADTAVYYCARGRGGYATYYYYYYVDVWGKGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 472 aa, Artificial
  Heavy Chain
MGWSCIILFLVATATGVHSQVQLQQWGAGLLKPSETLSLTCAVYGGSFGGYYWNWIRQPPGKGLEWIGEINHSGSTNYNPSLKSRVTLSVDTSKNQVSLNVSSVTAADTAVYYCARGRGGYATYYYYYYVDVWGKGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 469 aa, Artificial; identical to SEQ ID NO: 54
  Heavy Chain

SEQ ID NO: 77 - protein, 469 aa, Artificial
  Heavy Chain
MGWSCIILFLVATATGAHSQVQLQESGPGLVKPSETLSLTCTVSGSSISNYYWSWIRQSPGKGLEWIGFIYYGGNTKYNPSLKSRVTISQDTSKSQVSLTMSSVTAAESAVYFCARASCSGGYCILDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 469 aa, Artificial
  Heavy Chain
MGWSCIILFLVATATGAHSQVQLQESGPGLVKPSETLSLTCTVSGSSISNYYWSWIRQSPGKGLEWIGFIYYGGNTKYNPSLKSRVTISQDTSKSQVSLTMSSVTAAESAVYFCARASCSGGYCILDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 469 aa, Artificial
  Heavy Chain
MGWSCIILFLVATATGAHSQVQLQESGPGLVKPSETLSLTCTVSGSSISNYYWSWIRQSPGKGLEWIGFIYYGGNTKYNPSLKSRVTISQDTSKSQVSLTMSSVTAAESAVYFCARASCSGGYCILDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 468 aa, Artificial; identical to SEQ ID NO: 64
  Heavy Chain

SEQ ID NO: 81 - protein, 468 aa, Artificial
  Heavy Chain
MGWSCIILFLVATATGAHSQVQLQQSGPEVVRPGVSVKISCKGSGYIFTDYAMHWVKQSHAKSLEWIGVISTYTGKTNYSQKFKGKATMTVDKSSSTAYMELARLTSEDSSVYYCARRGDYDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 468 aa, Artificial
  Heavy Chain
MGWSCIILFLVATATGAHSQVQLQQSGPEVVRPGVSVKISCKGSGYIFTDYAMHWVKQSHAKSLEWIGVISTYTGKTNYSQKFKGKATMTVDKSSSTAYMELARLTSEDSSVYYCARRGDYDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 468 aa, Artificial
  Heavy Chain
MGWSCIILFLVATATGAHSQVQLQQSGPEVVRPGVSVKISCKGSGYIFTDYAMHWVKQSHAKSLEWIGVISTYTGKTNYSQKFKGKATMTVDKSSSTAYMELARLTSEDSSVYYCARRGDYDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 468 aa, Artificial
  Heavy Chain
MGWSCIILFLVATATGAHSQVQLQQSGPEVVRPGVSVKISCKGSGYIFTDYAMHWVKQSHAKSLEWIGVISTYTGKTNYSQKFKGKATMTVDKSSSTAYMELARLTSEDSSVYYCARRGDYDAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 24 bp, Artificial
  CDRH1
ggattcaccttcagtagttcctgg

SEQ ID NO: 86 - DNA, 24 bp, Artificial
  CDRH2
attaatagtggtgggaatttcaaa

SEQ ID NO: 87 - DNA, 48 bp, Artificial
  CDRH3
gcaagagatcatgactacggtgactacagagggaacgcgtatgatatc

SEQ ID NO: 88 - DNA, 18 bp, Artificial
  CDRL1
caggacattagcaactat

SEQ ID NO: 89 - DNA, 9 bp, Artificial
  CDRL2
gatacatcc

SEQ ID NO: 90 - DNA, 15 bp, Artificial
  CDRL3
cagcagcttgatagt

SEQ ID NO: 91 - DNA, 296 bp, Artificial
  VH
gaggtgcagctggtggagtcggggggagacttagttcagccgggggggtccctgagactctcctgtgcaggctctggattcaccttcagtagttcctggatgcactgggtccgccaagctccagggaaggggctggtgtgggtctcacgtattaatagtggtgggaatttcaaaaaatacgcggactccgtgaggggccgattcaccatctccagagacaacaccaggaacaccctatatctgcatatgagcagtctgagacacgaggacacggctctttattactgtgcaagaga

SEQ ID NO: 92 - DNA, 272 bp, Artificial
  VL
gacatccagatgacccagtctccatcctccctgtctgcatctgtgggagacagagtcaccatcacttgccaggcgagtcaggacattagcaactatttcaattggtatcagcagaaaccagggaaagcccctaagctcctaatcttcgatacatccaagttggaaacaggggtcccatcaaggttcagtggaagacaatctgggacagattatactttcaccatcagcagcctgcagcctgaagatattgcaacatatttctgtcagcagct

SEQ ID NO: 93 - DNA, 1416 bp, Artificial
  Heavy chain
atgggatggtcatgtatcatcctttttctagtagcaactgcaaccggtgtacattctgaggtgcagctggtggagtcggggggagacttagttcagccgggggggtccctgagactctcctgtgcaggctctggattcaccttcagtagttcctggatgcactgggtccgccaagctccagggaaggggctggtgtgggtctcacgtattaatagtggtgggaatttcaaaaaatacgcggactccgtgaggggccgattcaccatctccagagacaacaccaggaacaccctatatctgcatatgagcagtctgagacacgaggacacggctctttattactgtgcaagagatcatgactacggtgactacagagggaacgcgtatgatatctggggccaagggacaatggtcaccgtctcgagctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 94 - DNA, 624 bp, Artificial
  Light chain
atgacccagtctccatcctccctgtctgcatctgtgggagacagagtcaccatcacttgccaggcgagtcaggacattagcaactatttcaattggtatcagcagaaaccagggaaagcccctaagctcctaatcttcgatacatccaagttggaaacaggggtcccatcaaggttcagtggaagacaatctgggacagattatactttcaccatcagcagcctgcagcctgaagatattgcaacatatttctgtcagcagcttgatagtttcggcggagggaccaaggtggagctcgagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 95 - protein, 8 aa, Artificial
  CDRH1
GFTFSSSW

SEQ ID NO: 96 - protein, 8 aa, Artificial
  CDRH2
INSGGNFK

SEQ ID NO: 97 - protein, 16 aa, Artificial
  CDRH3
ARDHDYGDYRGNAYDI

SEQ ID NO: 98 - protein, 6 aa, Artificial
  CDRL1
QDISNY

SEQ ID NO: 99 - protein, 3 aa, Artificial
  CDRL2
DTS

SEQ ID NO: 100 - protein, 5 aa, Artificial
  VH
QQLDS

SEQ ID NO: 101 - protein, 98 aa, Artificial
  VH
EVQLVESGGDLVQPGGSLRLSCAGSGFTFSSSWMHWVRQAPGKGLVWVSRINSGGNFKKYADSVRGRFTISRDNTRNTLYLHMSSLRHEDTALYYCAR

SEQ ID NO: 102 - protein, 90 aa, Artificial
  VL
DIQMTQSPSSLSASVGDRVTITCQASQDISNYFNWYQQKPGKAPKLLIFDTSKLETGVPSRFSGRQSGTDYTFTISSLQPEDIATYFCQQ

SEQ ID NO: 103 - protein, 471 aa, Artificial
  Heavy chain
MGWSCIILFLVATATGVHSEVQLVESGGDLVQPGGSLRLSCAGSGFTFSSSWMHWVRQAPGKGLVWVSRINSGGNFKKYADSVRGRFTISRDNTRNTLYLHMSSLRHEDTALYYCARDHDYGDYRGNAYDIWGQGTMVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 207 aa, Artificial
  Light chain
MTQSPSSLSASVGDRVTITCQASQDISNYFNWYQQKPGKAPKLLIFDTSKLETGVPSRFSGRQSGTDYTFTISSLQPEDIATYFCQQLDSFGGGTKVELERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - DNA, 30 bp, Artificial
  CDRH1
ggtggctccatcagaagtggtattcactac

SEQ ID NO: 106 - DNA, 21 bp, Artificial
  CDRH2
atccactacagtgagaatacc

SEQ ID NO: 107 - DNA, 75 bp, Artificial
  CDRH3
gcgagagcggcaaaagagtctctttgtattggtggtagctgcgactcaaactacgaacactacggtttggacgtc

SEQ ID NO: 108 - DNA, 18 bp, Artificial
  CDRL1
cagagcataaggaactat

SEQ ID NO: 109 - DNA, 9 bp, Artificial
  CDRL2
gctgtatcc

SEQ ID NO: 110 - DNA, 30 bp, Artificial
  CDRL3
caacagagttacagcaccctcccgtacact

SEQ ID NO: 111 - DNA, 298 bp, Artificial
  VH
caggtgcagctgcaggagtcgggcccaggactggtgaagccttcacagaccctgtccctcacctgcactgtctctggtggctccatcagaagtggtattcactactggagctggatccgccaattcccagggaagggcctggagtggattggactcatccactacagtgagaatacccaccacaacccgtccctcaagagtcgagttgccatgtcagtagacacgtctaagaaccagttctccctgaccctgagctctgtgacggccgcggacacggccgtctattattgtgcgagag

SEQ ID NO: 112 - DNA, 287 bp, Artificial
  VL
gacatccagatgacccagtctccatcctccctgtctgcatctataggagacagagtcaccatcacttgccgggcaagtcagagcataaggaactatttaaattggtatcagcagaaaccaggcaaagcccctaaactcgtgatctatgctgtatccaatttgcaaagtggggtcccatcaaggttcagtggcagtggatctgggacagatttcacgctcaccatcagcagtctgcaacctgaagatcttgcaacctactactgtcaacagagttacagcaccctccc

SEQ ID NO: 113 - DNA, 1446 bp, Artificial
  Heavy chain
atgggatggtcatgtatcatcctttttctagtagcaactgcaaccggtgtacattcccaggtgcagctgcaggagtcgggcccaggactggtgaagccttcacagaccctgtccctcacctgcactgtctctggtggctccatcagaagtggtattcactactggagctggatccgccaattcccagggaagggcctggagtggattggactcatccactacagtgagaatacccaccacaacccgtccctcaagagtcgagttgccatgtcagtagacacgtctaagaaccagttctccctgaccctgagctctgtgacggccgcggacacggccgtctattattgtgcgagagcggcaaaagagtctctttgtattggtggtagctgcgactcaaactacgaacactacggtttggacgtctggggccaagggaccacggtcaccgtctcgagctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 114 - DNA, 711 bp, Artificial
  Light chain
atgggatggtcatgtatcatcctttttctagtagcaactgcaaccggtgtacattctgacatccagatgacccagtctccatcctccctgtctgcatctataggagacagagtcaccatcacttgccgggcaagtcagagcataaggaactatttaaattggtatcagcagaaaccaggcaaagcccctaaactcgtgatctatgctgtatccaatttgcaaagtggggtcccatcaaggttcagtggcagtggatctgggacagatttcacgctcaccatcagcagtctgcaacctgaagatcttgcaacctactactgtcaacagagttacagcaccctcccgtacacttttggccaggggaccaaggtggagatcaaactcgagcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 115 - protein, 10 aa, Artificial
  CDRH1
GGSIRSGIHY

SEQ ID NO: 116 - protein, 7 aa, Artificial
  CDRH2
IHYSENT

SEQ ID NO: 117 - protein, 25 aa, Artificial
  CDRH3
ARAAKESLCIGGSCDSNYEHYGLDV

SEQ ID NO: 118 - protein, 6 aa, Artificial
  CDRL1
QSIRNY

SEQ ID NO: 119 - protein, 3 aa, Artificial
  CDRL2
AVS

SEQ ID NO: 120 - protein, 10 aa, Artificial
  CDRL3
QQSYSTLPYT

SEQ ID NO: 121 - protein, 99 aa, Artificial
  VH
QVQLQESGPGLVKPSQTLSLTCTVSGGSIRSGIHYWSWIRQFPGKGLEWIGLIHYSENTHHNPSLKSRVAMSVDTSKNQFSLTLSSVTAADTAVYYCAR

SEQ ID NO: 122 - protein, 95 aa, Artificial
  VL
DIQMTQSPSSLSASIGDRVTITCRASQSIRNYLNWYQQKPGKAPKLVIYAVSNLQSGVPSRFSGSGSGTDFTLTISSLQPEDLATYYCQQSYSTL

SEQ ID NO: 123 - protein, 481 aa, Artificial
  Heavy chain
MGWSCIILFLVATATGVHSQVQLQESGPGLVKPSQTLSLTCTVSGGSIRSGIHYWSWIRQFPGKGLEWIGLIHYSENTHHNPSLKSRVAMSVDTSKNQFSLTLSSVTAADTAVYYCARAAKESLCIGGSCDSNYEHYGLDVWGQGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 236 aa, Artificial
  Light chain
MGWSCIILFLVATATGVHSDIQMTQSPSSLSASIGDRVTITCRASQSIRNYLNWYQQKPGKAPKLVIYAVSNLQSGVPSRFSGSGSGTDFTLTISSLQPEDLATYYCQQSYSTLPYTFGQGTKVEIKLERADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 125 - protein, 471 aa, Artificial
  Heavy Chain
MGWSCIILFLVATATGVHSEVQLVESGGDLVQPGGSLRLSCAGSGFTFSSSWMHWVRQAPGKGLVWVSRINSGGNFKKYADSVRGRFTISRDNTRNTLYLHMSSLRHEDTALYYCARDHDYGDYRGNAYDIWGQGTMVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 471 aa, Artificial
  Heavy Chain
MGWSCIILFLVATATGVHSEVQLVESGGDLVQPGGSLRLSCAGSGFTFSSSWMHWVRQAPGKGLVWVSRINSGGNFKKYADSVRGRFTISRDNTRNTLYLHMSSLRHEDTALYYCARDHDYGDYRGNAYDIWGQGTMVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 471 aa, Artificial
  Heavy Chain
MGWSCIILFLVATATGVHSEVQLVESGGDLVQPGGSLRLSCAGSGFTFSSSWMHWVRQAPGKGLVWVSRINSGGNFKKYADSVRGRFTISRDNTRNTLYLHMSSLRHEDTALYYCARDHDYGDYRGNAYDIWGQGTMVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 471 aa, Artificial
  Heavy Chain
MGWSCIILFLVATATGVHSEVQLVESGGDLVQPGGSLRLSCAGSGFTFSSSWMHWVRQAPGKGLVWVSRINSGGNFKKYADSVRGRFTISRDNTRNTLYLHMSSLRHEDTALYYCARDHDYGDYRGNAYDIWGQGTMVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 471 aa, Artificial
  Heavy Chain
MGWSCIILFLVATATGVHSEVQLVESGGDLVQPGGSLRLSCAGSGFTFSSSWMHWVRQAPGKGLVWVSRINSGGNFKKYADSVRGRFTISRDNTRNTLYLHMSSLRHEDTALYYCARDHDYGDYRGNAYDIWGQGTMVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 481 aa, Artificial
  Heavy Chain
MGWSCIILFLVATATGVHSQVQLQESGPGLVKPSQTLSLTCTVSGGSIRSGIHYWSWIRQFPGKGLEWIGLIHYSENTHHNPSLKSRVAMSVDTSKNQFSLTLSSVTAADTAVYYCARAAKESLCIGGSCDSNYEHYGLDVWGQGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 481 aa, Artificial
  Heavy Chain
MGWSCIILFLVATATGVHSQVQLQESGPGLVKPSQTLSLTCTVSGGSIRSGIHYWSWIRQFPGKGLEWIGLIHYSENTHHNPSLKSRVAMSVDTSKNQFSLTLSSVTAADTAVYYCARAAKESLCIGGSCDSNYEHYGLDVWGQGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 481 aa, Artificial
  Heavy Chain
MGWSCIILFLVATATGVHSQVQLQESGPGLVKPSQTLSLTCTVSGGSIRSGIHYWSWIRQFPGKGLEWIGLIHYSENTHHNPSLKSRVAMSVDTSKNQFSLTLSSVTAADTAVYYCARAAKESLCIGGSCDSNYEHYGLDVWGQGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 481 aa, Artificial
  Heavy Chain
MGWSCIILFLVATATGVHSQVQLQESGPGLVKPSQTLSLTCTVSGGSIRSGIHYWSWIRQFPGKGLEWIGLIHYSENTHHNPSLKSRVAMSVDTSKNQFSLTLSSVTAADTAVYYCARAAKESLCIGGSCDSNYEHYGLDVWGQGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 481 aa, Artificial
  Heavy Chain
MGWSCIILFLVATATGVHSQVQLQESGPGLVKPSQTLSLTCTVSGGSIRSGIHYWSWIRQFPGKGLEWIGLIHYSENTHHNPSLKSRVAMSVDTSKNQFSLTLSSVTAADTAVYYCARAAKESLCIGGSCDSNYEHYGLDVWGQGTTVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0298230 A1
(43) Pub. Date: Sep. 22, 2022

(54) ANTIBODIES AND METHODS FOR TREATMENT OF VIRAL INFECTIONS

(71) Applicant: The Rockefeller University, New York, NY (US)

(72) Inventors: Jeffrey V. Ravetch, New York, NY (US); Stylianos Bournazos, New York, NY (US)

(73) Assignee: The Rockefeller University, New York, NY (US)

(21) Appl. No.: 17/617,901

(22) PCT Filed: Jun. 4, 2020
(86) PCT No.: PCT/US20/36183

§ 371 Date: Dec. 9, 2021

Related U.S. Application Data
(60) Provisional application No. 62/859,795, filed on Jun. 11, 2019.

(51) Int. Cl. C07K 16/10; A61K 31/7088

(52) U.S. Cl. CPC C07K 16/1018; A61K 31/7088; A61K 2039/505

(57) ABSTRACT

The present invention provides antibodies that are capable of activating dendritic cell maturation and/or inducing a protective CDS response. The disclosed antibodies can be used to treat or inhibit viral infections, including prophylaxis and treatment of influenza A infection. The invention also provides nucleic acids that encode and immortalized B cells and cultured plasma cells that produce such antibodies.

### CROSS-REFERENCE TO RELATED APPLICATIONS

This application claims priority under 35 U.S.C. § 119(e) to U.S. Provisional Patent Application No. 62/859,795, filed Jun. 11, 2019. The foregoing application is incorporated by reference herein is its entirety.

### STATEMENT REGARDING FEDERALLY SPONSORED RESEARCH

This invention was made with government support under AI129795 awarded by National Institutes of Health. The government has certain rights in the invention.

### FIELD OF THE INVENTION

The invention relates to antibodies capable of activating dendritic cell maturation and/or inducing a protective CD8 response and to the use of such antibodies. In particular, the invention relates to the prophylaxis and treatment of viral infections, such as influenza A infection.

### BACKGROUND OF THE INVENTION

Influenza is an infectious disease, which spreads around the world in yearly outbreaks resulting per year in about three to five million cases of severe illness and about 290,000 to 650,000 respiratory deaths (WHO, Influenza (Seasonal) Fact sheet, Nov. 6, 2018). The most common symptoms include: a sudden onset of fever, cough (usually dry), headache, muscle and joint pain, severe malaise (feeling unwell), sore throat, and a runny nose. The incubation period varies between one to four days, although usually the symptoms begin about two days after exposure to the virus. Complications of influenza may include pneumonia, sinus infections, and worsening of previous health problems such as asthma or heart failure, sepsis or exacerbation of chronic underlying diseases.

Influenza is caused by influenza virus, an antigenically and genetically diverse group of viruses of the family Orthomyxoviridae that contains a negative-sense, single-stranded, segmented RNA genome. Of the four types of influenza virus (A, B, C, and D), three types (A, B, and C) affect humans. Influenza type A viruses are the most virulent human pathogens and cause the severest disease. Influenza A viruses can be categorized based on the different subtypes of major surface proteins present: Hemagglutinin (HA) and Neuraminidase (NA). There are at least 18 influenza A subtypes defined by their hemagglutinin (“HA”) proteins. The HAs can be classified into two groups. Group 1 contains H1, H2, H5, H6, H8, H9, H11, H12, H13, H16, and H17 subtypes, and group 2 includes H3, H4, H7, H10, H14, and H15 subtypes. While all subtypes are present in birds, mostly H1, H2, and H3 subtypes cause disease in humans. H5, H7, and H9 subtypes are causing sporadic severe infections in humans and may generate a new pandemic. Influenza A viruses continuously evolve, generating new variants, a phenomenon called antigenic drift. As a consequence, antibodies produced in response to past viruses are poorly- or non-protective against new drifted viruses. A consequence is that a new vaccine has to be produced every year against H1 and H3 viruses that are predicted to emerge, a process that is very costly as well as not always efficient. The same applies to the production of an H5 influenza vaccine.

HA is a major surface protein of influenza A virus, which is the main target of neutralizing antibodies that are induced by infection or vaccination. HA is responsible for binding the virus to cells with sialic acid on the membranes, such as cells in the upper respiratory tract or erythrocytes. In addition, HA mediates the fusion of the viral envelope with the endosome membrane, after the pH has been reduced. HA is a homotrimeric integral membrane glycoprotein. The HA trimer is composed of three identical monomers, each made of an intact HA0 single polypeptide chain with HA1 and HA2 regions linked by 2 disulfide bridges. Each HA2 region adopts alpha-helical coiled-coil structure and primarily forms the “stem” or “stalk” region of HA, while the HA1 region is a small globular domain containing a mix of α/β structures (“head” region of HA). The globular HA head region mediates binding to the sialic acid receptor, while the HA stem mediates the subsequent fusion between the viral and cellular membranes that is triggered in endosomes by the low pH. While the immunodominant HA globular head domain has high plasticity with distinct antigenic sites undergoing constant antigenic drift, the HA stem region is relatively conserved among subtypes. Current influenza vaccines mostly induce an immune response against the immunodominant and variable HA head region, which evolves faster than the stem region of HA (Kirkpatrick E, et al. Sci Rep. 2018 Jul. 11; 8(1):10432). Therefore, a particular influenza vaccine usually confers protection for no more than a few years, and annual re-development of influenza vaccines is required.

To overcome these problems, recently, a new class of influenza-neutralizing antibodies that target conserved sites in the HA stem were developed as influenza virus therapeutics. These antibodies targeting the stem region of HA are usually broader neutralizing compared to antibodies targeting the head region of HA. An overview of broadly neutralizing influenza A antibodies is provided in Corti D. and Lanzavecchia A., Annu. Rev. Immunol. 2013; 31:705-742. Okuno et al. immunized mice with influenza virus A/Okuda/57 (H2N2) and isolated a monoclonal antibody (C179) that binds to a conserved conformational epitope in HA2 and neutralizes the Group 1 H2, H1, and H5 subtype influenza A viruses in vitro and in vivo in animal models (Okuno et al., 1993; Smirnov et al., 1999; Smirnov et al., 2000). Further examples of HA-stem region targeting antibodies include CR6261 (Throsby M, et al. (2008). PLoS ONE 3(12); Friesen R U E, et al. (2010). PLoS ONE 5(2)), F10 (Sui J, et al. (March 2009). Nature Structural & Molecular Biology. 16 (3): 265-73), CR8020 (Ekiert D C, et al. Science 333(6044):843-50), FI6 (Corti D, et al. 2011. Science 333(6044):850-56), and CR9114 (Dreyfus C, et al. 2012. Science 337(6100):1343-48).

However, antibodies capable of reacting with the HA stem region of both group 1 and 2 subtypes are extremely rare and usually do not show complete coverage of all subtypes. Recently, antibody FY1 was described, which potently neutralizes group 1 and 2 influenza A viruses with unprecedented breadth (Kallewaard N L, et al. Cell. 2016; 166(3):596-608).

Thus, there remains a strong need for a novel antibody for treating or inhibiting viral infections, including influenza A infection.

### SUMMARY OF THE INVENTION

This disclosure addresses the need mentioned above in a number of aspects. In one aspect, this disclosure provides an isolated Fc receptor-dependent antibody or antigen binding portion thereof capable of activating dendritic cell maturation. In another aspect, this disclosure provides an isolated Fc receptor-dependent antibody or antigen binding portion thereof capable of inducing a protective CD8 response.

In some embodiments, the antibody or antigen binding portion thereof binds specifically to a viral antigen. In some embodiments, the viral antigen comprises an influenza virus antigen comprising hemagglutinin (HA) or neuraminidase (NA).

In some embodiments, the antibody or antigen binding portion thereof comprises (i) a heavy chain having a G236A mutation in a constant region thereof and (ii) an Fc region, wherein the Fc region activates FcγRIIa.

In some embodiments, the antibody or antigen binding portion thereof, comprises: (i) the heavy chain CDR1, CDR2, and CDR3 sequences as set forth in SEQ ID NO: 1, SEQ ID NO: 2, and SEQ ID NO: 3, respectively; and the light chain CDR1, CDR2, and CDR3 sequences as set forth in: SEQ ID NO: 4, SEQ ID NO: 5, and SEQ ID NO: 6, respectively; (ii) the heavy chain CDR1, CDR2, and CDR3 sequences as set forth in SEQ ID NO: 26, SEQ ID NO: 27, and SEQ ID NO: 28, respectively; and the light chain CDR1, CDR2, and CDR3 sequences as set forth in: SEQ ID NO: 29, SEQ ID NO: 30, and SEQ ID NO: 31, respectively; (iii) the heavy chain CDR1, CDR2, and CDR3 sequences as set forth in SEQ ID NO: 36, SEQ ID NO: 37, and SEQ ID NO: 38, respectively; and the light chain CDR1, CDR2, and CDR3 sequences as set forth in: SEQ ID NO: 39, SEQ ID NO: 40, and SEQ ID NO: 41, respectively; (iv) the heavy chain CDR1, CDR2, and CDR3 sequences as set forth in SEQ ID NO: 46, SEQ ID NO: 47, and SEQ ID NO: 48, respectively; and the light chain CDR1, CDR2, and CDR3 sequences as set forth in: SEQ ID NO: 49, SEQ ID NO: 50, and SEQ ID NO: 51, respectively; or (v) the heavy chain CDR1, CDR2, and CDR3 sequences as set forth in SEQ ID NO: 56, SEQ ID NO: 57, and SEQ ID NO: 58, respectively; and the light chain CDR1, CDR2, and CDR3 sequences as set forth in: SEQ ID NO: 59, SEQ ID NO: 60, and SEQ ID NO: 61, respectively, and the mutation G236A in the constant region of the heavy chain.

The antibody or antigen binding portion thereof may further include the mutations A330L and I332E in the constant region of the heavy chain. In some embodiments, the antibody or antigen binding portion thereof does not comprise the mutation S239D in the constant region of the heavy chain.

In some embodiments, the antibody or antigen binding portion thereof comprises a half-life increasing mutation in the constant region of the heavy chain, for example, the mutations M428L and N434S in the constant region of the heavy chain.

In another aspect, this disclosure also provides an antibody or antigen binding portion thereof comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations M428L and N434S in the constant region of the heavy chain.

The antibody or antigen binding portion thereof binds to hemagglutinin of an influenza A virus and thereby neutralizes infection with an influenza A virus.

In some embodiments, the antibody or antigen binding portion thereof can be afucosylated.

In some embodiments, the antibody or antigen binding portion thereof does not comprise the mutations G236R and L328R in the constant regions of the heavy chain. In some embodiments, the antibody or antigen binding portion thereof does not comprise the mutations G237D, P238D, H268D, P271G, and A330R in the constant regions of the heavy chain.

In some embodiments, the antibody or antigen binding portion thereof is a human antibody. In some embodiments, the antibody or antigen binding portion thereof is a monoclonal antibody, e.g., the IgG type. In some embodiments, the light chain of the antibody or antigen binding portion thereof is a kappa light chain.

In some embodiments, The antibody or antigen binding portion thereof of any one of the preceding claims, wherein the antibody or antigen binding portion thereof comprises: (i) a heavy chain variable region comprising an amino acid sequence having at least 75% identity (e.g., at least 75% identity, at least 80% identity, at least 85% identity, at least 90% identity, at least 95% identity) to SEQ ID NO: 7 and a light chain variable region comprising the amino acid sequence having at least 75% identity (e.g., at least 75% identity, at least 80% identity, at least 85% identity, at least 90% identity, at least 95% identity) to SEQ ID NO: 8; (ii) a heavy chain variable region comprising an amino acid sequence having at least 75% identity (e.g., at least 75% identity, at least 80% identity, at least 85% identity, at least 90% identity, at least 95% identity) to SEQ ID NO: 32 and a light chain variable region comprising the amino acid sequence having at least 75% identity (e.g., at least 75% identity, at least 80% identity, at least 85% identity, at least 90% identity, at least 95% identity) to SEQ ID NO: 33; (iii) a heavy chain variable region comprising an amino acid sequence having at least 75% identity (e.g., at least 75% identity, at least 80% identity, at least 85% identity, at least 90% identity, at least 95% identity) to SEQ ID NO: 42 and a light chain variable region comprising the amino acid sequence having at least 75% identity (e.g., at least 75% identity, at least 80% identity, at least 85% identity, at least 90% identity, at least 95% identity) to SEQ ID NO: 43; (iv) a heavy chain variable region comprising an amino acid sequence having at least 75% identity to SEQ ID NO: 52 and a light chain variable region comprising the amino acid sequence having at least 75% identity (e.g., at least 75% identity, at least 80% identity, at least 85% identity, at least 90% identity, at least 95% identity) to SEQ ID NO: 53; or (v) a heavy chain variable region comprising an amino acid sequence having at least 75% identity (e.g., at least 75% identity, at least 80% identity, at least 85% identity, at least 90% identity, at least 95% identity) to SEQ ID NO: 62 and a light chain variable region comprising the amino acid sequence having at least 75% identity (e.g., at least 75% identity, at least 80% identity, at least 85% identity, at least 90% identity, at least 95% identity) to SEQ ID NO: 63.

In some embodiments, the antibody or antigen binding portion thereof comprises: (i) the heavy chain CDR1, CDR2, and CDR3 sequences as set forth in SEQ ID NO: 1, SEQ ID NO: 2, and SEQ ID NO: 3, respectively, and the light chain CDR1, CDR2, and CDR3 sequences as set forth in: SEQ ID NO: 4, SEQ ID NO: 5, and SEQ ID NO: 6, respectively; and a heavy chain variable region comprising an amino acid sequence having at least 75% identity (e.g., at least 75% identity, at least 80% identity, at least 85% identity, at least 90% identity, at least 95% identity) to SEQ ID NO: 7 and a light chain variable region comprising the amino acid sequence having at least 75% identity (e.g., at least 75% identity, at least 80% identity, at least 85% identity, at least 90% identity, at least 95% identity) to SEQ ID NO: 8; (ii) the heavy chain CDR1, CDR2, and CDR3 sequences as set forth in SEQ ID NO: 26, SEQ ID NO: 27, and SEQ ID NO: 28, respectively, and the light chain CDR1, CDR2, and CDR3 sequences as set forth in: SEQ ID NO: 29, SEQ ID NO: 30, and SEQ ID NO: 31, respectively; and a heavy chain variable region comprising an amino acid sequence having at least 75% identity (e.g., at least 75% identity, at least 80% identity, at least 85% identity, at least 90% identity, at least 95% identity) to SEQ ID NO: 32 and a light chain variable region comprising the amino acid sequence having at least 75% identity (e.g., at least 75% identity, at least 80% identity, at least 85% identity, at least 90% identity, at least 95% identity) to SEQ ID NO: 33; (iii) the heavy chain CDR1, CDR2, and CDR3 sequences as set forth in SEQ ID NO: 36, SEQ ID NO: 37, and SEQ ID NO: 38, respectively, and the light chain CDR1, CDR2, and CDR3 sequences as set forth in: SEQ ID NO: 39, SEQ ID NO: 40, and SEQ ID NO: 41, respectively; and a heavy chain variable region comprising an amino acid sequence having at least 75% identity (e.g., at least 75% identity, at least 80% identity, at least 85% identity, at least 90% identity, at least 95% identity) to SEQ ID NO: 42 and a light chain variable region comprising the amino acid sequence having at least 75% identity (e.g., at least 75% identity, at least 80% identity, at least 85% identity, at least 90% identity, at least 95% identity) to SEQ ID NO: 43; (iv) the heavy chain CDR1, CDR2, and CDR3 sequences as set forth in SEQ ID NO: 46, SEQ ID NO: 47, and SEQ ID NO: 48, respectively, and the light chain CDR1, CDR2, and CDR3 sequences as set forth in: SEQ ID NO: 49, SEQ ID NO: 50, and SEQ ID NO: 51, respectively; and a heavy chain variable region comprising an amino acid sequence having at least 75% identity (e.g., at least 75% identity, at least 80% identity, at least 85% identity, at least 90% identity, at least 95% identity) to SEQ ID NO: 52 and a light chain variable region comprising the amino acid sequence having at least 75% identity (e.g., at least 75% identity, at least 80% identity, at least 85% identity, at least 90% identity, at least 95% identity) to SEQ ID NO: 53; or (v) the heavy chain CDR1, CDR2, and CDR3 sequences as set forth in SEQ ID NO: 56, SEQ ID NO: 57, and SEQ ID NO: 58, respectively, and the light chain CDR1, CDR2, and CDR3 sequences as set forth in: SEQ ID NO: 59, SEQ ID NO: 60, and SEQ ID NO: 61, respectively; and a heavy chain variable region comprising an amino acid sequence having at least 75% identity (e.g., at least 75% identity, at least 80% identity, at least 85% identity, at least 90% identity, at least 95% identity) to SEQ ID NO: 62 and a light chain variable region comprising the amino acid sequence having at least 75% identity (e.g., at least 75% identity, at least 80% identity, at least 85% identity, at least 90% identity, at least 95% identity) to SEQ ID NO: 63, and wherein the CDR sequences as defined are maintained.

In some embodiments, the antibody or antigen binding portion thereof comprises: (i) the heavy chain CDR1, CDR2, and CDR3 sequences as set forth in SEQ ID NO: 1, SEQ ID NO: 2, and SEQ ID NO: 3, respectively, and the light chain CDR1, CDR2, and CDR3 sequences as set forth in: SEQ ID NO: 4, SEQ ID NO: 5, and SEQ ID NO: 6, respectively; and a heavy chain variable region comprising an amino acid sequence as set forth in SEQ ID NO: 7 and a light chain variable region comprising the amino acid sequence as set forth in SEQ ID NO: 8; (ii) the heavy chain CDR1, CDR2, and CDR3 sequences as set forth in SEQ ID NO: 26, SEQ ID NO: 27, and SEQ ID NO: 28, respectively, and the light chain CDR1, CDR2, and CDR3 sequences as set forth in: SEQ ID NO: 29, SEQ ID NO: 30, and SEQ ID NO: 31, respectively; and a heavy chain variable region comprising an amino acid sequence set forth in SEQ ID NO: 32 and a light chain variable region comprising the amino acid sequence set forth in SEQ ID NO: 33; (iii) the heavy chain CDR1, CDR2, and CDR3 sequences as set forth in SEQ ID NO: 36, SEQ ID NO: 37, and SEQ ID NO: 38, respectively, and the light chain CDR1, CDR2, and CDR3 sequences as set forth in: SEQ ID NO: 39, SEQ ID NO: 40, and SEQ ID NO: 41, respectively; and a heavy chain variable region comprising an amino acid sequence set forth in SEQ ID NO: 42 and a light chain variable region comprising the amino acid sequence set forth in SEQ ID NO: 43; (iv) the heavy chain CDR1, CDR2, and CDR3 sequences as set forth in SEQ ID NO: 46, SEQ ID NO: 47, and SEQ ID NO: 48, respectively, and the light chain CDR1, CDR2, and CDR3 sequences as set forth in: SEQ ID NO: 49, SEQ ID NO: 50, and SEQ ID NO: 51, respectively; and a heavy chain variable region comprising an amino acid sequence set forth in SEQ ID NO: 52 and a light chain variable region comprising the amino acid sequence set forth in SEQ ID NO: 53; or (v) the heavy chain CDR1, CDR2, and CDR3 sequences as set forth in SEQ ID NO: 56, SEQ ID NO: 57, and SEQ ID NO: 58, respectively, and the light chain CDR1, CDR2, and CDR3 sequences as set forth in: SEQ ID NO: 59, SEQ ID NO: 60, and SEQ ID NO: 61, respectively; and a heavy chain variable region comprising an amino acid sequence set forth in SEQ ID NO: 62 and a light chain variable region comprising the amino acid sequence set forth in SEQ ID NO: 63.

In some embodiments, the CH2 region of the antibody or antigen binding portion thereof, as described above, does not comprise any further mutation in addition to G236A. In some embodiments, the CH2 region of the antibody or antigen binding portion thereof, as described above, does not comprise any further mutation in addition to G236A, A330L, and I332E. In some embodiments, the CH3 region of the antibody or antigen binding portion thereof, as described above, does not comprise any further mutation in addition to M428L and N434S. In some embodiments, the Fc region of the antibody or antigen binding portion thereof, as described above, does not comprise any further mutation in addition to G236A, A330L, and I332E and, optionally, M428L and N434S. In some embodiments, the Fc region of the antibody or antigen binding portion thereof, as described above, does not comprise any further mutation in addition to M428L and N434S.

In some embodiments, the antibody or antigen binding portion thereof comprises a light chain comprising an amino acid sequence as set forth in SEQ ID NO: 10 and a heavy chain comprising an amino acid sequence as set forth in SEQ ID NOs: 9, 13, 14, 18, or 19. In some embodiments, the antibody or antigen binding portion thereof comprises a light chain comprising an amino acid sequence as set forth in SEQ ID NO: 35 and a heavy chain comprising an amino acid sequence as set forth in SEQ ID NOs: 66, 68, 69 or 70. In some embodiments, the antibody or antigen binding portion thereof comprises a light chain comprising an amino acid sequence as set forth in SEQ ID NO: 45 and a heavy chain comprising an amino acid sequence as set forth in SEQ ID NOs: 73, 74 or 75. In some embodiments, the antibody or antigen binding portion thereof comprises a light chain comprising an amino acid sequence as set forth in SEQ ID NO: 55 and a heavy chain comprising an amino acid sequence as set forth in SEQ ID NOs: 77, 78 or 79. In some embodiments, the antibody or antigen binding portion thereof comprises a light chain comprising an amino acid sequence as set forth in SEQ ID NO: 65 and a heavy chain comprising an amino acid sequence as set forth in SEQ ID NOs: 81, 82, 83 or 84.

In another aspect, this disclosure provides the antibody or antigen binding portion thereof, as described above, for use in prophylaxis or treatment of infection with influenza A virus.

In some embodiments, the antibody or antigen binding portion thereof is administered prophylactically or therapeutically.

Also within the scope of this disclosure are: a nucleic acid molecule comprising a polynucleotide encoding the antibody or antigen binding portion thereof as described above; a vector comprising the nucleic acid molecule as described; and a cell expressing the disclosed antibody or antigen binding portion thereof or comprising the vector as described.

In another aspect, this disclosure also provides a pharmaceutical composition comprising the antibody or antigen binding portion thereof, the nucleic acid, the vector, or the cell, as described above, and, optionally, a pharmaceutically acceptable diluent or carrier.

Also provided is the use of the antibody or antigen binding portion thereof, the nucleic acid, the vector, the cell, or the pharmaceutical composition, as described, in the manufacture of a medicament for prophylaxis, treatment or attenuation of influenza A virus infection. In some embodiments, the antibody or antigen binding portion thereof, the nucleic acid, the vector, the cell, or the pharmaceutical composition, as described, is administered prophylactically or therapeutically.

In yet another aspect, this disclosure provides a method of reducing influenza A virus infection or lowering the risk of influenza A virus infection. The method includes administering to a subject in need thereof, a therapeutically effective amount of the antibody or antigen binding portion thereof as described above.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

In the following, a brief description of the appended figures will be given. The figures are intended to illustrate the present invention in more detail. However, they are not intended to limit the subject matter of the invention in any way.

FIGS. 1A and 1B (collectively “FIG. 1”) show the survival rates of FcγR humanized mice receiving different doses of antibodies Flu1_MLNS+GRLR (FIG. 1A) or Flu1_MLNS (FIG. 1B) four hours prior to lethal challenge with PR8 influenza virus.

FIG. 2 shows the course of the bodyweight after PR8 influenza infection for each mouse in each group (as indicated in the figure).

FIG. 3 shows the levels of Flu1_MLNS+GRLR or Flu1_MLNS in the serum of treated mice on day 4 post infection.

FIGS. 4A, 4B, and 4C (collectively “FIG. 4”) show that increasing doses of Flu1_MLNS+GAALIE administered to FcγR humanized mice prior to lethal challenge with PR8 influenza virus resulted in a dose-dependent increase in bodyweight after viral challenge (FIG. 4A), a dose-dependent increase in survival rates after viral challenge (FIG. 4B), and a dose-dependent increase in Flu1 antibody levels in the serum of treated mice (FIG. 4C).

FIGS. 5A and 5B (collectively “FIG. 5”) show the course of bodyweight (FIG. 5A) and survival rates (FIG. 5B) of FcγR humanized mice treated with Flu1 Fc variants prior to lethal challenge with influenza virus.

FIGS. 6A and 6B (collectively “FIG. 6”) show the bodyweight for the individual animals for each group (FIG. 6A) and Flu1 antibody levels for the four groups of mice receiving the distinct antibodies (FIG. 6B).

FIGS. 7A and 7B (collectively “FIG. 7”) show the bodyweights (FIG. 7A) and survival rates (FIG. 7B) for FcγR humanized mice treated with distinct Fc variants of antibody Flu1 four hours prior to infection with PR8 influenza virus.

FIGS. 8A and 8B (collectively “FIG. 8”) show Flu1 levels in the serum of treated mice three days after influenza infection (FIG. 8A) and platelet counts two days after influenza infection (FIG. 8B).

FIGS. 9A and 9B (collectively “FIG. 9”) show the bodyweights (FIG. 9A) and survival rates (FIG. 9B) for FcγR/FcRn humanized mice treated with distinct Fc variants of antibody Flu1 four hours prior to infection with PR8 influenza virus.

FIG. 10 shows the bodyweight of individual animals for each group.

FIGS. 11A and 11B (collectively “FIG. 11”) show the Flu1 antibody levels in the serum of treated mice determined on day 3 (FIG. 11A) and platelet counts on day 4 (FIG. 11B).

FIGS. 12A, 12B, and 12C (collectively “FIG. 12”) show the survival rates (FIG. 12A), bodyweights (FIG. 12B) and serum Flu1 antibody levels (determined on day of virus challenge) (FIG. 12C) for FcγR/FcRn humanized mice treated prophylactically with Flu1_wt, Flu1_MLNS, Flu1_GAALIE, Flu1_MLNS+GAALIE, or PBS five days prior to infection with PR8 influenza virus.

FIG. 13 shows the bodyweight of individual animals for each group.

FIGS. 14A and 14B (collectively “FIG. 14”) show the bodyweights (FIG. 14A) and survival rates (FIG. 14B) for FcγR/FcRn humanized mice treated prophylactically with increasing doses of Flu1_MLNS, Flu1_MLNS+GAALIE, or PBS two days prior to infection with PR8 influenza virus.

FIG. 15 shows the bodyweight of individual animals for each group.

FIG. 16 shows the serum levels of Flu1 antibodies on the day of influenza virus challenge.

FIGS. 17A and 17B (collectively “FIG. 17”) show the bodyweights (FIG. 17A) and survival rates (FIG. 17B) for FcγR humanized mice treated therapeutically with distinct Fc variants of antibody Flu1 three days after infection with PR8 influenza virus.

FIG. 18 shows the bodyweights of individual animals for each group.

FIGS. 19A and 19B (collectively “FIG. 19”) show the bodyweights (FIG. 17A) and survival rates (FIG. 17B) for FcγR humanized mice treated therapeutically with increasing doses of Flu1_wt, Flu1_GAALIE, or PBS three days after infection with PR8 influenza virus.

FIG. 20 shows the bodyweight of individual animals for each group.

FIGS. 21A, 21B, and 21C (collectively “FIG. 21”) show the FcγR binding profile of the various human IgG1 Fc domain variants (FIG. 21A), the survival rates (FIG. 21B), and the bodyweights (FIG. 21C) for FcγR humanized mice treated with distinct Fc variants of the anti-HA antibody FI6v3 (4 mg/kg, i.p.) four hours prior to infection with PR8 influenza virus.

FIGS. 22A, 22B, and 22C (collectively “FIG. 22”) show the FcγR binding profile of the various human IgG1 Fc domain variants (FIG. 22A), the survival rates (FIG. 22B), and the bodyweights (FIG. 21C) for FcγR humanized mice treated with distinct Fc variants of the anti-NA antibody 3C05 (15 mg/kg, i.p.) four hours prior to infection with Neth09 H1N1 influenza virus.

FIGS. 23A, 23B, 23C, 23D, and 23E (collectively “FIG. 23”) show the FcγR binding profile of the various human IgG1 Fc domain variants (FIG. 23A), the survival rates (FIG. 23B and FIG. 23D), and the bodyweights (FIG. 23C and FIG. 23E) for FcγR humanized mice treated with distinct Fc variants of the anti-M2e antibody TCN032 (10 mg/kg, i.v. for FIGS. 23B-C; 2 or 5 mg/kg for FIGS. 23D-E) four hours prior to infection with PR8 influenza virus.

FIGS. 24A, 24B, 24C, 24D, and 24E (collectively “FIG. 24”) show the FcγR binding profile of the various human IgG1 Fc domain variants (FIG. 24A), the survival rates (FIG. 24B and FIG. 24D), and the bodyweights (FIG. 24C and FIG. 24E) for FcγR humanized mice treated with distinct Fc variants of the anti-M2e antibody 14C2 (10 mg/kg, i.v. for FIGS. 24B-C; 2 or 5 mg/kg for FIGS. 24D-E) four hours prior to infection with PR8 influenza virus.

FIGS. 25A, 25B, and 25C (collectively “FIG. 25”) show the survival rates (FIG. 25A), and the bodyweights (FIG. 25B) for FcγR humanized mice treated with distinct Fc variants of the neutralizing anti-HA head antibody 4G05 (0.5 mg/kg, i.v.) four hours prior to infection with Neth09 H1N1 influenza virus (5 mLD50 i.n.). FIG. 25C shows the serum levels of 4G05 mAb on day 4 post-infection.

FIGS. 26A, 26B, and 26C (collectively “FIG. 26”) show the bodyweights (FIG. 26A), and the survival rate (FIG. 26B) for FcγR humanized mice treated with distinct Fc variants of the non-neutralizing anti-HA head antibody 1A01 (2 mg/kg, i.v.) four hours prior to infection with Neth09 H1N1 influenza virus (5 mLD50 i.n.). FIG. 26C shows the serum levels of 1A01 mAb on day 4 post-infection.

FIGS. 27A and 27B (collectively “FIG. 27”) show the percentage of mature DCs (defined as CD86hi/CD80hi; FIG. 27A) and activated CD4 and CD8 T cells (defined as CD44+CD69+; FIG. 27B) present on day 4 post-infection in the lungs of FcγR humanized mice treated with distinct Fc variants of the anti-HA stalk antibody FI6v3 (3 mg/kg, i.p.) four hours prior to infection with PR8 H1N1 influenza virus (5 mLD50 i.n.).

FIGS. 28A and 28B (collectively “FIG. 28”) show abundance and FcγR expression profile of DC populations in the lungs of influenza-infected FcγR humanized mice at different time points following infection. To determine the abundance and FcγR expression profile of DC subsets during the course of influenza infection, cohorts of FcγR humanized mice were infected (i.n. with H1N1 PR8; 5 mLD50) and euthanized at different time points following infection (day 0 to day 6). Lungs were homogenized and analyzed by flow cytometry to determine the frequency (FIG. 28A) and FcγR expression profile (FIG. 28B) of the three major DC subsets identified: cDC1 (defined as MHCII/CD11c+/CD11b−/CD103+), cDC2 (defined as MHCII/CD11c+/CD11b+/CD103−/Gr-1−), and tipDC (TNF-α/iNOS-producing DCs defined as MHCII/CD11c+/CD11b+/CD103−/Gr-1+). Influenza infection was not associated with any major changes in the number of lung-resident cDC1 and cDC2, whereas tipDCs were almost absent at baseline, but their number increased dramatically upon infection. cDC1 and cDC2 expressed FcγRIIa and FcγRIIb, but they were negative for FcγRIIIa. In contrast, tipDCs expressed FcγRIIa and FcγRIIIa, along with the inhibitory FcγRIIb. Due to the very low number of tipDCs at baseline, FcγR expression (MFI) was omitted from the heatmap plot. n=4 mice/time point assessed.

FIGS. 29A and 29B (collectively “FIG. 29”) show treatment of FcγR humanized mice with GAALIE variants of anti-HA mAbs is associated with increased frequency of activated DCs. To investigate the impact of enhanced FcγRIIa engagement by GAALIE variants on the maturation status of DCs, FcγR humanized mice were treated with Fc domain variants of the anti-HA stalk mAb FI6v3, exhibiting differential FcγR affinity—wild type IgG1 (baseline FcγR affinity), GRLR (diminished binding to all classes of FcγRs), and GAALIE (enhanced FcγRIIa and FcγRIIIa affinity). Fc domain variants were administered i.p. (3 mg/kg) to FcγR humanized mice 4 h prior to lethal challenge with H1N1 (PR8; 5 mLD50). Mice were euthanized on day 4 and lung-resident DCs were analyzed by flow cytometry. The abundance of mature (defined as CD80high/CD86high) cDC1 (FIG. 29A) and cDC2 (FIG. 29B) was compared between mice treated with the various Fc domain variants of FI6v3. Representative flow cytometry plots from data presented in FIG. 27A.

FIGS. 30A, 30B, 30C, and 30D (collectively “FIG. 30”) show the survival rates (FIG. 30A), and the bodyweights (FIG. 30B) for FcγR humanized mice treated with distinct Fc variants of the anti-HA antibody Flu_1 (2 mg/kg, i.p.) four hours prior to infection with PR8 H1N1 influenza virus (5 mLD50 i.n.). Isotype (rat IgG2b; clone LTF-2) or anti-mouse CD8 (clone 2.43) was administered to mice (150 μg i.p.) on day 3 post-infection. FIG. 30C shows the serum levels of Flu_1 mAb on day 4 post-infection. FIG. 30D shows the frequency of CD8 T cells in the blood of FcγR humanized mice treated with isotype (rat IgG2b; clone LTF-2) or anti-mouse CD8 (clone 2.43).

FIGS. 31A and 31B (collectively “FIG. 31”) show treatment of FcγR humanized mice with GAALIE variants of anti-HA stalk mAbs is associated with enhanced activation of CD8+ and CD4+ T cells. To investigate whether the observed increase in the frequency of mature DCs in mice treated with GAALIE variants of antiHA mAbs was associated with enhanced T cell responses, the activation status of CD8 and CD4 T cells was analyzed and compared between mice treated with anti-HA Fc domain variants with differential FcγR affinity (wild type IgG1, GRLR, and GAALIE). Fc domain variants of the antiHA stalk mAb FI6v3 were administered (i.p. 3 mg/kg) to FcγR humanized mice prior to lethal challenge with H1N1 (PR8; 5 mLD50). Mice were euthanized on day 4 post-infection and T-cell populations were analyzed by multicolor flow cytometry. The frequency of activated (defined as CD44hi/CD69+) CD8+(FIG. 31A) and CD4+(FIG. 31B) T cells was compared between mice treated with the various Fc domain variants of FI6v3. Representative flow cytometry plots from data presented in FIG. 27B.

### DETAILED DESCRIPTION OF THE INVENTION

Antibodies against viral pathogens represent promising therapeutic modalities for the control of infection and several studies have previously established that their antiviral efficacy requires the coordinated function of both Fab and Fc domains¹. The Fc domain engages a wide spectrum of receptors (FcγRs) on discrete cells of the immune system to trigger the clearance of virus and killing of infected cells¹⁻⁴⁰. This disclosure demonstrated that Fc engineering of antibodies, such as anti-influenza IgG monoclonal antibodies (mAbs), for selective binding to the dendritic cell FcγR, FcγRIIa, results in enhanced protection from, and treatment of, a lethal viral respiratory infection through the induction of protective CD8⁺ T-cell responses. These findings highlight the capacity to IgG antibodies to induce protective adaptive immunity to viral infection when they selectively activate a dendritic cell—T-cell pathway, having important implications for the development of antibody therapeutics with improved antiviral efficacy against viral respiratory pathogens, like influenza and SARS-CoV-2.

### A. Antibodies

The invention is based, amongst other findings, on the identification of antibodies that reduce viral infection, such as influenza A infection, and exhibit enhanced efficacy. One of the crucial mechanisms of action of a therapeutic antibody is the targeted elimination of viruses and/or infected cells through recruitment of the immune system. This is typically achieved by interaction of the antibody's Fc domain with Fcγ receptors (FcγRs; FcgammaRs; FcgRs) and/or the complement component C1q. Antibodies of the present invention show increased effector functions, namely, an enhanced ability to mediate cellular cytotoxicity functions, such as antibody-dependent cell-mediated cytotoxicity (ADCC) and antibody-dependent cell-mediated phagocytosis (ADCP).

In one aspect, this disclosure provides an isolated Fc receptor-dependent antibody or antigen binding portion thereof capable of activating dendritic cell maturation.

In another aspect, this disclosure provides an isolated Fc receptor-dependent antibody or antigen binding portion thereof capable of inducing a protective CD8 response.

In some embodiments, the antibody or antigen binding portion thereof binds specifically to a viral antigen. In some embodiments, the viral antigen comprises an influenza virus antigen comprising hemagglutinin (HA) or neuraminidase (NA).

In some embodiments, the antibody or antigen binding portion thereof comprises (i) a heavy chain having a G236A mutation in a constant region thereof and (ii) an Fc region, wherein the Fc region activates FcγRIIa.

In a first aspect, the present invention provides an (isolated) antibody or antigen binding portion thereof comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutation G236A in the constant region of the heavy chain.

In some embodiments, the antibody or antigen binding portion thereof comprises the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 11, and SEQ ID NO: 6, respectively.

In some embodiments, the antibody or antigen binding portion thereof comprises the heavy chain CDR1, CDR2, and CDR3 sequences as set forth in SEQ ID NO: 26, SEQ ID NO: 27, and SEQ ID NO: 28, respectively; and the light chain CDR1, CDR2, and CDR3 sequences as set forth in: SEQ ID NO: 29, SEQ ID NO: 30, and SEQ ID NO: 31, respectively.

In some embodiments, the antibody or antigen binding portion thereof comprises the heavy chain CDR1, CDR2, and CDR3 sequences as set forth in SEQ ID NO: 36, SEQ ID NO: 37, and SEQ ID NO: 38, respectively; and the light chain CDR1, CDR2, and CDR3 sequences as set forth in: SEQ ID NO: 39, SEQ ID NO: 40, and SEQ ID NO: 41, respectively.

In some embodiments, the antibody or antigen binding portion thereof comprises the heavy chain CDR1, CDR2, and CDR3 sequences as set forth in SEQ ID NO: 46, SEQ ID NO: 47, and SEQ ID NO: 48, respectively; and the light chain CDR1, CDR2, and CDR3 sequences as set forth in: SEQ ID NO: 49, SEQ ID NO: 50, and SEQ ID NO: 51, respectively.

In some embodiments, the antibody or antigen binding portion thereof comprises the heavy chain CDR1, CDR2, and CDR3 sequences as set forth in SEQ ID NO: 56, SEQ ID NO: 57, and SEQ ID NO: 58, respectively; and the light chain CDR1, CDR2, and CDR3 sequences as set forth in: SEQ ID NO: 59, SEQ ID NO: 60, and SEQ ID NO: 61, respectively.

In addition to the mutation G236A in the constant region of the heavy chain, the antibody may or may not comprise the mutations A330L and I332E in the constant region of the heavy chain. In some embodiments, the antibody further comprises the mutations A330L and I332E.

In some embodiments, the antibody does not comprise the mutation S239D in the constant region of the heavy chain.

In general, the antibody according to the present invention, typically comprises (at least) three complementarity determining regions (CDRs) on a heavy chain and (at least) three CDRs on a light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e., heavy and light chain), there are six CDRs for each antigen receptor (heavy chain: CDRH1, CDRH2, and CDRH3; light chain: CDRL1, CDRL2, and CDRL3). A single antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a chain (or each chain, respectively) may be composed of four framework regions, separated by three CDRs.

The sequences of the heavy chains and light chains of exemplary antibodies of the invention, comprising three different CDRs on the heavy chain and three different CDRs on the light chain were determined. The position of the CDR amino acids is defined according to the IMGT numbering system (IMGT: http://www.imgt.org/; cf. Lefranc, M.-P. et al. (2009) Nucleic Acids Res. 37, D1006-D1012).

Typically, the antibody of the invention binds to hemagglutinin of an influenza A virus. Thereby, the antibody of the invention can neutralize infection of influenza A virus. By virtue of the six CDR sequences as defined above, the antibody according to the present invention binds to the same epitope of the influenza A virus hemagglutinin (IAV HA) stem region as antibody FY1 (Kallewaard N L, Corti D, Collins P J, et al. Structure and Function Analysis of an Antibody Recognizing All Influenza A Subtypes. Cell. 2016; 166(3):596-608), thereby providing the same broad protection against various influenza A serotypes of all influenza A subtypes.

To study and quantitate virus infectivity (or “neutralization”) in the laboratory, the person skilled in the art knows various standard “neutralization assays.” For a neutralization assay, animal viruses are typically propagated in cells and/or cell lines. For example, in a neutralization assay, cultured cells may be incubated with a fixed amount of influenza A virus (IAV) in the presence (or absence) of the antibody to be tested. As a readout, for example, flow cytometry may be used. Alternatively, also other readouts are conceivable.

The antibody of the present invention includes the mutation G236A in the constant region of the heavy chain (in the CH2 region). As outlined above, the antibody may further comprise the mutations A330L and I332E in the constant region of the heavy chain (in the CH2 region). In some embodiments, the antibody does not comprise the mutation S239D in the constant region of the heavy chain. In the context of the constant region of an antibody, the amino acid positions have been numbered herein according to the art-recognized EU numbering system. The EU index or EU index as in Kabat or EU numbering refers to the numbering of the EU antibody (Edelman G M, et al. Proc Natl Acad Sci USA. 1969; 63(1): 78-85; Kabat E. A., National Institutes of Health (U. S.) Office of the Director, “Sequences of Proteins of Immunological Interest,” 5^{th} edition, Bethesda, Md.: U.S. Dept. of Health and Human Services, Public Health Service, National Institutes of Health, 1991, hereby entirely incorporated by reference). As shown in the enclosed Examples, the mutation G236A and the three mutations G236A, A330L, and I332E result in increased effector functions of the antibody, which result in increased protection against influenza infection.

Furthermore, the present invention provides an (isolated) antibody or antigen binding portion thereof may include the mutations A330L and/or I332E in the constant region of the heavy chain. In addition, the antibody or antigen binding portion thereof may or may not comprise the mutation G236A in the constant region of the heavy chain. For example, the antibody or antigen binding portion thereof comprises the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations A330L and/or I332E in the constant region of the heavy chain. In some embodiments, the antibody or antigen binding portion thereof comprises the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 11, and SEQ ID NO: 6, respectively; and the mutations A330L and/or I332E in the constant region of the heavy chain.

In some embodiments, the antibody or antigen binding portion thereof comprises the heavy chain CDR1, CDR2, and CDR3 sequences as set forth in SEQ ID NO: 26, SEQ ID NO: 27, and SEQ ID NO: 28, respectively; and the light chain CDR1, CDR2, and CDR3 sequences as set forth in: SEQ ID NO: 29, SEQ ID NO: 30, and SEQ ID NO: 31, respectively; and the mutations A330L and/or I332E in the constant region of the heavy chain.

In some embodiments, the antibody or antigen binding portion thereof comprises the heavy chain CDR1, CDR2, and CDR3 sequences as set forth in SEQ ID NO: 36, SEQ ID NO: 37, and SEQ ID NO: 38, respectively; and the light chain CDR1, CDR2, and CDR3 sequences as set forth in: SEQ ID NO: 39, SEQ ID NO: 40, and SEQ ID NO: 41, respectively; and the mutations A330L and/or I332E in the constant region of the heavy chain.

In some embodiments, the antibody or antigen binding portion thereof comprises the heavy chain CDR1, CDR2, and CDR3 sequences as set forth in SEQ ID NO: 56, SEQ ID NO: 57, and SEQ ID NO: 58, respectively; and the light chain CDR1, CDR2, and CDR3 sequences as set forth in: SEQ ID NO: 59, SEQ ID NO: 60, and SEQ ID NO: 61, respectively; and the mutations A330L and/or I332E in the constant region of the heavy chain.

In some embodiments, the antibody or antigen binding portion thereof comprises the heavy chain CDR1, CDR2, and CDR3 sequences as set forth in SEQ ID NO: 46, SEQ ID NO: 47, and SEQ ID NO: 48, respectively; and the light chain CDR1, CDR2, and CDR3 sequences as set forth in: SEQ ID NO: 49, SEQ ID NO: 50, and SEQ ID NO: 51, respectively; and the mutations A330L and/or I332E in the constant region of the heavy chain.

In some embodiments, the antibody also comprises a half-life increasing mutation in the constant region of the heavy chain. In general, the expression “half-life increasing mutation” may refer to a single mutation, such as a single amino acid substitution, or a group of mutations, such as a group of (i.e., more than one, e.g., 2, 3, 4, 5, 6, 7, 8, 9, 10 or more) amino acid substitutions, which mediate increased half-life of the antibody. Examples of such modifications include, but are not limited to, substitutions of at least one amino acid from the heavy chain constant region selected from the group consisting of amino acid residues 250, 314, and 428. Further examples of such half-life extending Fc modifications are described in Wang Y, et al. 2014 May; 22(4):269-78, which is incorporated herein by reference. In some embodiments, the antibody comprises the mutation(s) M428L and/or N434S in the heavy chain constant region (CH3 region). In particular, the mutations G236A, A330L, and I332E in the constant region of the heavy chain of the antibody of the invention do not compromise the half-life increasing effect of respective mutations in the constant region, as shown in the enclosed Examples.

The present invention also provides an (isolated) antibody or antigen binding portion thereof comprising the mutations M428L and/or N434S in the constant region of the heavy chain. In addition, the antibody may or may not comprise one, two or all of the mutations G236A, A330L, and I332E in the constant region of the heavy chain. For example, the antibody or antigen binding portion thereof comprises the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations M428L and/or N434S in the constant region of the heavy chain. In some embodiments, the antibody of the invention comprises the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 11, and SEQ ID NO: 6, respectively; and the mutations M428L and/or N434S in the constant region of the heavy chain.

In some embodiments, the antibody or antigen binding portion thereof comprises the heavy chain CDR1, CDR2, and CDR3 sequences as set forth in SEQ ID NO: 26, SEQ ID NO: 27, and SEQ ID NO: 28, respectively; and the light chain CDR1, CDR2, and CDR3 sequences as set forth in: SEQ ID NO: 29, SEQ ID NO: 30, and SEQ ID NO: 31, respectively; and the mutations M428L and/or N434S in the constant region of the heavy chain.

In some embodiments, the antibody or antigen binding portion thereof comprises the heavy chain CDR1, CDR2, and CDR3 sequences as set forth in SEQ ID NO: 36, SEQ ID NO: 37, and SEQ ID NO: 38, respectively; and the light chain CDR1, CDR2, and CDR3 sequences as set forth in: SEQ ID NO: 39, SEQ ID NO: 40, and SEQ ID NO: 41, respectively; and the mutations M428L and/or N434S in the constant region of the heavy chain.

In some embodiments, the antibody or antigen binding portion thereof comprises the heavy chain CDR1, CDR2, and CDR3 sequences as set forth in SEQ ID NO: 56, SEQ ID NO: 57, and SEQ ID NO: 58, respectively; and the light chain CDR1, CDR2, and CDR3 sequences as set forth in: SEQ ID NO: 59, SEQ ID NO: 60, and SEQ ID NO: 61, respectively; and the mutations M428L and/or N434S in the constant region of the heavy chain.

In some embodiments, the antibody or antigen binding portion thereof comprises the heavy chain CDR1, CDR2, and CDR3 sequences as set forth in SEQ ID NO: 46, SEQ ID NO: 47, and SEQ ID NO: 48, respectively; and the light chain CDR1, CDR2, and CDR3 sequences as set forth in: SEQ ID NO: 49, SEQ ID NO: 50, and SEQ ID NO: 51, respectively; and the mutations M428L and/or N434S in the constant region of the heavy chain.

Antibodies of the invention may be low fucosylated or afucosylated. An afucosylated antibody is engineered such, that the oligosaccharides in the Fc region of the antibody do not have any fucose sugar units (or a decreased number of fucose in low fucosylated antibodies). Afucosylated antibodies can be obtained by techniques known in the art, for example, by using engineered CHO cells, which can express afucosylated antibodies. Various strategies to produce afucosylated antibodies are described in: Pereira N A et al. MAbs.; 10(5): 693-711, which is incorporated herein by reference. In some embodiments, the antibody of the invention (i) comprises the mutations M428L and N434S (but not the mutations G236A, A330L, and 1332E); and (ii) is afucosylated.

Antibodies of the invention do usually not comprise the mutations G236R and L328R in the constant region of the heavy chain. Moreover, the antibody does typically not comprise the mutations G237D, P238D, H268D, P271G, and A330R in the constant regions of the heavy chain.

In some embodiments, the antibody of the invention is a human antibody. In some embodiments, the antibody of the invention is a monoclonal antibody. For example, the antibody of the invention is a human monoclonal antibody.

Antibodies of the invention can be of any isotype (e.g., IgA, IgG, IgM, i.e., an α, γ or μ heavy chain). For example, the antibody is of the IgG type. Within the IgG isotype, antibodies may be IgG1, IgG2, IgG3 or IgG4 subclass, for example, IgG1. Antibodies of the invention may have a κ or a λ light chain. In some embodiments, the antibody has a kappa (κ) light chain. In some embodiments, the antibody is of IgG1 type and has a κ light chain.

In some embodiments, the antibody is of the human IgG1 type. The antibody may be of any allotype. The term “allotype” refers to the allelic variation found among the IgG subclasses. For example, the antibody may be of the Glm1 (or Glm(a)) allotype, of the Glm2 (or Glm(x)) allotype, of the Glm3 (or Glm(f)) allotype, and/or of the G1m17 (or Gm(z)) allotype. The Glm3 and Glm17 allotypes are located at the same position in the CH1 domain (position 214, according to EU numbering). G1m3 corresponds to R214 (EU), while G1m17 corresponds to K214 (EU). The Glm1 allotype is located in the CH3 domain (at positions 356 and 358 (EU)) and refers to the replacements E356D and M358L. The Glm2 allotype refers to a replacement of the alanine in position 431 (EU) by a glycine. The Glm1 allotype may be combined, for example, with the Glm3 or the G1m17 allotype. In some embodiments, the antibody is of the allotype G1m3 with no Glm1 (G1m3,−1). In some embodiments, the antibody is of the Glm17,1 allotype. In some embodiments, the antibody is of the G1m3,1 allotype. In some embodiments, the antibody is of the allotype G1m17 with no Glm1 (G1m17,−1). Optionally, these allotypes may be combined (or not combined) with the Glm2, G1m27 or G1m28 allotype. For example, the antibody may be of the G1m17,1,2 allotype.

In some embodiments, the antibody of the invention or antigen binding portion thereof comprises a heavy chain variable region comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention comprises a heavy chain variable region comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 11, and SEQ ID NO: 6, respectively) are maintained.

In some embodiments, the antibody of the invention or antigen binding portion thereof comprises a heavy chain variable region comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 32 and a light chain variable region comprising the amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 33, wherein the CDR sequences as defined above (the heavy chain CDR1, CDR2, and CDR3 sequences as set forth in SEQ ID NO: 26, SEQ ID NO: 27, and SEQ ID NO: 28, respectively, and the light chain CDR1, CDR2, and CDR3 sequences as set forth in: SEQ ID NO: 29, SEQ ID NO: 30, and SEQ ID NO: 31, respectively;) are maintained.

In some embodiments, the antibody of the invention or antigen binding portion thereof comprises a heavy chain variable region comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 42 and a light chain variable region comprising the amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 43, wherein the CDR sequences as defined above the heavy chain CDR1, CDR2, and CDR3 sequences as set forth in SEQ ID NO: 36, SEQ ID NO: 37, and SEQ ID NO: 38, respectively, and the light chain CDR1, CDR2, and CDR3 sequences as set forth in: SEQ ID NO: 39, SEQ ID NO: 40, and SEQ ID NO: 41, respectively;) are maintained.

In some embodiments, the antibody of the invention or antigen binding portion thereof comprises a heavy chain variable region comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 52 and a light chain variable region comprising the amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 53, wherein the CDR sequences as defined above (the heavy chain CDR1, CDR2, and CDR3 sequences as set forth in SEQ ID NO: 46, SEQ ID NO: 47, and SEQ ID NO: 48, respectively, and the light chain CDR1, CDR2, and CDR3 sequences as set forth in: SEQ ID NO: 49, SEQ ID NO: 50, and SEQ ID NO: 51, respectively;) are maintained.

In some embodiments, the antibody of the invention or antigen binding portion thereof comprises a heavy chain variable region comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 62 and a light chain variable region comprising the amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 63, wherein the CDR sequences as defined above (the heavy chain CDR1, CDR2, and CDR3 sequences as set forth in SEQ ID NO: 56, SEQ ID NO: 57, and SEQ ID NO: 58, respectively, and the light chain CDR1, CDR2, and CDR3 sequences as set forth in: SEQ ID NO: 59, SEQ ID NO: 60, and SEQ ID NO: 61, respectively;) are maintained.

Sequence identity is usually calculated with regard to the full length of the reference sequence (i.e., the sequence recited in the application). Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=11 and gap extension penalty=1].

A “sequence variant” has an altered sequence in which one or more of the amino acids in the reference sequence is/are deleted or substituted, and/or one or more amino acids is/are inserted into the sequence of the reference amino acid sequence. As a result of the alterations, the amino acid sequence variant has an amino acid sequence which is at least 70% identical to the reference sequence. Variant sequences which are at least 70% identical have no more than 30 alterations, i.e., any combination of deletions, insertions or substitutions, per 100 amino acids of the reference sequence.

In general, while it is possible to have non-conservative amino acid substitutions, the substitutions are usually conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine, and isoleucine, with another; substitution of one hydroxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine, and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

In some embodiments, The antibody or antigen binding portion thereof of any one of the preceding claims, wherein the antibody or antigen binding portion thereof comprises: (i) a heavy chain variable region comprising an amino acid sequence having at least 75% identity (e.g., at least 75% identity, at least 80% identity, at least 85% identity, at least 90% identity, at least 95% identity) to SEQ ID NO: 7 and a light chain variable region comprising the amino acid sequence having at least 75% identity (e.g., at least 75% identity, at least 80% identity, at least 85% identity, at least 90% identity, at least 95% identity) to SEQ ID NO: 8; (ii) a heavy chain variable region comprising an amino acid sequence having at least 75% identity (e.g., at least 75% identity, at least 80% identity, at least 85% identity, at least 90% identity, at least 95% identity) to SEQ ID NO: 32 and a light chain variable region comprising the amino acid sequence having at least 75% identity (e.g., at least 75% identity, at least 80% identity, at least 85% identity, at least 90% identity, at least 95% identity) to SEQ ID NO: 33; (iii) a heavy chain variable region comprising an amino acid sequence having at least 75% identity (e.g., at least 75% identity, at least 80% identity, at least 85% identity, at least 90% identity, at least 95% identity) to SEQ ID NO: 42 and a light chain variable region comprising the amino acid sequence having at least 75% identity (e.g., at least 75% identity, at least 80% identity, at least 85% identity, at least 90% identity, at least 95% identity) to SEQ ID NO: 43; (iv) a heavy chain variable region comprising an amino acid sequence having at least 75% identity to SEQ ID NO: 52 and a light chain variable region comprising the amino acid sequence having at least 75% identity (e.g., at least 75% identity, at least 80% identity, at least 85% identity, at least 90% identity, at least 95% identity) to SEQ ID NO: 53; or (v) a heavy chain variable region comprising an amino acid sequence having at least 75% identity (e.g., at least 75% identity, at least 80% identity, at least 85% identity, at least 90% identity, at least 95% identity) to SEQ ID NO: 62 and a light chain variable region comprising the amino acid sequence having at least 75% identity (e.g., at least 75% identity, at least 80% identity, at least 85% identity, at least 90% identity, at least 95% identity) to SEQ ID NO: 63.

In some embodiments, the antibody of the invention or antigen binding portion thereof comprises a heavy chain comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 9 and a light chain comprising the amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 10, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively, or set forth in SEQ ID NO: 4, SEQ ID NO: 11, and SEQ ID NO: 6, respectively) are maintained.

In some embodiments, the antibody of the invention or antigen binding portion thereof comprises a heavy chain comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 34 and a light chain comprising the amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 35, wherein the CDR sequences as defined above (the heavy chain CDR1, CDR2, and CDR3 sequences as set forth in SEQ ID NO: 26, SEQ ID NO: 27, and SEQ ID NO: 28, respectively, and the light chain CDR1, CDR2, and CDR3 sequences as set forth in: SEQ ID NO: 29, SEQ ID NO: 30, and SEQ ID NO: 31, respectively;) are maintained.

In some embodiments, the antibody of the invention or antigen binding portion thereof comprises a heavy chain comprising an amino acid sequence having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 44 and a light chain comprising the amino acid sequence having at least 70% (e.g., 71%, 72%), 73%, 74%, 75% 76%, 77% 78%, 79%, 80%), 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 45, wherein the CDR sequences as defined above the heavy chain CDR1, CDR2, and CDR3 sequences as set forth in SEQ ID NO: 36, SEQ ID NO: 37, and SEQ ID NO: 38, respectively, and the light chain CDR1, CDR2, and CDR3 sequences as set forth in: SEQ ID NO: 39, SEQ ID NO: 40, and SEQ ID NO: 41, respectively;) are maintained.

In some embodiments, the antibody of the invention or antigen binding portion thereof comprises a heavy chain comprising an amino acid sequence having 70% or more (e.g., 71%, 72%, 73% 74%, 75%, 76%, 77% 78%, 79% 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 54 and a light chain comprising the amino acid sequence having at least 70% (e.g., 71%, 72%), 73%, 74%, 75% 76%, 77%, 78%, 79% 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 55, wherein the CDR sequences as defined above (the heavy chain CDR1, CDR2, and CDR3 sequences as set forth in SEQ ID NO: 46, SEQ ID NO: 47, and SEQ ID NO: 48, respectively, and the light chain CDR1, CDR2, and CDR3 sequences as set forth in: SEQ ID NO: 49, SEQ ID NO: 50, and SEQ ID NO: 51, respectively;) are maintained.

In some embodiments, the antibody of the invention or antigen binding portion thereof comprises a heavy chain comprising an amino acid sequence having 70% or more (e.g., 71%, 72%, 73%, 74%, 75%, 76%, 77% 78%, 79% 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 64 and a light chain comprising the amino acid sequence having at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 65, wherein the CDR sequences as defined above (the heavy chain CDR1, CDR2, and CDR3 sequences as set forth in SEQ ID NO: 56, SEQ ID NO: 57, and SEQ ID NO: 58, respectively, and the light chain CDR1, CDR2, and CDR3 sequences as set forth in: SEQ ID NO: 59, SEQ ID NO: 60, and SEQ ID NO: 61, respectively;) are maintained.

In some embodiments, the antibody or antigen binding portion thereof comprises: (i) the heavy chain CDR1, CDR2, and CDR3 sequences as set forth in SEQ ID NO: 1, SEQ ID NO: 2, and SEQ ID NO: 3, respectively, and the light chain CDR1, CDR2, and CDR3 sequences as set forth in: SEQ ID NO: 4, SEQ ID NO: 5, and SEQ ID NO: 6, respectively; and a heavy chain variable region comprising an amino acid sequence as set forth in SEQ ID NO: 7 and a light chain variable region comprising the amino acid sequence as set forth in SEQ ID NO: 8; (ii) the heavy chain CDR1, CDR2, and CDR3 sequences as set forth in SEQ ID NO: 26, SEQ ID NO: 27, and SEQ ID NO: 28, respectively, and the light chain CDR1, CDR2, and CDR3 sequences as set forth in: SEQ ID NO: 29, SEQ ID NO: 30, and SEQ ID NO: 31, respectively; and a heavy chain variable region comprising an amino acid sequence set forth in SEQ ID NO: 32 and a light chain variable region comprising the amino acid sequence set forth in SEQ ID NO: 33; (iii) the heavy chain CDR1, CDR2, and CDR3 sequences as set forth in SEQ ID NO: 36, SEQ ID NO: 37, and SEQ ID NO: 38, respectively, and the light chain CDR1, CDR2, and CDR3 sequences as set forth in: SEQ ID NO: 39, SEQ ID NO: 40, and SEQ ID NO: 41, respectively; and a heavy chain variable region comprising an amino acid sequence set forth in SEQ ID NO: 42 and a light chain variable region comprising the amino acid sequence set forth in SEQ ID NO: 43; (iv) the heavy chain CDR1, CDR2, and CDR3 sequences as set forth in SEQ ID NO: 46, SEQ ID NO: 47, and SEQ ID NO: 48, respectively, and the light chain CDR1, CDR2, and CDR3 sequences as set forth in: SEQ ID NO: 49, SEQ ID NO: 50, and SEQ ID NO: 51, respectively; and a heavy chain variable region comprising an amino acid sequence set forth in SEQ ID NO: 52 and a light chain variable region comprising the amino acid sequence set forth in SEQ ID NO: 53; or (v) the heavy chain CDR1, CDR2, and CDR3 sequences as set forth in SEQ ID NO: 56, SEQ ID NO: 57, and SEQ ID NO: 58, respectively, and the light chain CDR1, CDR2, and CDR3 sequences as set forth in: SEQ ID NO: 59, SEQ ID NO: 60, and SEQ ID NO: 61, respectively; and a heavy chain variable region comprising an amino acid sequence set forth in SEQ ID NO: 62 and a light chain variable region comprising the amino acid sequence set forth in SEQ ID NO: 63.

In general, it is possible that the antibody of the invention comprises one or more further mutations (in addition to the mutation G236A (and A330L and I332E) and, optionally, a half-life increasing mutation, such as M428L and N4345) in the Fc region (e.g., in the CH2 or CH3 region). However, in some embodiments, the antibody of the invention does not comprise any further mutation in addition to G236A, A330L, and I332E in its CH2 region (in comparison to the respective wild-type CH2 region). In some embodiments, the antibody of the invention does not comprise any further mutation in addition to G236A in its CH2 region (in comparison to the respective wild-type CH2 region).

In some embodiments, the antibody of the invention does not comprise any further mutation in addition to M428L and N434S in its CH3 region (in comparison to the respective wild-type CH3 region).

In some embodiments, the antibody of the invention does not comprise (i) any mutation in its CH3 region; or (ii) any further mutation in addition to M428L and N434S in its CH3 region (in comparison to the respective wild-type CH3 region). In some embodiments, the antibody of the invention does not comprise any further mutation in addition to G236A, A330L, and I332E and, optionally, M428L and N434S, in its Fc region (in comparison to the respective wild-type Fc region). As used herein, the term “wild-type” refers to the reference sequence, for example as occurring in nature. As a specific example, the term “wild-type” may refer to the sequence with the highest prevalence occurring in nature. In some embodiments, the antibody of the invention does not comprise any further mutation in addition to M428L and N434S in its Fc region (in comparison to the respective wild-type Fc region).

In some embodiments, the antibody or antigen binding portion thereof comprises a light chain comprising an amino acid sequence as set forth in SEQ ID NO: 10 and a heavy chain comprising an amino acid sequence as set forth in SEQ ID NOs: 9, 13, 14, 18, or 19. In some embodiments, the antibody or antigen binding portion thereof comprises a light chain comprising an amino acid sequence as set forth in SEQ ID NO: 35 and a heavy chain comprising an amino acid sequence as set forth in SEQ ID NOs: 66, 68, 69 or 70. In some embodiments, the antibody or antigen binding portion thereof comprises a light chain comprising an amino acid sequence as set forth in SEQ ID NO: 45 and a heavy chain comprising an amino acid sequence as set forth in SEQ ID NOs: 73, 74 or 75. In some embodiments, the antibody or antigen binding portion thereof comprises a light chain comprising an amino acid sequence as set forth in SEQ ID NO: 55 and a heavy chain comprising an amino acid sequence as set forth in SEQ ID NOs: 77, 78 or 79. In some embodiments, the antibody or antigen binding portion thereof comprises a light chain comprising an amino acid sequence as set forth in SEQ ID NO: 65 and a heavy chain comprising an amino acid sequence as set forth in SEQ ID NOs: 81, 82, 83 or 84.

In some embodiments, the antibody or antigen binding portion thereof comprises a light chain with an amino acid sequence as set forth in SEQ ID NO: 10 and a heavy chain with an amino acid sequence as set forth in SEQ ID NOs: 9, 13, 14, 18, or 19. In some embodiments, the antibody or antigen binding portion thereof comprises a light chain with an amino acid sequence as set forth in SEQ ID NO: 35 and a heavy chain with an amino acid sequence as set forth in SEQ ID NOs: 66, 68, 69 or 70. In some embodiments, the antibody or antigen binding portion thereof comprises a light chain with an amino acid sequence as set forth in SEQ ID NO: 45 and a heavy chain with an amino acid sequence as set forth in SEQ ID NOs: 73, 74 or 75. In some embodiments, the antibody or antigen binding portion thereof comprises a light chain with an amino acid sequence as set forth in SEQ ID NO: 55 and a heavy chain with an amino acid sequence as set forth in SEQ ID NOs: 77, 78 or 79. In some embodiments, the antibody or antigen binding portion thereof comprises a light chain with an amino acid sequence as set forth in SEQ ID NO: 65 and a heavy chain with an amino acid sequence as set forth in SEQ ID NOs: 81, 82, 83 or 84.

Antibodies of the invention also include hybrid antibody molecules that comprise the six CDRs from an antibody of the invention as defined above and one or more CDRs from another antibody to the same or a different epitope or antigen. In some embodiments, such hybrid antibodies comprise six CDRs from an antibody of the invention and six CDRs from another antibody to a different epitope or antigen.

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Antibodies of the invention may be provided in purified form. Typically, the antibody will be present in a composition that is substantially free of other polypeptides, e.g., where less than 90% (by weight), usually less than 60% and more usually less than 50% of the composition is made up of other polypeptides.

Antibodies of the invention may be immunogenic in nonhuman (or heterologous) hosts, e.g., in mice. In particular, the antibodies may have an idiotope that is immunogenic in nonhuman hosts, but not in a human host. In particular, antibodies of the invention for human use include those that cannot be easily isolated from hosts such as mice, goats, rabbits, rats, non-primate mammals, etc. and cannot generally be obtained by humanization or from xeno-mice.

### B. Nucleic Acids

In another aspect, the invention also provides a nucleic acid molecule comprising a polynucleotide encoding the antibody according to the present invention, as described above. Examples of nucleic acid molecules and/or polynucleotides include, e.g., a recombinant polynucleotide, a vector, an oligonucleotide, an RNA molecule such as an rRNA, an mRNA, an miRNA, a siRNA, or a tRNA, or a DNA molecule such as a cDNA. Nucleic acids may encode the light chain and/or the heavy chain of the antibody of the invention. In other words, the light chain and the heavy chain of the antibody may be encoded by the same nucleic acid molecule (e.g., in a bicistronic manner). Alternatively, the light chain and the heavy chain of the antibody may be encoded by distinct nucleic acid molecules.

Due to the redundancy of the genetic code, the present invention also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promoter, a heterologous enhancer, a heterologous UTR (e.g., for optimal translation/expression), a heterologous Poly-A-tail, and the like.

A nucleic acid molecule is a molecule comprising nucleic acid components. The term nucleic acid molecule usually refers to DNA or RNA molecules. It may be used synonymous with the term “polynucleotide,” i.e., the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers that are covalently linked to each other by phosphodiester-bonds of a sugar/phosphate-backbone. The term “nucleic acid molecule” also encompasses modified nucleic acid molecules, such as base-modified, sugar-modified or backbone-modified, etc. DNA or RNA molecules.

In general, the nucleic acid molecule may be manipulated to insert, delete, or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent,” i.e., not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody of the invention can be randomly or directionally mutated to introduce different properties in the encoded amino acids. Such changes can be the result of an iterative process wherein initial changes are retained, and new changes at other nucleotide positions are introduced. Further, changes achieved in independent steps may be combined.

In some embodiments, the polynucleotide encoding the antibody, or an antigen binding fragment thereof, (or the (complete) nucleic acid molecule) may be codon-optimized. The skilled artisan is aware of various tools for codon optimization, such as those described in: Ju Xin Chin, et al., Bioinformatics, Volume 30, Issue 15, 1 Aug. 2014, Pages 2210-2212; or in: Grote A, et al. Nucleic Acids Res. 2005 Jul. 1; 33(Web Server issue):W526-31; or, for example, Genscript's OptimumGene™ algorithm (as described in US 2011/0081708 A1).

For example, the nucleic acid of the invention may comprise a nucleic acid sequence as set forth in any one of SEQ ID NOs 20-25 or a sequence variant thereof having 70% or more (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) sequence identity.

The present invention also provides a combination of a first and a second nucleic acid molecule, wherein the first nucleic acid molecule comprises a polynucleotide encoding the heavy chain of the antibody of the present invention and the second nucleic acid molecule comprises a polynucleotide encoding the corresponding light chain of the same antibody. The above description regarding the (general) features of the nucleic acid molecule of the invention applies accordingly to the first and second nucleic acid molecules of the combination. For example, one or both of the polynucleotides encoding the heavy and/or light chain(s) of the antibody may be codon-optimized.

### C. Vector

Further included within the scope of the invention are vectors, for example, expression vectors, comprising a nucleic acid molecule according to the present invention. Usually, a vector comprises a nucleic acid molecule as described above.

The present invention also provides a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a (recombinant) nucleic acid molecule, which does not occur in nature. Accordingly, the vector may comprise heterologous elements (i.e., sequence elements of different origins in nature). For example, the vector may comprise a multi cloning site, a heterologous promoter, a heterologous enhancer, a heterologous selection marker (to identify cells comprising said vector in comparison to cells not comprising said vector) and the like. A vector in the context of the present invention is suitable for incorporating or harboring a desired nucleic acid sequence. Such vectors may be storage vectors, expression vectors, cloning vectors, transfer vectors, etc. A storage vector is a vector which allows the convenient storage of a nucleic acid molecule. Thus, the vector may comprise a sequence corresponding, e.g., to a (heavy and/or light chain of a) desired antibody according to the present invention. An expression vector may be used for production of expression products such as RNA, e.g., mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

### D. Cells

In a further aspect, the present invention also provides cells expressing the antibody according to the present invention; and/or comprising the vector according to the present invention.

Examples of such cells include but are not limited to, eukaryotic cells, e.g., yeast cells, animal cells or plant cells or prokaryotic cells, including E. coli. In some embodiments, the cells are mammalian cells, such as a mammalian cell line. Examples include human cells, CHO cells, HEK293T cells, PER.C6 cells, NS0 cells, human liver cells, myeloma cells or hybridoma cells.

The cell may be transfected with a vector according to the present invention, for example, with an expression vector. The term “transfection” refers to the introduction of nucleic acid molecules, such as DNA or RNA (e.g., mRNA) molecules, into cells, e.g., into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g., based on cationic lipids and/or liposomes, calcium phosphate precipitation, nanoparticle-based transfection, virus-based transfection, or transfection based on cationic polymers, such as DEAE-dextran or polyethylenimine, etc. In some embodiments, the introduction is non-viral.

Moreover, the cells of the present invention may be transfected stably or transiently with the vector according to the present invention, e.g., for expressing the antibody according to the present invention. In some embodiments, the cells are stably transfected with the vector according to the present invention encoding the antibody according to the present invention. In other embodiments, the cells are transiently transfected with the vector according to the present invention encoding the antibody according to the present invention.

Accordingly, the present invention also provides a recombinant host cell, which heterologously expresses the antibody of the invention or the antigen binding fragment thereof. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state or abolish a PTM on the antibody that is present in the antibody's native state. Such an additional or removed PTM may result in a functional difference (e.g., reduced immunogenicity). Accordingly, the antibody of the invention, or the antigen binding fragment thereof, may have a post-translational modification, which is distinct from the naturally produced antibody (e.g., an antibody of an immune response in a human).

### E. Production of Antibodies

Antibodies according to the present invention can be made by any method known in the art. For example, the general methodology for making monoclonal antibodies using hybridoma technology is well known (Kohler, G. and Milstein, C. 1975; Kozbar et al. 1983). In some embodiments, the alternative EBV immortalization method described in WO2004/076677 is used.

In some embodiments, the method, as described in WO 2004/076677, which is incorporated herein by reference, is used. In this method, B cells producing the antibody of the invention are transformed with EBV and a polyclonal B cell activator. Additional stimulants of cellular growth and differentiation may optionally be added during the transformation step to further enhance the efficiency. These stimulants may be cytokines such as IL-2 and IL-15. In one aspect, IL-2 is added during the immortalization step to further improve the efficiency of immortalization, but its use is not essential. The immortalized B cells produced using these methods can then be cultured using methods known in the art and antibodies isolated therefrom.

Another exemplified method is described in WO 2010/046775. In this method, plasma cells are cultured in limited numbers, or as single plasma cells in microwell culture plates. Antibodies can be isolated from plasma cell cultures. Further, from the plasma cell cultures, RNA can be extracted and PCR can be performed using methods known in the art. The VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Standard techniques of molecular biology may be used to prepare DNA sequences encoding the antibodies of the present invention. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of nucleic acid sequences encoding the antibody molecules of the present invention. Eukaryotic, e.g., mammalian, host cell expression systems may be used for production of antibody molecules, such as complete antibody molecules. Suitable mammalian host cells include, but are not limited to, CHO, HEK293T, PER.C6, NS0, myeloma or hybridoma cells. In other embodiments, prokaryotic cells, including, but not limited to, E. coli, may be used for the expression of nucleic acid sequences encoding the antibody molecules of the present invention.

The present invention also provides a process for the production of an antibody molecule according to the present invention comprising culturing a (heterologous) host cell comprising a vector encoding a nucleic acid of the present invention under conditions suitable for expression of protein from DNA encoding the antibody molecule of the present invention, and isolating the antibody molecule.

For production of the antibody comprising both heavy and light chains, a cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Antibodies according to the invention may be produced by (i) expressing a nucleic acid sequence according to the invention in a host cell, e.g., by use of a vector according to the present invention, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by an immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned, and expressed in HEK293T cells or other known host cells using methods known in the art.

The immortalized B cell clones or the transfected host-cells of the invention can be used in various ways, e.g., as a source of monoclonal antibodies, as a source of nucleic acid (DNA or mRNA) encoding a monoclonal antibody of interest, for research, etc.

The invention also provides a composition comprising immortalized B memory cells or transfected host cells that produce antibodies according to the present invention.

The immortalized B cell clone or the cultured plasma cells of the invention may also be used as a source of nucleic acid for the cloning of antibody genes for subsequent recombinant expression. Expression from recombinant sources may be more common for pharmaceutical purposes than expression from B cells or hybridomas, e.g., for reasons of stability, reproducibility, culture ease, etc.

Thus the invention also provides a method for preparing a recombinant cell, comprising the steps of: (i) obtaining one or more nucleic acids (e.g., heavy and/or light chain mRNAs) from the B cell clone or the cultured plasma cells that encodes the antibody of interest; (ii) inserting the nucleic acid into an expression vector and (iii) transfecting the vector into a (heterologous) host cell in order to permit expression of the antibody of interest in that host cell.

Similarly, the invention also provides a method for preparing a recombinant cell, comprising the steps of: (i) sequencing nucleic acid(s) from the B cell clone or the cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest, wherein the nucleic acids are nucleic acids that were derived from an immortalized B cell clone or a cultured plasma cell of the invention. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Methods for obtaining and sequencing immunoglobulin genes from B cells or plasma cells are well known in the art (e.g., see Chapter 4 of Kuby Immunology, 4th edition, 2000).

The transfected host cell may be a eukaryotic cell, including yeast and animal cells, particularly mammalian cells (e.g., CHO cells, NS0 cells, human cells such as PER.C6 or HKB-11 cells, myeloma cells, or a human liver cell), as well as plant cells. In some embodiments, the transfected host cell may a prokaryotic cell, including E. coli. In some embodiments, the transfected host cell is a mammalian cell, such as a human cell. In some embodiments, expression hosts can glycosylate the antibody of the invention, particularly with carbohydrate structures that are not themselves immunogenic in humans. In some embodiments, the transfected host cell may be able to grow in serum-free media. In further embodiments, the transfected host cell may be able to grow in culture without the presence of animal-derived products. The transfected host cell may also be cultured to give a cell line.

The invention also provides a method for preparing one or more nucleic acid molecules (e.g., heavy and light chain genes) that encode an antibody of interest, comprising the steps of: (i) preparing an immortalized B cell clone or culturing plasma cells according to the invention; to (ii) obtaining from the B cell clone or the cultured plasma cells nucleic acid that encodes the antibody of interest. Further, the invention provides a method for obtaining a nucleic acid sequence that encodes an antibody of interest, comprising the steps of: (i) preparing an immortalized B cell clone or culturing plasma cells according to the invention; (ii) sequencing nucleic acid from the B cell clone or the cultured plasma cells that encodes the antibody of interest.

The invention further provides a method of preparing nucleic acid molecule(s) that encode an antibody of interest, comprising the step of obtaining the nucleic acid that was obtained from a transformed B cell clone or cultured plasma cells of the invention. Thus the procedures for first obtaining the B cell clone or the cultured plasma cell, and then obtaining nucleic acid(s) from the B cell clone or the cultured plasma cells can be performed at different times by different people in different places (e.g., in different countries).

The invention also comprises a method for preparing an antibody (e.g., for pharmaceutical use) according to the present invention, comprising the steps of: (i) obtaining and/or sequencing one or more nucleic acids (e.g., heavy and light chain genes) from the selected B cell clone or the cultured plasma cells expressing the antibody of interest; (ii) inserting the nucleic acid(s) into or using the nucleic acid(s) sequence(s) to prepare an expression vector; (iii) transfecting a host cell that can express the antibody of interest; (iv) culturing or sub-culturing the transfected host cells under conditions where the antibody of interest is expressed; and, optionally, (v) purifying the antibody of interest.

The invention also provides a method of preparing the antibody of interest comprising the steps of: culturing or sub-culturing a transfected host cell population, e.g., a stably transfected host cell population, under conditions where the antibody of interest is expressed and, optionally, purifying the antibody of interest, wherein said transfected host cell population has been prepared by (i) providing nucleic acid(s) encoding a selected antibody of interest that is produced by a B cell clone or cultured plasma cells prepared as described above, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest. Thus the procedures for first preparing the recombinant host cell and then culturing it to express antibodies can be performed at very different times by different people in different places (e.g., in different countries).

### F. Pharmaceutical Composition

The present invention also provides a pharmaceutical composition comprising one or more of:

(i) the antibody according to the present invention;

(ii) the nucleic acid encoding the antibody according to the present invention;

(iii) the vector comprising the nucleic acid according to the present invention; and/or

(iv) the cell expressing the antibody according to the present invention or comprising the vector according to the present invention;

and, optionally, a pharmaceutically acceptable diluent or carrier.

In other words, the present invention also provides a pharmaceutical composition comprising the antibody according to the present invention, the nucleic acid according to the present invention, the vector according to the present invention and/or the cell according to the present invention.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers, and inactive virus particles. In some embodiments, the pharmaceutically acceptable carrier, diluent and/or excipient in the pharmaceutical composition according to the present invention is not an active component in respect to influenza A virus infection.

Pharmaceutically acceptable salts can be used, for example, mineral acid salts, such as hydrochlorides, hydrobromides, phosphates, and sulfates, or salts of organic acids, such as acetates, propionates, malonates, and benzoates.

Pharmaceutically acceptable carriers in a pharmaceutical composition may additionally contain liquids such as water, saline, glycerol, and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin®, for reconstitution with sterile water containing a preservative). The composition may be prepared for topical administration, e.g., as an ointment, cream or powder. The composition may be prepared for oral administration, e.g., as a tablet or capsule, as a spray, or as a syrup (optionally flavored). The composition may be prepared for pulmonary administration, e.g., as an inhaler, using a fine powder or a spray. The composition may be prepared as a suppository or pessary. The composition may be prepared for nasal, aural or ocular administration, e.g., as drops. The composition may be in kit form, designed such that a combined composition is reconstituted just prior to administration to a subject. For example, a lyophilized antibody may be provided in kit form with sterile water or a sterile buffer.

In some embodiments, the (only) active ingredient in the composition is the antibody according to the present invention. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH between 5.5 and 8.5, in some embodiments, this may be between 6 and 8, for example about 7. The pH may be maintained by the use of a buffer. The composition may be sterile and/or pyrogen-free. The composition may be isotonic with respect to humans. In some embodiments, pharmaceutical compositions of the invention are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle, and it may contain formulatory agents, such as suspending, preservative, stabilizing and/or dispersing agents. Alternatively, the antibody may be in dry form, for reconstitution before use with an appropriate sterile liquid.

A vehicle is typically understood to be a material that is suitable for storing, transporting, and/or administering a compound, such as a pharmaceutically active compound, in particular, the antibodies according to the present invention. For example, the vehicle may be a physiologically acceptable liquid, which is suitable for storing, transporting, and/or administering a pharmaceutically active compound, in particular, the antibodies according to the present invention. Once formulated, the compositions of the invention can be administered directly to the subject. In some embodiments, the compositions are adapted for administration to mammalian, e.g., human subjects.

The pharmaceutical compositions of this invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the invention. Optionally, the pharmaceutical composition may be prepared for oral administration, e.g., as tablets, capsules, and the like, for topical administration, or as injectable, e.g., as liquid solutions or suspensions. In some embodiments, the pharmaceutical composition is an injectable. Solid forms suitable for solution in, or suspension in, liquid vehicles prior to injection are also encompassed, for example, the pharmaceutical composition may be in lyophilized form.

For injection, e.g., intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is an antibody, a peptide, a nucleic acid molecule, or another pharmaceutically useful compound according to the present invention that is to be given to an individual, administration is usually in a “prophylactically effective amount” or a “therapeutically effective amount” (as the case may be), this being sufficient to show benefit to the individual. The actual amount administered, and rate and time-course of administration, will depend on the nature and severity of what is being treated. For injection, the pharmaceutical composition according to the present invention may be provided, for example, in a pre-filled syringe.

The inventive pharmaceutical composition as defined above may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e., the inventive transporter cargo conjugate molecule, as defined above, is combined with emulsifying and suspending agents. If desired, certain sweetening, flavoring or coloring agents may also be added.

The inventive pharmaceutical composition may also be administered topically, especially when the target of treatment includes areas or organs readily accessible by topical application, e.g., including accessible epithelial tissue. Suitable topical formulations are readily prepared for each of these areas or organs. For topical applications, the inventive pharmaceutical composition may be formulated in a suitable ointment, containing the inventive pharmaceutical composition, particularly its components, as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax, and water. Alternatively, the inventive pharmaceutical composition can be formulated in a suitable lotion or cream. In the context of the present invention, suitable carriers include, but are not limited to, mineral oil, sorbitan monostearate, Polysorbate 60, cetyl esters wax, Cetearyl alcohol, 2-octyldodecanol, benzyl alcohol, and water.

Dosage treatment may be a single dose schedule or a multiple-dose schedule. In particular, the pharmaceutical composition may be provided as a single-dose product. In some embodiments, the amount of the antibody in the pharmaceutical composition—in particular, if provided as a single-dose product—does not exceed 200 mg, for example, it does not exceed 100 mg or 50 mg.

For example, the pharmaceutical composition according to the present invention may be administered daily, e.g., once or several times per day, e.g., once, twice, three times or four times per day, for 1, 2, 3, 4, 5, 6, 7, 8, 9, 10, 11, 12, 13, 14, 15, 16, 17, 18, 19, 20 or 21 or more days, e.g., daily for 1, 2, 3, 4, 5, 6 months. In some embodiments, the pharmaceutical composition according to the present invention may be administered weekly, e.g., once or twice per week, for 1, 2, 3, 4, 5, 6, 7, 8, 9, 10, 11, 12, 13, 14, 15, 16, 17, 18, 19, 20 or 21 or more weeks, e.g., weekly for 1, 2, 3, 4, 5, 6, 7, 8, 9, 10, 11, or 12 months or weekly for 2, 3, 4, or 5 years. Moreover, the pharmaceutical composition according to the present invention may be administered monthly, e.g., once per month or every second month for 1, 2, 3, 4, or 5 or more years. Administration may also continue for the lifetime. In some embodiments, one single administration only is also envisaged, in particular with respect to certain indications, e.g., for prophylaxis of influenza A virus infection. For example, a single administration (single dose) is administered, and further doses may be administered at one or more later time points, when the titer of the antibody is insufficient or assumed to be insufficient for protection.

For a single dose, e.g., a daily, weekly or monthly dose, the amount of the antibody in the pharmaceutical composition according to the present invention, may not exceed 1 g or 500 mg. In some embodiments, for a single dose, the amount of the antibody in the pharmaceutical composition according to the present invention, may not exceed 200 mg, or 100 mg. For example, for a single dose, the amount of the antibody in the pharmaceutical composition according to the present invention, may not exceed 50 mg.

Pharmaceutical compositions typically include an “effective” amount of one or more antibodies of the invention, i.e., an amount that is sufficient to treat, ameliorate, attenuate, reduce or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. For purposes of the present invention, an effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody of the present invention (e.g., amount of the antibody in the pharmaceutical composition) in relation to the bodyweight (e.g., in kg) of the individual to which it is administered.

Moreover, the pharmaceutical composition according to the present invention may also comprise an additional active component, which may be a further antibody or a component, which is not an antibody. For example, the pharmaceutical composition may comprise one or more antivirals (which are not antibodies). Moreover, the pharmaceutical composition may also comprise one or more antibodies (which are not according to the invention), for example, an antibody against other influenza virus antigens (other than hemagglutinin) or an antibody against another influenza virus (e.g., against an influenza B virus or against an influenza C virus). Accordingly, the pharmaceutical composition according to the present invention may comprise one or more of the additional active components.

The antibody according to the present invention can be present either in the same pharmaceutical composition as the additional active component or, alternatively, the antibody according to the present invention is comprised by a first pharmaceutical composition, and the additional active component is comprised by a second pharmaceutical composition different from the first pharmaceutical composition. Accordingly, if more than one additional active component is envisaged, each additional active component and the antibody according to the present invention may be comprised in a different pharmaceutical composition. Such different pharmaceutical compositions may be administered either combined/simultaneously or at separate times or at separate locations (e.g., separate parts of the body).

The antibody according to the present invention and the additional active component may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In some embodiments, a composition of the invention may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In the composition of the invention, the antibodies may be in purified form.

The present invention also provides a method of preparing a pharmaceutical composition comprising the steps of: (i) preparing an antibody of the invention; and (ii) admixing the purified antibody with one or more pharmaceutically acceptable carriers.

In other embodiments, a method of preparing a pharmaceutical composition comprises the step of: admixing an antibody with one or more pharmaceutically-acceptable carriers, wherein the antibody is a monoclonal antibody that was obtained from a transformed B cell or a cultured plasma cell of the invention.

As an alternative to delivering antibodies or B cells for therapeutic purposes, it is possible to deliver nucleic acid (typically DNA) that encodes the monoclonal antibody of interest derived from the B cell or the cultured plasma cells to a subject, such that the nucleic acid can be expressed in the subject in situ to provide a desired therapeutic effect. Suitable gene therapy and nucleic acid delivery vectors are known in the art.

Pharmaceutical compositions may include an antimicrobial, particularly if packaged in a multiple-dose format. They may comprise detergent, e.g., a Tween (polysorbate), such as Tween 80. Detergents are generally present at low levels, e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose), e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The compositions of the invention may also comprise one or more immunoregulatory agents. In some embodiments, one or more of the immunoregulatory agents include(s) an adjuvant.

### G. Medical Treatments and Uses

In a further aspect, the present invention provides the use of the antibody according to the present invention, the nucleic acid according to the present invention, the vector according to the present invention, the cell according to the present invention or the pharmaceutical composition according to the present invention in (i) prophylaxis and/or treatment of infection with influenza A virus; or in (ii) diagnosis of infection with influenza A virus. Accordingly, the present invention also provides a method of reducing influenza A virus infection, or lowering the risk of influenza A virus infection, comprising: administering to a subject in need thereof, a therapeutically effective amount of the antibody according to the present invention, the nucleic acid according to the present invention, the vector according to the present invention, the cell according to the present invention or the pharmaceutical composition according to the present invention. Moreover, the present invention also provides the use of the antibody according to the present invention, the nucleic acid according to the present invention, the vector according to the present invention, the cell according to the present invention or the pharmaceutical composition according to the present invention in the manufacture of a medicament for prophylaxis, treatment or attenuation of influenza A virus infection.

Methods of diagnosis may include contacting an antibody with a sample. Such samples may be isolated from a subject, for example, an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood, such as plasma or serum. The methods of diagnosis may also include the detection of an antigen/antibody complex, in particular following the contacting of an antibody with a sample. Such a detection step is typically performed at the bench, i.e., without any contact with the human or animal body. Examples of detection methods are well-known to the person skilled in the art and include, e.g., ELISA (enzyme-linked immunosorbent assay).

Prophylaxis of infection with influenza A virus refers in particular to prophylactic settings, wherein the subject was not diagnosed with infection with influenza A virus (either no diagnosis was performed or diagnosis results were negative) and/or the subject does not show symptoms of infection with influenza A virus. Prophylaxis of infection with influenza A virus is particularly to useful in subjects at greater risk of severe disease or complications when infected, such as pregnant women, children (such as children under 59 months), the elderly, individuals with chronic medical conditions (such as chronic cardiac, pulmonary, renal, metabolic, neurodevelopmental, liver or hematologic diseases) and individuals with immunosuppressive conditions (such as HIV/AIDS, receiving chemotherapy or steroids, or malignancy). Moreover, prophylaxis of infection with influenza A virus is also particularly useful in subjects at greater risk acquiring influenza A virus infection, e.g., due to increased exposure, for example, subjects working or staying in public areas, in particular, health care workers.

In therapeutic settings, in contrast, the subject is typically infected with influenza A virus, diagnosed with influenza A virus infection and/or showing symptoms of influenza A virus infection. Of note, the terms “treatment” and “therapy”/“therapeutic” of influenza A virus infection include (complete) cure as well as attenuation/reduction of influenza A virus infection and/or related symptoms.

Accordingly, the antibody according to the present invention, the nucleic acid according to the present invention, the vector according to the present invention, the cell according to the present invention or the pharmaceutical composition according to the present invention may be used for treatment of influenza A virus infection in subjects diagnosed with influenza A virus infection or in subjects showing symptoms of influenza A virus infection.

The antibody according to the present invention, the nucleic acid according to the present invention, the vector according to the present invention, the cell according to the present invention or the pharmaceutical composition according to the present invention may also be used for prophylaxis and/or treatment of influenza A virus infection in asymptomatic subjects. Those subjects may be diagnosed or not diagnosed with influenza A virus infection.

In some embodiments, the antibody according to the present invention, the nucleic acid according to the present invention, the vector according to the present invention, the cell according to the present invention or the pharmaceutical composition according to the present invention may be administered prophylactically or therapeutically. For example, the antibody according to the present invention, the nucleic acid according to the present invention, the vector according to the present invention, the cell according to the present invention or the pharmaceutical composition according to the present invention is used for prophylaxis and/or treatment of influenza A virus infection, wherein the antibody, the nucleic acid, the vector, the cell, or the pharmaceutical composition is administered up to three months before (a possible) influenza A virus infection or up to one month before (a possible) influenza A virus infection, such as up to two weeks before (a possible) influenza A virus infection or up to one week before (a possible) influenza A virus infection. For example, the antibody according to the present invention, the nucleic acid according to the present invention, the vector according to the present invention, the cell according to the present invention or the pharmaceutical composition according to the present invention is used for prophylaxis and/or treatment of influenza A virus infection, wherein the antibody, the nucleic acid, the vector, the cell, or the pharmaceutical composition is administered up to one day before (a possible) influenza A virus infection. Such a treatment schedule refers, in particular, to a prophylactic setting.

Moreover, the antibody according to the present invention, the nucleic acid according to the present invention, the vector according to the present invention, the cell according to the present invention or the pharmaceutical composition according to the present invention may be used for prophylaxis and/or treatment of influenza A virus infection, wherein the antibody, the nucleic acid, the vector, the cell, or the pharmaceutical composition is administered up to three months before the first symptoms of influenza A infection occur or up to one month before the first symptoms of influenza A infection occur, such as up to two weeks the first symptoms of influenza A infection occur or up to one week before the first symptoms of influenza A infection occur. For example, the antibody according to the present invention, the nucleic acid according to the present invention, the vector according to the present invention, the cell according to the present invention or the pharmaceutical composition according to the present invention is used for prophylaxis and/or treatment of influenza A virus infection, wherein the antibody, the nucleic acid, the vector, the cell, or the pharmaceutical composition is administered up to three days or two days before the first symptoms of influenza A infection occur.

In general after the first administration of the antibody according to the present invention, the nucleic acid according to the present invention, the vector according to the present invention, the cell according to the present invention or the pharmaceutical composition according to the present invention, one or more subsequent administrations may follow, for example, a single dose per day or per every second day for 1, 2, 3, 4, 5, 6, 7, 8, 9, 10, 11, 12, 13, 1, 15, 16, 17, 18, 19, 20, or 21 days. After the first administration of the antibody according to the present invention, the nucleic acid according to the present invention, the vector according to the present invention, the cell according to the present invention or the pharmaceutical composition according to the present invention, one or more subsequent administrations may follow, for example, a single dose once or twice per week for 1, 2, 3, 4, 5, 6, 7, 8, 9, 10, 11, 12, 13, 1, 15, 16, 17, 18, 19, 20, or 21 weeks. After the first administration of the antibody according to the present invention, the nucleic acid according to the present invention, the vector according to the present invention, the cell according to the present invention or the pharmaceutical composition according to the present invention, one or more subsequent administrations may follow, for example, a single dose every 2 or 4 weeks for 1, 2, 3, 4, 5, 6, 7, 8, 9, 10, 11, 12, 13, 1, 15, 16, 17, 18, 19, 20, or 21 weeks. After the first administration of the antibody according to the present invention, the nucleic acid according to the present invention, the vector according to the present invention, the cell according to the present invention or the pharmaceutical composition according to the present invention, one or more subsequent administrations may follow, for example, a single dose every two or four months for 1, 2, 3, 4, 5, 6, 7, 8, 9, 10, 11, 12, 13, 1, 15, 16, 17, 18, 19, 20, or 21 months. After the first administration of the antibody according to the present invention, the nucleic acid according to the present invention, the vector according to the present invention, the cell according to the present invention or the pharmaceutical composition according to the present invention, one or more subsequent administrations may follow, for example, a single dose once or twice per year for 1, 2, 3, 4, 5, 6, 7, 8, 9, or 10 years.

In some embodiments, the antibody according to the present invention, the nucleic acid according to the present invention, the vector according to the present invention, the cell according to the present invention or the pharmaceutical composition according to the present invention is administered at a (single) dose of 0.005 to 100 mg/kg bodyweight or 0.0075 to 50 mg/kg bodyweight, such as at a (single) dose of 0.01 to 10 mg/kg bodyweight or at a (single) dose of 0.05 to 5 mg/kg bodyweight. For example, the antibody according to the present invention, the nucleic acid according to the present invention, the vector according to the present invention, the cell according to the present invention or the pharmaceutical composition according to the present invention is administered at a (single) dose of 0.1 to 1 mg/kg bodyweight.

The antibody according to the present invention, the nucleic acid according to the present invention, the vector according to the present invention, the cell according to the present invention or the pharmaceutical composition according to the present invention may be administered by any number of routes such as oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes.

In some embodiments, the antibody according to the present invention, the nucleic acid according to the present invention, the vector according to the present invention, the cell according to the present invention or the pharmaceutical composition according to the present invention is administered prophylactically, i.e., before a diagnosis of influenza A infection.

In some embodiments, the antibody of the invention may be administered to subjects at immediate risk of influenza A infection. An immediate risk of influenza A infection typically occurs during an influenza A epidemic. Influenza A viruses are known to circulate and cause seasonal epidemics of disease (WHO, Influenza (Seasonal) Fact sheet, Nov. 6, 2018). In temperate climates, seasonal epidemics occur mainly during winter, while in tropical regions, influenza may occur throughout the year, causing outbreaks more irregularly. For example, in the northern hemisphere, the risk of an influenza A epidemic is high during November, December, January, February, and March, while in the southern hemisphere the risk of an influenza A epidemic is high during May, June, July, August, and September.

H. Combination therapy

The administration of the antibody according to the present invention, the nucleic acid according to the present invention, the vector according to the present invention, the cell according to the present invention or the pharmaceutical composition according to the present invention in the methods and uses according to the invention can be carried out alone or in combination with a co-agent (also referred to as “additional active component” herein), which may be useful for preventing and/or treating influenza infection.

The invention encompasses the administration of the antibody according to the present invention, the nucleic acid according to the present invention, the vector according to the present invention, the cell according to the present invention or the pharmaceutical composition according to the present invention, wherein it is administered to a subject prior to, simultaneously with or after a co-agent or another therapeutic regimen useful for treating and/or preventing influenza. Said antibody, nucleic acid, vector, cell or pharmaceutical composition, that is administered in combination with said co-agent can be administered in the same or different composition(s) and by the same or different route(s) of administration. As used herein, expressions like “combination therapy,” “combined administration,” “administered in combination” and the like are intended to refer to a combined action of the drugs (which are to be administered “in combination”). To this end, the combined drugs are usually present at a site of action at the same time and/or at an overlapping time window. It may also be possible that the effects triggered by one of the drugs are still ongoing (even if the drug itself may not be present anymore) while the other drug is administered, such that effects of both drugs can interact. However, a drug which was administered long before another drug (e.g., more than one, two, three or more months or a year), such that it is not present anymore (or its effects are not ongoing) when the other drug is administered, is typically not considered to be administered “in combination.” For example, influenza medications administered in distinct influenza seasons are usually not administered “in combination.”

Said other therapeutic regimens or co-agents may be, for example, an antiviral. An antiviral (or “antiviral agent” or “antiviral drug”) refers to a class of medication used specifically for treating viral infections. Like antibiotics for bacteria, antivirals may be broad-spectrum antivirals useful against various viruses or specific antivirals that are used for specific viruses. Unlike most antibiotics, antiviral drugs do usually not destroy their target pathogen; instead, they typically inhibit their development.

Thus, in another aspect of the present invention the antibody, or an antigen binding fragment thereof, according to the present invention, the nucleic acid according to the present invention, the vector according to the present invention, the cell according to the present invention or the pharmaceutical composition according to the present invention is administered in combination with (prior to, simultaneously or after) an antiviral for the (medical) uses as described herein.

In general, an antiviral may be a broad-spectrum antiviral (which is useful against influenza viruses and other viruses) or an influenza virus-specific antiviral. In some embodiments, the antiviral is not an antibody. For example, the antiviral may be a small molecule drug. Examples of small molecule antivirals useful in prophylaxis and/or treatment of influenza are described in Wu X, et al. Theranostics. 2017; 7(4):826-845. As described in Wu et al., 2017, the skilled artisan is familiar with various antivirals useful in prophylaxis and/or treatment of influenza. Further antivirals useful in influenza are described in Davidson S. Front Immunol. 2018; 9:1946; and in Koszalka P, et al. Influenza Other Respir Viruses. 2017; 11(3):240-246.

Antivirals useful in prophylaxis and/or treatment of influenza include (i) agents targeting functional proteins of the influenza virus itself and (ii) agents targeting host cells, e.g., the epithelium.

Host cell targeting agents include the thiazolide class of broad-spectrum antivirals, sialidase fusion proteins, type III interferons, Bcl-2 (B cell lymphoma 2) inhibitors, protease inhibitors, V-ATPase inhibitors, and antioxidants. Examples of the thiazolide class of broad-spectrum antivirals include nitazoxanide (NTZ), which is rapidly deacetylated in the blood to the active metabolic form tizoxanide (TIZ), and second-generation thiazolide compounds, which are structurally related to NTZ, such as RM5061. Fludase (DAS181) is an example of sialidase fusion proteins. Type III IFNs include, for example, IFNλ. Non-limiting examples of Bcl-2 inhibitors include ABT-737, ABT-263, ABT-199, WEHI-539, and A-1331852 (Davidson S. Front Immunol. 2018; 9:1946). Examples of protease inhibitors include nafamostat, Leupeptin, epsilon-aminocaproic acid, Camostat, and Aprotinin. V-ATPase inhibitors include NorakinR, ParkopanR, AntiparkinR, and AkinetonR. An example of an antioxidant is alpha-tocopherol.

In some embodiments, the antiviral is an agent targeting a functional protein of the influenza virus itself. For example, the antiviral may target a functional protein of the influenza virus, which is not hemagglutinin. In general, antivirals targeting a functional protein of the influenza virus include entry inhibitors, hemagglutinin inhibitors, neuraminidase inhibitors, influenza polymerase inhibitors (RNA-dependent RNA polymerase (RdRp) inhibitors), nucleocapsid protein inhibitors, M2 ion channel inhibitors, and arbidol hydrochloride. Non-limiting examples of entry inhibitors include triterpenoids derivatives, such as glycyrrhizic acid (glycyrrhizin) and glycyrrhetinic acid; saponins; uralsaponins M-Y (such as uralsaponins M); dextran sulfate (DS); silymarin; curcumin; and lysosomotropic agents, such as Concanamycin A, Bafilomycin A1, and Chloroquine. Non-limiting examples of hemagglutinin inhibitors include BMY-27709; stachyflin; natural products, such as Gossypol, Rutin, Quercetin, Xylopine, and Theaflavins; trivalent glycopeptide mimetics, such as compound 1 described in Wu X, Wu X, Sun Q, et al. Progress of small molecular inhibitors in the development of anti-influenza virus agents. Theranostics. 2017; 7(4):826-845; podocarpic acid derivatives, such as compound 2 described in Wu X, et al. Theranostics. 2017; 7(4):826-845; natural product pentacyclic triterpenoids, such as compound 3 described in Wu X, et al. Theranostics. 2017; 7(4):826-845; and prenylated indole diketopiperazine alkaloids, such as Neoechinulin B. Non-limiting examples of nucleocapsid protein inhibitors include nucleozin, Cycloheximide, Naproxen, and Ingavirin. Non-limiting examples of M2 ion channel inhibitors include the approved M2 inhibitors Amantadine and Rimantadine and derivatives thereof; as well as non-adamantane derivatives, such as Spermine, Spermidine, Spiropiperidine, and pinanamine derivatives.

In some embodiments, the antiviral is selected from neuraminidase (NA) inhibitors and influenza polymerase inhibitors (RNA-dependent RNA polymerase (RdRp) inhibitors). Non-limiting examples of neuraminidase (NA) inhibitors include zanamivir; oseltamivir; peramivir; laninamivir; derivatives thereof such as compounds 4-10 described in Wu X, et al. Theranostics. 2017; 7(4):826-845, and dimeric zanamivir conjugates (e.g., as described in Wu X, et al. Theranostics. 2017; 7(4):826-845); benzoic acid derivatives (e.g., as described in Wu X, et al. Theranostics. 2017; 7(4):826-845; such as compounds 11-14); pyrrolidine derivatives (e.g., as described in Wu X, et al. Theranostics. 2017; 7(4):826-845; such as compounds 15-18); ginkgetin-sialic acid conjugates; flavanones and flavonoids isoscutellarein and its derivatives (e.g., as described in Wu X, et al. Theranostics. 2017; 7(4):826-845); AV5080; and N-substituted oseltamivir analogues (e.g., as described in Wu X, et al. Theranostics. 2017; 7(4):826-845). Non-limiting examples of influenza polymerase inhibitors (RNA-dependent RNA polymerase (RdRp)) inhibitors include RdRp disrupting compounds, such as those described in Wu X, et al. Theranostics. 2017; 7(4):826-845; PB2 cap-binding inhibitors, such as JNJ63623872 (VX-787); cap-dependent endonuclease inhibitors, such as baloxavir marboxil (S-033188); PA endonuclease inhibitors, such as AL-794, EGCG and its aliphatic analogues, N-hydroxamic acids and N-hydroxyimides, flutamide and its aromatic analogues, tetramic acid derivatives, L-742,001, ANA-0, polyphenolic catechins, phenethyl-phenylphthalimide analogues, macrocyclic bisbibenzyls, pyrimidines, fullerenes, hydroxyquinolines, hydroxypyridinones, hydroxypyridazinones, trihydroxy-phenyl-bearing compounds, 2-hydroxy-benzamides, hydroxy-pyrimidinones, β-diketo acid and its bioisosteric compounds, thiosemicarbazones, bisdihydroxyindole-carboxamides, and pyrido-piperazinediones (Endo-1); and nucleoside and nucleobase analogue inhibitors, such as ribavirin, favipiravir (T-705), 2′-Deoxy-2′-fluoroguanosine (2′-FdG), 2′-substituted carba-nucleoside analogues, 6-methyl-7-substituted-7-deaza purine nucleoside analogues, and 2′-deoxy-2′-fluorocytidine (2′-FdC). For example, the antiviral may be zanamivir, oseltamivir or baloxavir.

Thus, the pharmaceutical composition according to the present invention may comprise one or more of the additional active components. The antibody according to the present invention can be present in the same pharmaceutical composition as the additional active component (co-agent). Alternatively, the antibody according to the present invention and the additional active component (co-agent) are comprised in distinct pharmaceutical compositions (e.g., not in the same composition). Accordingly, if more than one additional active component (co-agent) is envisaged, each additional active component (co-agent) and the antibody, or the antigen binding fragment, according to the present invention may be comprised by a different pharmaceutical composition. Such different pharmaceutical compositions may be administered either combined/simultaneously or at separate times and/or by separate routes of administration.

The antibody according to the present invention and the additional active component (co-agent) may provide an additive or a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

Accordingly, the present invention also provides a combination of (i) the antibody of the invention as described herein, and (ii) an antiviral agent as described above.

### Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is comprised of a heavy chain variable region (abbreviated herein as VII) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

The term “antigen-binding fragment or portion” of an antibody (or simply “antibody fragment or portion”), as used herein, refers to one or more fragments of an antibody that retain the ability to specifically bind to an antigen (e.g., a Spike or S protein of SARS-CoV-2 virus). It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fab′ fragment, which is essentially a Fab with part of the hinge region (see, FUNDAMENTAL IMMUNOLOGY (Paul ed., 3rd ed. 1993)); (iv) a Fd fragment consisting of the VH and CHI domains; (v) a Fv fragment consisting of the VL and VH domains of a single arm of an antibody, (vi) a dAb fragment (Ward et al., (1989) Nature 341:544-546), which consists of a VH domain; (vii) an isolated CDR; to and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). In some embodiments, human monoclonal antibodies are prepared by using improved EBV-B cell immortalization as described in Traggiai E, et al. (2004). Nat Med. 10(8):871-5.

As used herein, the term “variable region” (variable region of a light chain (V_{L}), variable region of a heavy chain (V_{H})) denotes each of the pair of light and heavy chains which is involved directly in binding the antibody to the antigen.

Antibodies of the invention can be of any isotype (e.g., IgA, IgG, IgM, i.e., an α, γ or μ heavy chain). For example, the antibody is of the IgG type. Within the IgG isotype, antibodies may be IgG1, IgG2, IgG3 or IgG4 subclass, for example, IgG1. Antibodies of the invention may have a κ or a λ light chain. In some embodiments, the antibody is of IgG1 type and has a κ light chain.

Antibodies according to the present invention may be provided in purified form. Typically, the antibody will be present in a composition that is substantially free of other polypeptides, e.g., where less than 90% (by weight), usually less than 60% and more usually less than 50% of the composition is made up of other polypeptides.

Antibodies according to the present invention may be immunogenic in human and/or in nonhuman (or heterologous) hosts, e.g., in mice. For example, the antibodies may have an idiotope that is immunogenic in nonhuman hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

The term “antigen binding portion” or “antigen binding fragment” of an antibody, as used herein, refers to one or more fragments of an antibody that retain the ability to specifically bind to an antigen (e.g., HA of influenza A virus). Examples of binding fragments encompassed within the term “antigen binding portion/fragment” of an antibody include (i) a Fab fragment—a monovalent fragment consisting of the V_{L}, V_{H}, C_{L} and CH1 domains; (ii) a F(ab′)2 fragment—a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, and (v) a dAb fragment (Ward et al. (1989) Nature 341:544-546) consisting of a V_{H} domain. An isolated complementarity determining region (CDR), or a combination of two or more isolated CDRs joined by a synthetic linker, may comprise an antigen binding domain of an antibody that is able to bind antigen.

The term “monoclonal antibody,” as used herein, refers to an antibody that displays a single binding specificity and affinity for a particular epitope or a composition of antibodies in which all antibodies display a single binding specificity and affinity for a particular epitope. Typically such monoclonal antibodies will be derived from a single cell or nucleic acid encoding the antibody, and will be propagated without intentionally introducing any sequence alterations. Accordingly, the term “human monoclonal antibody” refers to a monoclonal antibody that has variable and optional constant regions derived from human germline immunoglobulin sequences. In one embodiment, human monoclonal antibodies are produced by a hybridoma, for example, obtained by fusing a B cell obtained from a transgenic or transchromosomal non-human animal (e.g., a transgenic mouse having a genome comprising a human heavy chain transgene and a light chain transgene), to an immortalized cell.

Single chain antibody constructs are also included in the invention. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird et al. (1988) Science 242:423-426; and Huston et al. (1988) Proc. Natl. Acad. Sci. (USA) 85:5879-5883). Such single chain antibodies are also intended to be encompassed within the term “antigen binding portion/fragment” of an antibody. These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen binding portions/fragments can be produced by recombinant DNA techniques or by enzymatic or chemical cleavage of intact immunoglobulins.

A “bispecific” or “bifunctional antibody” is an artificial hybrid antibody having two different heavy/light chain pairs, giving rise to two antigen binding sites with specificity for different antigens. Bispecific antibodies can be produced by a variety of methods including fusion of hybridomas or linking of Fab′ fragments. See, e.g., Songsivilai & Lachmann (1990) Clin. Exp. Immunol. 79:315-321; Kostelny et al. (1992) J. Immunol. 148, 1547-1553.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. Human antibodies of the present invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies are used synonymously.

The term “human monoclonal antibody” refers to antibodies displaying a single binding specificity, which have variable regions in which both the framework and CDR regions are derived from human germline immunoglobulin sequences. In one embodiment, the human monoclonal antibodies can be produced by a hybridoma that includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

A “humanized” antibody refers to an antibody in which some, most or all of the amino acids outside the CDR domains of a non-human antibody, e.g., a mouse antibody, are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications can be made within the human framework sequences.

The term “chimeric antibody” is intended to refer to antibodies in which the variable region sequences are derived from one species, and the constant region sequences are derived from another species, such as an antibody in which the variable region sequences are derived from a mouse antibody, and the constant region sequences are derived from a human antibody. The term can also refer to an antibody in which its variable region sequence or CDR(s) is derived from one source (e.g., an IgA1 antibody) and the constant region sequence or Fc is derived from a different source (e.g., a different antibody, such as an IgG, IgA2, IgD, IgE or IgM antibody).

The phrases “an antibody recognizing an antigen” and “an antibody specific for an antigen” are used interchangeably herein with the term “an antibody that binds specifically to an antigen.”

As used herein, a “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used alone, or in combination, as prophylactic or therapeutic agents upon appropriate formulation, in association with active vaccination, as a diagnostic tool, or as a production tool as described herein.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide or protein. For example, a peptide or polypeptide fragment can have is at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length. Preferably the peptide fragment can elicit an immune response when used to inoculate an animal. A peptide fragment may be used to elicit an immune response by inoculating an animal with a peptide fragment in combination with an adjuvant, a peptide fragment that is coupled to an adjuvant, or a peptide fragment that is coupled to arsanilic acid, sulfanilic acid, an acetyl group, or a picryl group. A peptide fragment can include a non-amide bond and can be a peptidomimetic.

As used herein, the term “mutation” relates to a change in the nucleic acid sequence and/or in the amino acid sequence in comparison to a reference sequence, e.g., a corresponding genomic sequence. A mutation, e.g., in comparison to a genomic sequence, may be, for example, a (naturally occurring) somatic mutation, a spontaneous mutation, an induced mutation, e.g., induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g., in a nucleic acid sequence or in an amino acid sequence. A mutation includes substitution, deletion, and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved, e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

A “nucleic acid” or “polynucleotide” refers to a DNA molecule (for example, but not limited to, a cDNA or genomic DNA) or an RNA molecule (for example, but not limited to, an mRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

Throughout this specification and the claims which follow, unless the context requires otherwise, the term “comprise,” and variations such as “comprises” and “comprising,” will be understood to imply the inclusion of a stated member, integer or step but not the exclusion of any other non-stated member, integer or step. The term “consist of” is a particular embodiment of the term “comprise,” wherein any other non-stated member, integer or step is excluded. In the context of the present invention, the term “comprise” encompasses the term “consist of” The term “comprising” thus encompasses “including” as well as “consisting,” e.g., a composition “comprising” X may consist exclusively of X or may include something additional, e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human). The subject may be a human or a non-human. In this context, a “normal,” “control,” or “reference” subject, patient or population is/are one(s) that exhibit(s) no detectable disease or disorder, respectively.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

An “effective amount” refers to the amount of an active compound/agent that is required to confer a therapeutic effect on a treated subject. Effective doses will vary, as recognized by those skilled in the art, depending on the types of conditions treated, route of administration, excipient usage, and the possibility of co-usage with other therapeutic treatment. A therapeutically effective amount of a combination to treat a neoplastic condition is an amount that will cause, for example, a reduction in tumor size, a reduction in the number of tumor foci, or slow the growth of a tumor, as compared to untreated animals.

As used herein, “administering” refers to the physical introduction of a composition comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “specifically binding” and similar reference does not encompass non-specific sticking.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols, and reagents described herein as these may vary. It is also to be understood that the terminology used herein is to describe particular embodiments only and is not intended to limit the scope of the present invention which will be limited only by the appended claims. Unless defined otherwise, all technical and scientific terms used herein have the same meanings as commonly understood by one of ordinary skill in the art.

### EXAMPLES

In the following, particular examples illustrating various embodiments and aspects of the invention are presented. However, the present invention shall not be limited in scope by the specific embodiments described herein. The following preparations and examples are given to enable those skilled in the art to more clearly understand to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Materials and Methods

Viruses, Cell Lines, and Mouse Strains

The A/Puerto Rico/8/34 (PR8) and A/Netherlands/602/09 (Neth09) H1N1 viruses were grown in 10-day old specific pathogen-free embryonated chicken eggs (CHARLES RIVER LABORATORIES), as described previously⁶. MDCK cells (ATCC) were maintained at 37° C., 5% CO₂ in DMEM supplemented with 10% FBS, 50 U/ml penicillin and 50 μg/ml streptomycin (THERMOFISHER). Expi293 cells were maintained at 37° C., 8% CO₂ in Expi293 expression medium (THERMOFISHER) supplemented with 10 U/ml penicillin and 10 μg/ml streptomycin. All in vivo experiments were performed in compliance with federal laws and institutional guidelines and have been approved by the Rockefeller University Institutional Animal Care and Use Committee. Mice were bred and maintained at the Comparative Bioscience Center at the Rockefeller University. FcγR humanized mice (FcγRαₙᵤₗₗ, hFcγRI⁺, FcγRIIa^{R131+}, FcγRIIb⁺, FcγRIIIa^{F158+}, and FcγRIIIb⁺) were generated in the C57Bl/6 background and extensively characterized in previous studies¹⁰. FcRn humanized mice (B6.Cg-Fcgrttm1Dcr Tg(FCGRT)32Dcr/DcrJ) were purchased from The Jackson Laboratory and are deficient in mouse FcRn and express human FcRn as transgene^{19,20}. FcγR/FcRn humanized mice were generated by crossing the FcγR humanized strain to the FcRn humanized mice.

Cloning, Expression, and Purification of Recombinant IgG Antibodies

For the generation of Fc domain variants of human IgG1 Fc domain variants, site-directed mutagenesis using specific primers was performed based on the QuikChange site-directed mutagenesis Kit II (AGILENT TECHNOLOGIES), as previously described⁴. Recombinant antibodies were generated by transient transfection of Expi293 cells with heavy and light chain expression plasmids, using previously described protocols²¹. Prior to transfection, plasmid sequences were validated by direct sequencing (GENEWIZ). Recombinant IgG antibodies were purified from cell-free supernatants by affinity purification using Protein G or Protein A sepharose beads (GE Healthcare). Purified proteins were dialyzed in PBS, filter-sterilized (0.22 μm), and purity was assessed by SDS-PAGE followed by SafeStain blue staining (THERMOFISHER). All antibody preparations were >90% pure and endotoxin levels were <0.005 EU/mg, as determined by the Limulus Amebocyte Lysate (LAL) assay. For the generation of afucosylated Fc domain variants, CHO cells were transfected with heavy chain and light chain expression plasmids in the presence of 100 μM 2-fluorofucose peracetate²². To confirm the absence of fucose, glycans were released with PNGase F, labeled with Waters RapiFluor-MS, cleaned up with a HILIC microElution plate, injected onto a Waters Glycan BEH Amide column, using a Thermo Vanquish UHPLC with FLD detection. Chromatograms were integrated and the relative contribution of each glycan calculated as a percentage. Peaks were identified by mass spec using a Thermo Q Exactive Plus mass spectrometer and through comparison to the NIST mAb standard.

Anti-HA and NA ELISA

Recombinant HA (Influenza A H1N1 (A/California/04/2009 or A/Puerto Rico/8/34) or NA (A/California/04/2009) (Sinobiological) (3 μg/ml) were immobilized into high-binding 96-well microtiter plates (Nunc) and following overnight incubation at 4° C., plates were blocked with PBS+2% (w/v) BSA+0.05% (v/v) Tween20 for 2 h. After blocking, plates were incubated for 1 h with serially-diluted IgG antibodies (1:3 consecutive dilutions in PBS starting at 1 μg/ml), followed by HRP-conjugated goat anti-human IgG (1 h; 1:5000; JACKSON IMMUNORESEARCH). Plates were developed using the TMB (3,3′,5,5′-Tetramethylbenzidine) two-component peroxidase substrate kit (KPL) and reactions stopped with the addition of 1 M phosphoric acid. Absorbance at 450 nm was immediately recorded using a SpectraMax Plus spectrophotometer (MOLECULAR DEVICES), and background absorbance from negative control samples was subtracted.

Microneutralization Assay

The neutralizing activity of anti-HA and NA mAb Fc variants was evaluated in microneutralization assays, using previously described protocols²³. Virus input was titrated to maximize the signal-to-noise ratio. Fc domain variants of mAbs (starting concentration at 100 μg/ml followed by 1:3 serial dilutions) and viruses (1.8×10³ pfu/ml for A/Puerto Rico/8/34 and 3.2×10⁴ pfu/ml for A/Netherlands/602/09) were prepared in DMEM supplemented with 50 U/ml penicillin, 50 μg/ml streptomycin, 25 mM HEPES and 1 μg/ml TPCK-treated trypsin (Sigma). Virus-mAb mixture was pre-incubated for 1 h at 37° C. and added to a monolayer of MDCK cells (70-80% confluent in 96-well plates). Following incubation at 37° C. for 1 h to allow for virus adsorption, the cell monolayer was washed three times with PBS and re-incubated for 18-20 h at 37° C. with medium (DMEM supplemented with 50 U/ml penicillin, 50 μg/ml streptomycin, 25 mM HEPES and 1 μg/ml TPCK-treated trypsin) containing mAbs (at equivalent concentrations as during the virus co-incubation). Cells were fixed with 80% (v/v) acetone, blocked with 5% (w/v) non-fat milk diluted in PBS for 30 min at room temperature, and quenched with 3% (v/v) hydrogen peroxide (in PBS) by incubating for a further 20 min at room temperature. Cells were stained with biotinylated anti-NP antibody (EMD Millipore; 1:2000), followed by HRP-conjugated streptavidin (Jackson Immunoresearch). Plates were developed using the TMB (3,3′,5,5′-Tetramethylbenzidine) two-component peroxidase substrate kit (KPL) and reactions stopped with the addition of 1 M phosphoric acid. Absorbance at 450 nm was immediately recorded using a SpectraMax Plus spectrophotometer (Molecular Devices), and background absorbance from negative control samples was subtracted.

Hemagglutination Inhibition (HAI) Assay

HAI activity was evaluated using previously described protocols²⁴. Briefly, Fc domain variants of mAbs (starting concentration at 100 μg/ml followed by 1:3 serial dilutions) and viruses (A/Puerto Rico/8/34 or A/Netherlands/602/09; 10⁷ pfu/ml) were incubated in V-bottom 96 microtiter plates for 30 min at room temperature. Turkey RBCs (0.75% (v/v); Rockland) were added to the mAb:virus mixture, mixed gently and incubated for 30 min at room temperature. Plates were scored for the number of wells exhibiting HAI activity.

Quantification of Serum IgG Levels

Blood from mice was collected into gel microvette tubes, serum was fractionated by centrifugation (10,000 g, 5 min) and stored at −20° C. IgG levels in serum samples were determined by ELISA following previously published protocols²¹. Briefly, high-binding 96-well microtiter plates (Nunc) were coated overnight at 4° C. with Neutravidin (2 μg/ml in PBS). All sequential steps were performed at room temperature. Plates were blocked for 1 h with PBS/2% (w/v) BSA and incubated with biotinylated goat anti-human IgG antibodies for 1 h (5 μg/ml; Jackson Immunoresearch). Serum samples were serially diluted and incubated for 1 h, followed by incubation with horseradish peroxidase-conjugated anti-human IgG (1:5000). Plates were developed using the TMB (3,3′,5,5′-Tetramethylbenzidine) two-component peroxidase substrate kit (KPL) and reactions stopped with the addition of 1 M phosphoric acid. Absorbance at 450 nm was immediately recorded using a SpectraMax Plus spectrophotometer (Molecular Devices) and background absorbance from negative control samples was subtracted.

Mouse Influenza Infection Models

Mice (females; 6-12 weeks old) were anesthetized with a ketamine (75 mg/kg)/xylazine (15 mg/kg) mixture (administered i.p.) and viruses (diluted in PBS) were administered intranasally (5 mLD₅₀) in 30 μl. Following infection, mice were monitored daily, and their weights were recorded for 14 d. Death was determined by a 20% body weight loss threshold that was authorized by the Rockefeller University Institutional Animal Care and Use Committee. For mAb-mediated prophylaxis, mAbs were administered i.p. or i.v. 4 h prior to virus challenge (except for experiments with FcRn/FcγR humanized mice, where mAbs were administered 2 days prior to infection), whereas for mAb-mediated therapy, mAbs were administered on day 3 post-infection. Antibody dose was calculated as mg/kg.

In Vivo CD8⁺ T-Cell Depletion

CD8⁺ cells were depleted in mice by administration of anti-CD8 mAbs. To establish the efficiency of mAb-mediated CD8⁺ T-cell depletion, FcγR humanized mice were injected i.v. with 150 μg anti-mouse CD8a mAb (clone 2.43; rat IgG2b; Bioxcell) or isotype control (clone LTF-2; rat IgG2b; Bioxcell). The abundance of CD8⁺ T cells in peripheral blood was determined at various time points following mAb administration by flow cytometry. Baseline CD8⁺ T-cell frequencies were determined in blood samples obtained prior to mAb administration. For the flow cytometry analysis, fluorescently conjugated mAbs targeting the β subunit of mouse CD8 (clone Ly-3; Biolegend) were used to avoid competition with the depleting mAb, which targets the a subunit of CD8. CD8⁺ T cell depletion of influenza-infected mice was performed using the aforementioned conditions, and depleting mAbs or isotype were administered i.v. on day 3 post-infection.

Processing of Lung Tissues and Flow Cytometry Analysis

Mice were euthanized and lungs were perfused by injection of PBS (containing 10 U/ml heparin) into the right cardiac ventricle. Lungs were excised and homogenized using the gentleMACS dissociator (Mouse lung dissociation kit (MILTENYI)), according to the manufacturer's recommendations. Following RBC lysis (RBC lysis buffer; BIOLEGEND), single cell suspensions were labelled with the LIVE/DEAD Fixable Near-IR (THERMOFISHER) and resuspended in PBS containing 0.5% (w/v) BSA and 5 mM EDTA. Cells were labelled with mixtures of fluorescently labelled antibodies including: (i) for the evaluation of FcγR expression in innate effector leukocytes: anti-CD11c-eFluor506, anti-human FcγRI (clone 10.1)-BrilliantViolet 605, anti-SiglecF-SuperBright 645, anti-Ly6G-BrilliantViolet 711, anti-CD11b-BrilliantViolet 785, anti-human FcγRIIa (clone IV.3)-FITC, anti-Ly6C-PerCP/Cy5.5, anti-human FcγRIIIa/b (clone 3G8)-PE, anti-CD103-PE/eFluor610, anti-NK1.1-PE/Cy7, and anti-human FcγRIIb (clone 2B6)-Dylight 680; (ii) for the evaluation of FcγR expression and activation status of DCs: anti-CD11c-eFluor506, anti-human FcγRI (clone 10.1)-BrilliantViolet 605, anti-SiglecF-SuperBright 645, anti-CD80-BrilliantViolet 711, anti-CD11b-BrilliantViolet 785, anti-human FcγRIIa (clone IV.3)-FITC, anti-GR-1-PerCP/Cy5.5, anti-human FcγRIIIa/b (clone 3G8)-PE, anti-CD103-PE/eFluor610, anti-CD86-PE/Cy7, anti-human FcγRIIb (clone 2B6)-Dylight 680, and anti-MHCII-AlexaFluor 700; (iii) for the evaluation of CD8 depletion: anti-CD3e-BrilliantViolet 510, anti-CD19-BrilliantViolet 605, anti-CD8b-BrilliantViolet 711, anti-CD11b-PE, anti-NK1.1-PE/Cy7, anti-CD4-FITC, anti-GR-1-PerCP/Cy5.5, anti-NKp46-eFluor660, and anti-B220-APC/eFluor780; (iv) for the characterization of DC populations following mAb treatment: anti-CD103-FITC, anti-Ly6C-PerCP/Cy5.5, anti-NK1.1-AlexaFluor 647, anti-CD45-AlexaFluor 700, anti-CD11c-eFluor 506, anti-CD86-BrilliantViolet 605, anti-SiglecF—SuperBright 645, anti-Ly6G-BrilliantViolet 711, anti-CD11b-BrilliantViolet 785, anti-CD40-PE, anti-MHCII-PE/eFluor 610, and anti-CD80, PE/Cy7; (v) for the characterization of T-cell populations following mAb treatment: anti-CD4-AlexaFluor 488, anti-CD3e-PerCP/Cy5.5, anti-NK1.1-AlexaFluor 647, anti-CD45-AlexaFluor 700, anti-CD44-BrilliantViolet 421, anti-CD62L-BrilliantViolet 510, anti-CD25-BrilliantViolet 605, anti-CD27-BrilliantViolet 650, anti-CD8-BrilliantViolet 711, anti-CD11a-PE, anti-CCR7-PE/eFluor 610, and anti-CD69-PE/Cy7. Cell counts were determined using CountBright absolute counting beads (ThermoFisher). Samples were collected on an Attune NxT flow cytometer (THERMOFISHER) and analyzed using FlowJo (v10.6) software. For cluster analysis, DCs (defined as Live/Lin⁺/CD45⁺/CD11e/MHCII⁺) and T cells (defined as Live/CD45⁺/NK1.1⁻/CD3⁺) from individual mice were downsampled (3000 (DCs) or 6000 (T cells) events/mouse; 12000 (DCs) or 24000 (T cells)/treatment condition) and concatenated. Cells were clustered and visualized using UMAP reduction and populations were identified by KNN density estimation (X-shift)²⁵.

Statistical Analysis

Results from multiple experiments are presented as mean±standard error of the mean (SEM). One- or two-way ANOVA was used to test for differences in the mean values of quantitative variables, and where statistically significant effects were found, post-hoc analysis using Bonferroni multiple comparison test was performed. Statistical differences between survival rates were analyzed by comparing Kaplan-Meier curves using the log-rank (Mantel-Cox) test. Data were analyzed with Graphpad Prism software (GRAPHPAD), and P values of <0.05 were considered to be statistically significant.

### Example 1: Effector Functions are Crucial for Antibody-Mediated Protection Against Influenza Infection

One of the crucial mechanisms of action of a therapeutic antibody is the targeted elimination of viruses and virus-infected cells through recruitment of the immune system. This is typically achieved by interaction of the antibody's Fc domain with Fcγ receptors (FcγRs; FcgammaRs; FcgRs) and/or the complement component C1 q. In view thereof, the role of these effector functions in antibody-mediated protection against influenza was investigated.

An antibody comprising (i) the CDR sequences as set forth in SEQ ID NOs: 1-6 (or 1-4, 11, and 6, respectively) and (ii) the two mutations M428L and N434S in the heavy chain constant region, was designed and produced. More specifically, the antibody comprises (i) the heavy chain variable region (VH) sequence as set forth in SEQ ID NO: 7 and the light chain variable region (VL) sequence as set forth in SEQ ID NO: 8; and (ii) the two mutations M428L and N434S in the heavy chain constant region. Even more specifically, the antibody comprises a heavy chain having an amino acid sequence as set forth in SEQ ID NO: 13 and a light chain having an amino acid sequence as set forth in SEQ ID NO: 10. This antibody is referred to herein as “Flu1_MLNS”. In particular, the constant regions of antibody “Flu1_MLNS” do not comprise any other mutations (other than M428L and N434S).

For comparison, antibody “Flu1_MLNS+GRLR” was designed and produced which differs from antibody “Flu1_MLNS” only in that it also comprises, in its heavy chain constant region, the two mutations G236R and L328R, which abrogate binding to Fcγ receptors (FcγRs, FcgRs) and complement protein C1q (Horton, H. M. et al. (2010). Blood 116, 3004-3012; Bournazos S. et al. Cell. 2014; 158(6):1243-1253) in addition to the two mutations M428L and N434S. Accordingly, this antibody has a heavy chain having an amino acid sequence as set forth in SEQ ID NO: 16 and a light chain having an amino acid sequence as set forth in SEQ ID NO: 10.

The antibodies were tested in an influenza infection model (lethal challenge) in transgenic C57BL/6 mice lacking all classes of mouse FcγRs and expressing all human FcγRs (FcγR humanized mice, as described in Smith, P. et al. (Smith, P., et al. Proc Natl Acad Sci USA 109, 6181-6186, doi:10.1073/pnas.1203954109 (2012)). Mouse model recapitulating human Fcγ receptor structural and functional diversity. Proc Natl Acad Sci USA. 2012; 109(16):6181-6). FcγR humanized female mice aged 6-10 weeks were allocated to eight distinct groups (n=4-6 per group) for testing different doses (0.5 mg/kg, 1 mg/kg, 2 mg/kg or 4 mg/kg) for each antibody (Flu1_MLNS or Flu1_MLNS+GRLR). The antibody was administered intraperitoneally 4 h prior to intranasal infection with a lethal dose (5 mLD₅₀) of influenza virus A/Puerto Rico/8/34 H1N1 (PR8). Animals were monitored for disease severity and symptoms for a period of 14 days, and bodyweight was recorded daily. Mice with >20% loss in bodyweight were humanely euthanized by CO₂ asphyxiation using methods and procedures consistent with the recommendations of the American Veterinary Medical Association (AVMA) Guidelines for the Euthanasia of Animals. Mice were also humanely euthanized if they showed signs of respiratory distress, including hunched appearance, ruffled fur, labored breathing, and lethargy. Blood samples were obtained on day 4 after infection (retro-orbitally or via the submandibular vein), and the levels of Flu1 antibody in the serum of treated mice were determined by ELISA using anti-human IgG detection antibodies. Log-rank (Mantel-Cox) test was used to compare endpoint survival between experimental groups and one-way ANOVA (Tukey posthoc test) to test for differences in bodyweight, serum antibody levels, and other quantitative variables.

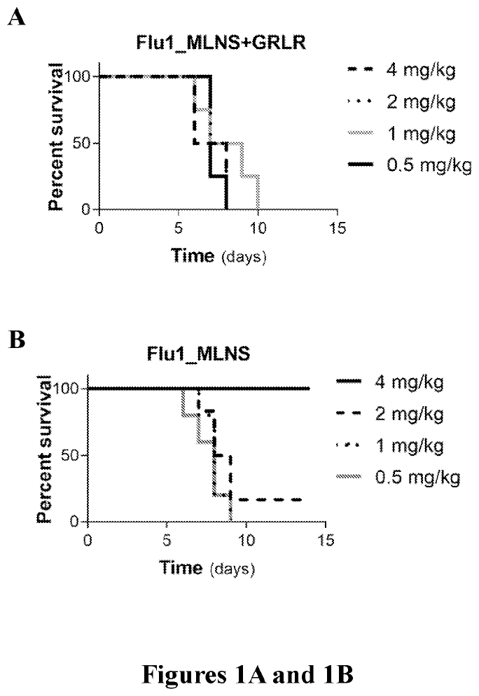

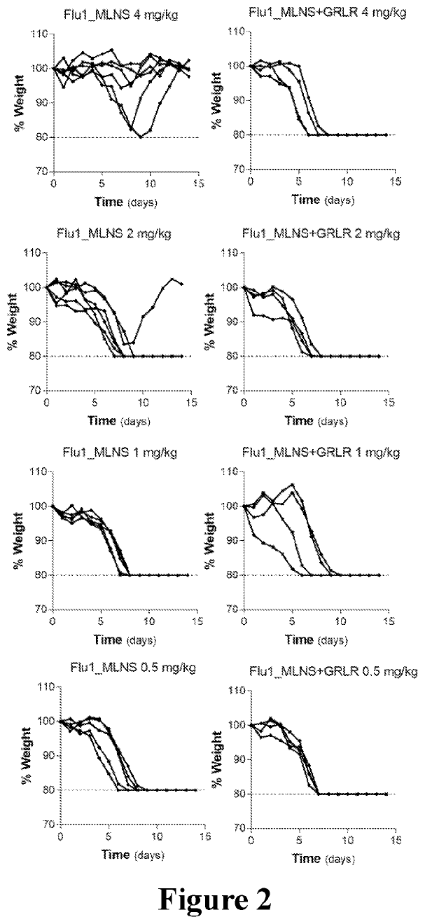

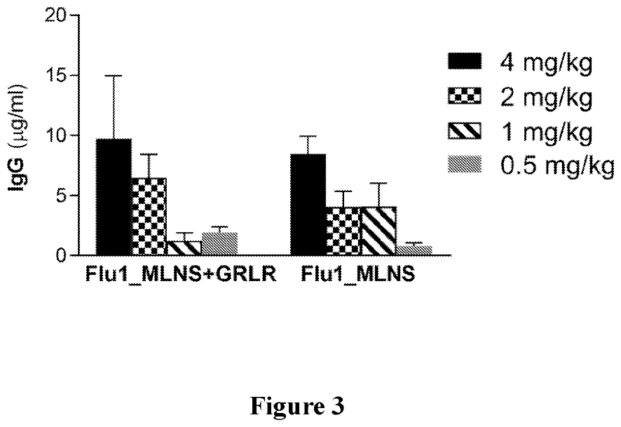

The results are shown in FIGS. 1-3. FIG. 1 shows the survival rates of mice receiving different doses of antibodies Flu1_MLNS or Flu1_MLNS+GRLR prior to lethal challenge with PR8 influenza virus. All mice receiving Flu1_MLNS+GRLR (in which binding to FcγRs and C1q is abrogated) died, independent from antibody dosage. In contrast, one mouse receiving 2 mg/kg Flu1_MLNS and all mice receiving 4 mg/kg Flu1_MLNS survived, thereby showing significant effects (p=0.0018 for 4 mg/kg Flu1_MLNS vs. 4 mg/kg Flu1_MLNS+GRLR, Log-rank (Mantel-Cox) test). FIG. 2 shows the course of the bodyweight after infection for each mouse in each group (as indicated in the figure). FIG. 3 shows the levels of Flu1_MLNS and Flu1_MLNS+GRLR in the serum of treated mice, as measured on day 4 post infection. For each antibody dose, the respective Flu1_MLNS and Flu1_MLNS+GRLR group showed comparable IgG levels.

In summary, the data show that the antibody-mediated its protective effects via effector functions, while loss of binding to FcγRs and Clq (by the GRLR mutation) resulted in loss of protection.

### Example 2: Antibodies of the Invention Show Increased Protection Against Influenza Infection

I. Design of Antibodies of the Invention and Dose-Response Experiments

In view of the crucial role of antibody's effector functions, an antibody of the invention was designed and produced, which comprises, in its heavy chain constant region, the three mutations G236A, A330L, and I332E. More specifically, antibody “Flu1_MLNS+GAALIE” comprises (i) the CDR sequences as set forth in SEQ ID NOs 1-6 (or 1-4, 11, and 6, respectively) and (ii) the five mutations G236A, A330L, I332E, M428L, and N434S in the heavy chain constant regions. Even more specifically, the antibody comprises (i) the heavy chain variable region (VH) sequence as set forth in SEQ ID NO: 7 and the light chain variable region (VL) sequence as set forth in SEQ ID NO: 8; and (ii) the five mutations G236A, A330L, I332E, M428L, and N434S in the heavy chain constant regions. Still, more specifically, the antibody comprises a heavy chain having an amino acid sequence as set forth in SEQ ID NO: 14 and a light chain having an amino acid sequence as set forth in SEQ ID NO: 10. This antibody is referred to herein as “Flu1_MLNS+GAALIE.” Accordingly, Flu1_MLNS+GAALIE differs from Flu1_MLNS (cf. Example 1) only in the three mutations G236A, A330L, and I332E.

Different doses of antibody Flu1_MLNS+GAALIE (0.5, 1, 2, 4, 8, or 16 mg/kg) were tested in different groups of transgenic C57BL/6 mice lacking all classes of mouse FcγRs, but expressing human FcγRs (FcγR humanized mice; as described in Example 1). As control, a further group of mice received phosphate-buffered saline (PBS). The experiments were performed essentially as described in Example 1. Briefly, the antibody (or PBS) was administered intraperitoneally 4 h prior to infection with a lethal dose (5 mLD₅₀) of influenza virus A/Puerto Rico/8/34 H1N1 (PR8). Animals were monitored, and bodyweight was recorded daily. Blood samples were obtained on day 4 after infection, and the Flu1 antibody levels in the serum of treated animals were determined by ELISA as described in Example 1.

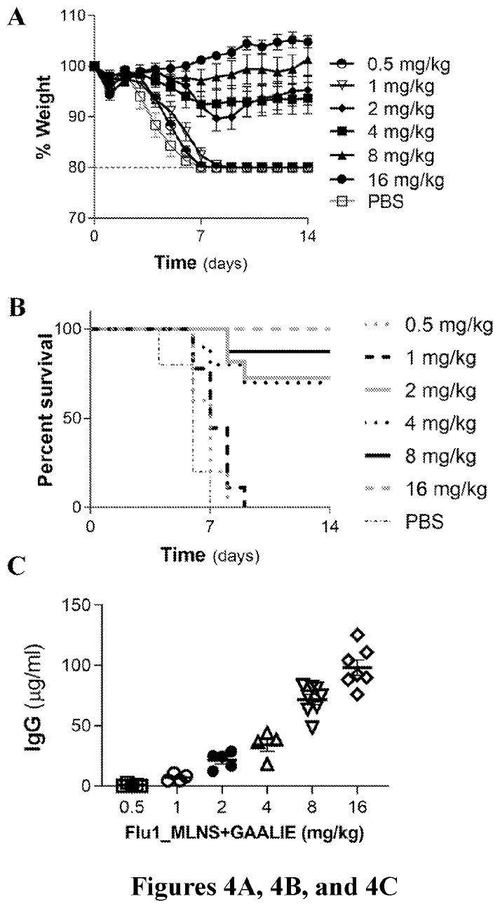

The results are shown in FIG. 4. FIG. 4 shows that increasing doses of the antibody resulted in dose-dependent protection against infection, as evidenced by milder reduction in bodyweight (FIG. 4A) and improved survival rates (FIG. 4B) after a lethal influenza challenge. Serum levels of Flu1_MLNS+GAAL1E were determined on day 4 following antibody treatment and correlated with the dose of the administered antibody (FIG. 4C). The data show that a dose of 2 mg/kg was the “limiting” (minimum effective) dose of antibody “Flu1_MLNS+GAALIE” to protect FcγR humanized mice against lethal influenza challenge.

II. Antibodies of the Invention Provide Superior Protection Against Influenza Infection

Next, antibody Flu1_MLNS+GAALIE was compared to antibodies Flu1_MLNS and Flu1_MLNS+GRLR (cf. Example 1) in lethal challenge experiments.

As a positive control, an afucosylated version of antibody Flu1_MLNS was produced (“Flu1_MLNSafuc” or “Flu1_MLNSafucosylated”). Afucosylated antibodies are engineered so that the oligosaccharides in the Fc region of the antibody do not comprise any fucose sugar units. Afucosylation is known to increase antibody-dependent cellular cytotoxicity (ADCC). To obtain Flu1_MLNSafuc, CHO cells were transfected with heavy chain and light chain expression plasmids in the presence of 100 μM 2-fluorofucose peracetate (Okeley, N. M., et al. (2013). PNAS, 110(14), 5404-5409). To check the level of fucose, the following method was performed. Glycans were released with PNGase F, labeled with Waters RapiFluor-MS, cleaned up with a HILIC microElution plate, injected onto a Waters Glycan BEH Amide column, using a Thermo Vanquish UHPLC with FLD detection. Chromatograms were integrated, and the relative contribution of each glycan was calculated as a percentage. Peaks were identified by mass spec using a Thermo Q Exactive Plus mass spectrometer and through comparison to the NIST mAb standard.

Different groups of transgenic C57BL/6 mice lacking all mouse FcγRs, but expressing human FcγRs (FcγR humanized mice; as described in Example 1) received 2 mg/kg of Flu1_MLNS, Flu1_MLNS+GRLR, Flu1_MLNS+GAALIE or Flu1_MLNSafuc. As control, a further group of mice received phosphate-buffered saline (PBS). The experiments were performed essentially as described in Example 1. Briefly, the antibody (or PBS) was administered intraperitoneally 4 h prior to infection with a lethal dose (5 mLD₅₀) of influenza virus A/Puerto Rico/8/34 H1N1 (PR8). Animals were monitored, and bodyweight was recorded. Blood samples were obtained on day 4 after infection, and Flu1 antibody levels were determined as described in Example 1.

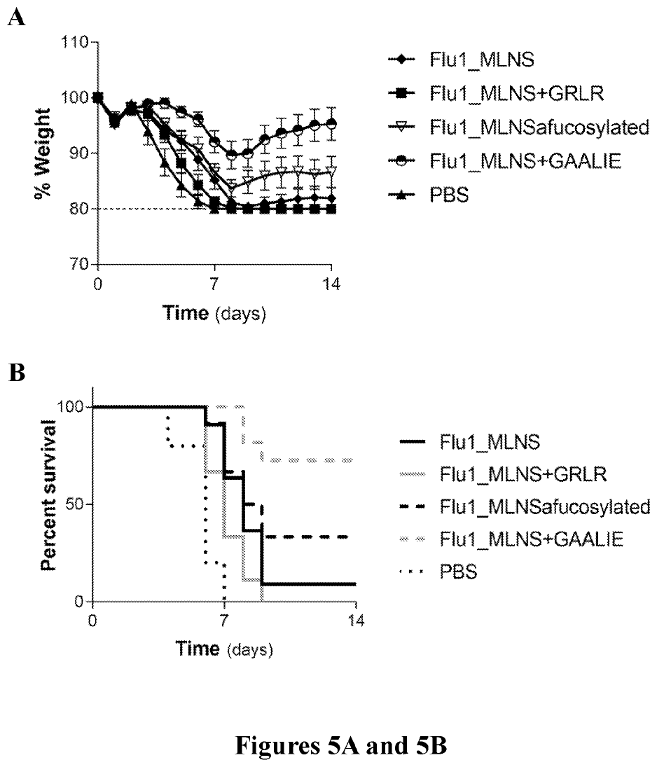

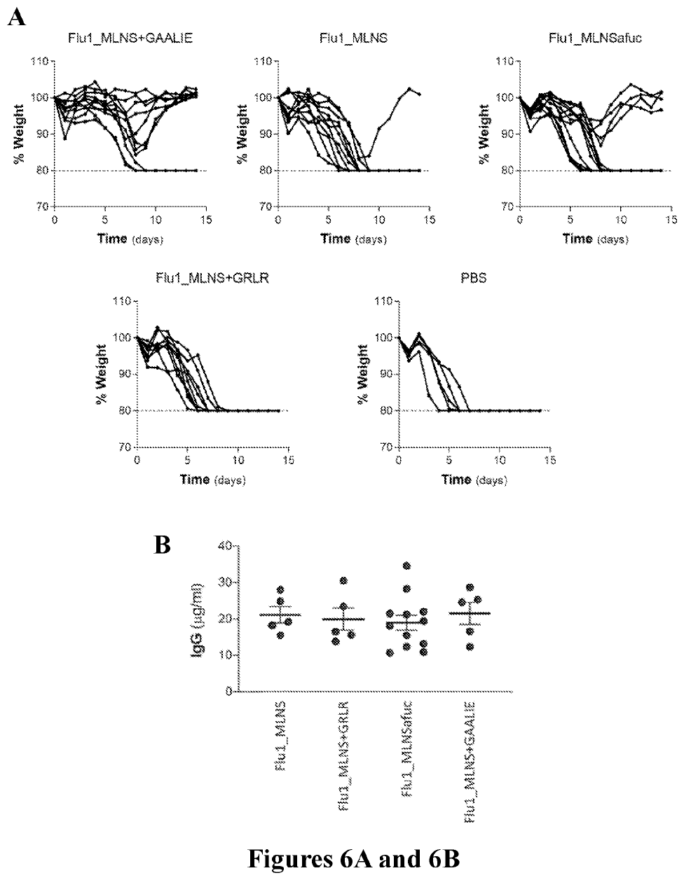

The results are shown in FIGS. 5 and 6. FIG. 5 shows the course of bodyweight after lethal challenge with influenza virus (FIG. 5A) and survival rates after lethal challenge with influenza virus (FIG. 5B). FIG. 6 shows the bodyweight for the individual animals for each group (FIG. 6A) and the serum levels of Flu1 for the four groups of mice receiving distinct antibodies. While the serum Flu1 levels were comparable for the different antibodies (all administered at the same dose of 2 mg/kg), survival rates showed a significant increase for the antibody of the invention Flu1_MLNS+GAALIE, even in comparison to Flu1_MLNSafuc (survival at 2 mg/kg: Flu1_MLNS+GAALIE vs. Flu1_MLNS, p=0.002 and Flu1_MLNS+GAALIE vs. Flu1_MLNSafuc, p=0.04, Log-rank (Mantel-Cox) test). Only in the PBS group and the group which received Flu1_MLNS+GRLR (with abrogated FcγR binding) all animals died.

In summary, the data show that Flu1_MLNS+GAALIE provides superior protection against influenza virus infection. Moreover, as shown in FIG. 6B, the “GAALIE”-mutation (G236A, A330L, and I332E) of antibodies of the invention does not compromise the pharmacokinetics in comparison to Flu1_MLNS (mutations M428L and N434S only) in the presence of ongoing viral replication.

### Example 3: Roles of FcγRIIa and FcγRIIIa in the Increased Protection Against Influenza Infection Mediated by Antibodies of the Invention

In order assess the role of FcγRIIa and FcγRIIIa in the antibody-mediated protection against influenza infection, distinct Fc domain variants of the antibody “Flu1” (comprising the CDR sequences as set forth in SEQ ID NOs 1-6 (or 1-4, 11, and 6, respectively) and the heavy chain variable region (VH) sequence as set forth in SEQ ID NO: 7 and the light chain variable region (VL) sequence as set forth in SEQ ID NO: 8) with distinct affinities for the different FcγRs were directly compared.

The following six “Flu1” Fc variants were tested:

(i) “Flu1_GAALIE”, which comprises the three mutations G236A, A330L and I332E in its heavy chain constant region, no other mutations in constant regions; heavy chain comprising SEQ ID NO: 9, light chain comprising SEQ ID NO: 10;

(ii) “Flu1_wt”, no mutations in constant regions, differs from Flu1_GAALIE only in that it does not contain the three mutations G236A, A330L and I332E; heavy chain comprising SEQ ID NO: 12, light chain comprising SEQ ID NO: 10;

(iii) “Flu1_V11”, which comprises the mutations G237D, P238D, H268D, P271G, and A330R in its heavy chain constant region; heavy chain comprising SEQ ID NO: 17, light chain comprising SEQ ID NO: 10; shows enhanced binding to FcγRIIb, decreased binding to FcγRIIa, and minimal binding to FcγRIIIa/b. (F. Mimoto et al., Protein Engineering, Design and Selection, Volume 26, Issue 10, October 2013, Pages 589-598; Dahan R, et al. Cancer Cell. 2016; 29(6):820-831);

(iv) “Flu1_ALIE”, which comprises the two mutations A330L and I332E in its heavy chain constant region, no other mutations in constant regions; heavy chain comprising SEQ ID NO: 15, light chain comprising SEQ ID NO: 10; shows enhanced binding to FcγRIIIa/b.

(v) “Flu1_afucosylated”, which differs from Flu1_wt in that the oligosaccharides in the Fc region of the antibody do not comprise any fucose sugar units; obtained essentially as described for “Flu1_MLNSafuc” in Example 2; shows enhanced binding to FcγRIIIa/b; and

(vi) “Flu1_GA”, which comprises the mutation G236A in its heavy chain constant region, no other mutations in constant regions; heavy chain comprising SEQ ID NO: 18, light chain comprising SEQ ID NO: 10; shows enhanced binding to FcγRIIa.

Different groups of transgenic C57BL/6 mice lacking all classes of mouse FcγRs, but expressing human FcγRs (FcγR humanized mice; as described in Example 1) received intraperitoneally 2 mg/kg of Flu1_wt, Flu1_GA, Flu1_GAALIE, Flu1_afucosylated, Flu1_ALIE or Flu1_V11. As control, a further group of mice received phosphate-buffered saline (PBS). The experiments were performed essentially as described in Example 1. The antibody (or PBS) was administered intraperitoneally 4 h prior to infection with a lethal dose (5 mLD₅₀) of influenza virus A/Puerto Rico/8/34 H1N1 (PR8). Animals were monitored, and bodyweight was recorded. Blood samples were obtained on days 2 and 3 after infection and serum Flu1 antibody levels were determined (at day 3 after infection) as described in Example 1. In addition, platelets were counted at day 2 post-infection by an automated hematologic analyzer.

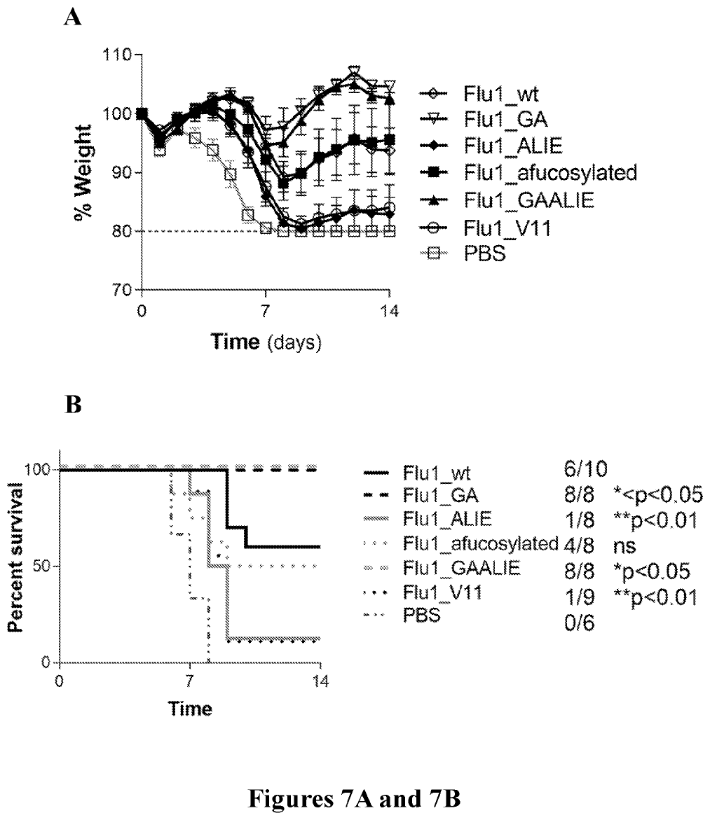

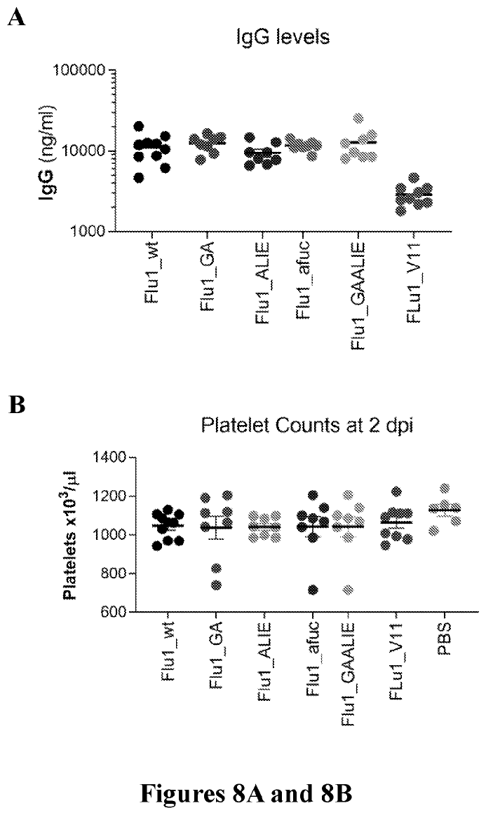

The results are shown in FIGS. 7 and 8. FIG. 7 shows the bodyweights (FIG. 7A) and survival rates (FIG. 7B) for mice treated with distinct Fc variants of antibody Flu1 4 hours prior to infection with PR8 influenza virus. The data show that antibodies of the invention provide superior protection against influenza infection. While the afucosylated antibody (Flu1_afuc) shows a similar course for the bodyweight and survival rates as the wild-type antibody Flu1_wt, antibody Flu1_V11 resulted in decreased bodyweights and decreased survival rates in comparison to the wild-type antibody Flu1_wt.

These results indicate that (i) the enhanced binding to FcγRIIIa (provided by the afucosylated antibody) did not improve efficacy; and (ii) the increased binding to FcγRIIb and decreased or minimal binding to FcγRIIa and FcγRIIIa (provided by Flu1_V11) reduced the antibody's efficacy. In view thereof, increased binding to FcγRIIIa alone may not improve the antibody's efficacy. The superior efficacy of Flu1_GA and Flu1_GAALIE was mediated by increased binding of the antibody to FcγRIIa.

FIG. 8 shows the Flu1 antibody levels determined in serum samples obtained three days after influenza infection (FIG. 8A) and platelet counts two days after influenza infection (FIG. 8B). Except for V11, all Fc variants exhibited essentially the same Flu1 antibody levels. No impact of the Fc variants on platelet counts was detected. Accordingly, no evidence for thrombocytopenia could be observed.

In summary, the data confirm the superior protection of antibodies of the invention and indicate that this effect may be mediated predominantly by increased binding to FcγRIIa.

### Example 4: Increased Protection Against Influenza Infection Mediated by Antibodies of the Invention in Fully Human FcγR and FcRn Mice

Next, various Fc variants of “Flu1” (comprising the CDR sequences as set forth in SEQ ID NOs 1-6 (or 1-4, 11, and 6, respectively) and the heavy chain variable region (VH) sequence as set forth in SEQ ID NO: 7 and the light chain variable region (VL) sequence as set forth in SEQ ID NO: 8) were compared in mice lacking all classes of mouse FcγRs and FcRn, but expressing human FcRn and all classes of human FcγRs (FcγR/FcRn humanized mice). This strain has been generated by crossing the FcγR humanized mouse strain (as described in Smith, P. et al. Proc Natl Acad Sci USA. 2012; 109(16):6181-6) with the FcRn humanized strain (described in Petkova, S. B. et al. Int Immunol 2006; 18(12):1759-69; Roopenian, D. C. et al. Methods Mol Biol 2010; 602:93-104). Mice were screened for FcRn homozygosity, and only FcRn hemizygous mice were included in the experiments.

The following Flu1 Fc variant antibodies were administered at 1 mg/kg i.p. 4 h prior to lethal challenge with 5 mLD₅₀ PR8 influenza virus i.n.:

(i) Flu1_wt (as described in Example 3);

(ii) Flu1_MLNS (as described in Example 1);

(iii) Flu1_GA (as described in Example 3);

(iv) Flu1_MLNS+GA, which contains the mutation G236A and the two mutations

M428L and N434S in the heavy chain constant region, no other mutations in constant regions; heavy chain comprising SEQ ID NO: 19, light chain comprising SEQ ID NO: 10;

(v) Flu1_GAALIE (as described in Example 3); and

(vi) Flu1_MLNS+GAALIE (as described in Example 1).

As control, a further group of mice received phosphate-buffered saline (PBS). The experiments were performed essentially, as described in Example 1. The antibody (or PBS) was administered intraperitoneally at 1 mg/kg 4 h prior to infection with a lethal dose (5 mLD₅₀) of influenza virus A/Puerto Rico/8/34 H1N1 (PR8). Animals were monitored, and bodyweight was recorded. Blood samples were obtained on days 3 and 4 after infection, and Flu1 antibody levels were determined in serum samples obtained from antibody-treated mice (on day 3 after infection) as described in Example 1. In addition, platelets were counted at day 4 post-infection as described in Example 3.

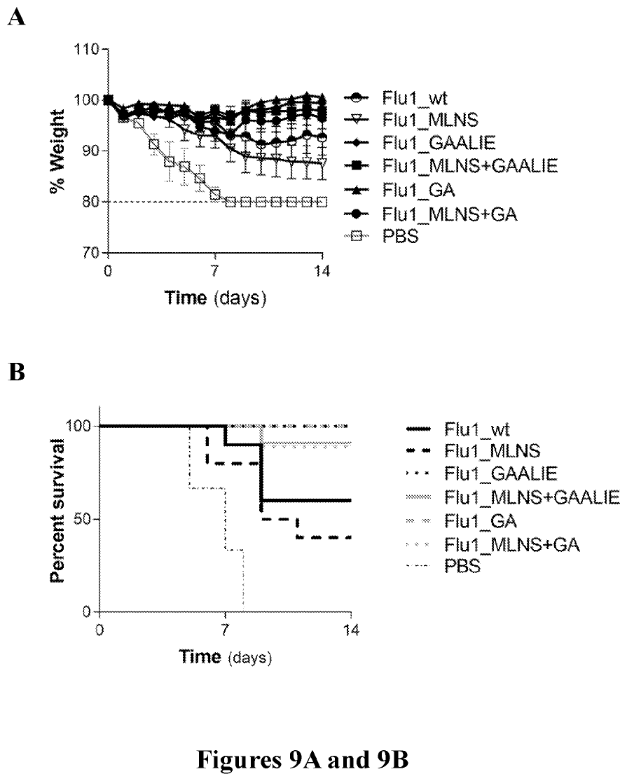

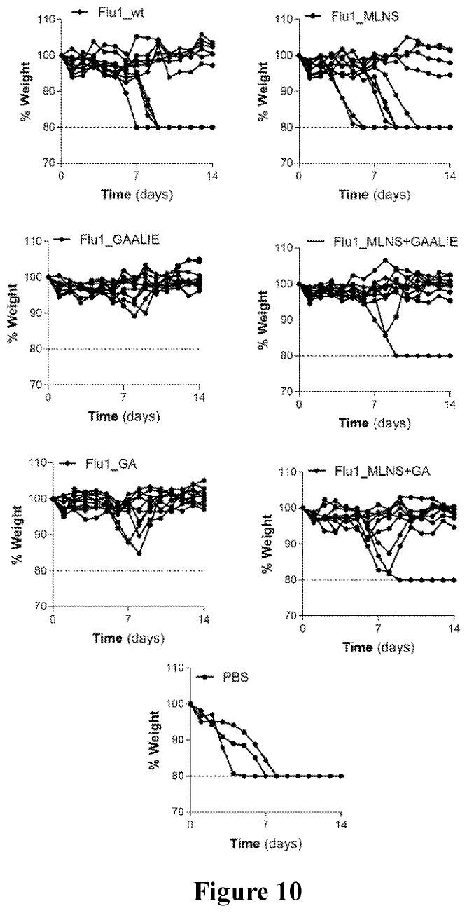

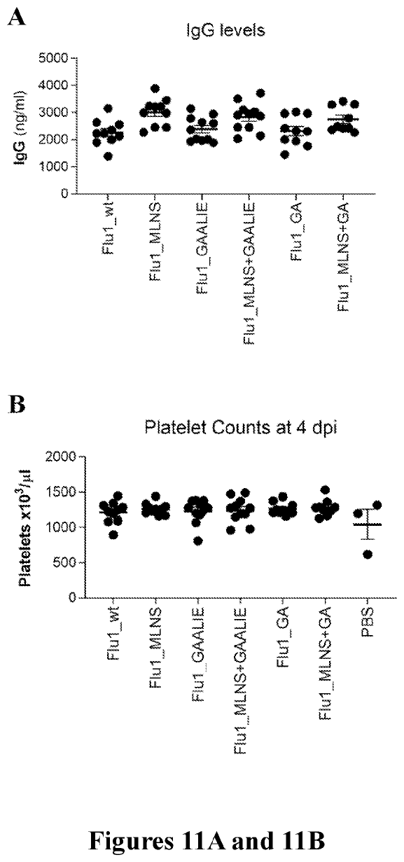

The results are shown in FIGS. 9-11. FIG. 9 shows the bodyweights (FIG. 9A) and survival rates (FIG. 9B) for mice treated with distinct Fc variants of antibody Flu1 four hours prior to infection with PR8 influenza virus. FIG. 10 shows the bodyweight of individual animals for each group. The data show that antibodies of the invention provide superior protection against influenza infection (Flu1_wt vs. Flu1_GA p=0.03; Flu1_wt vs. Flu1_GAALIE p=0.02; Flu1_MLNS vs. Flu1_MLNS+GAALIE p=0.01; Flu1_MLNS vs. Flu1_MLNS+GA p=0.03; Log-rank (Mantel-Cox) test). In particular, the results are consistent with what was observed before in the FcγR humanized mice: an improved activity for the “GA”- and “GAALIE”-variants, independently of the presence of the “MLNS”-mutation. FIG. 11 shows serum Flu1 antibody levels determined on day 3 (FIG. 11A) and platelet counts on day 4 (FIG. 11B). Similarly to the results obtained in the FcγR humanized mice, comparable IgG levels were observed, and no effect of platelet counts could be found. Accordingly, no evidence for thrombocytopenia could be observed.

### Example 5: Increased Protection Against Influenza Infection Mediated by Antibodies of the Invention in Prophylactic Settings

In order to investigate the effects of antibodies of the invention in prophylactic settings, antibodies were administered five days prior to the lethal challenge with influenza virus. The following antibodies were compared in this experiment:

(i) Flu1_wt (as described in Example 3);

(ii) Flu1_MLNS (as described in Example 1);

(iii) Flu1_GAALIE (as described in Example 3); and

(iv) Flu1_MLNS+GAALIE (as described in Example 1).

As control, a further group of mice received phosphate-buffered saline (PBS). The experiments were performed essentially as described in Example 4, with the difference that the antibody was administered 5 days prior to influenza infection. Moreover, in contrast to Example 4, the antibody (or PBS) was administered to female FcγR/FcRn humanized mice intravenously at 0.5 mg/kg 5 days prior to infection with a lethal dose (5 mLD₅₀) of influenza virus A/Puerto Rico/8/34 H1N1 (PR8) i.n. Animals were monitored, and bodyweight was recorded. Blood samples were obtained on the day of infection (i.e., day 0), and serum levels of Flu1 antibodies were determined as described for Example 3.

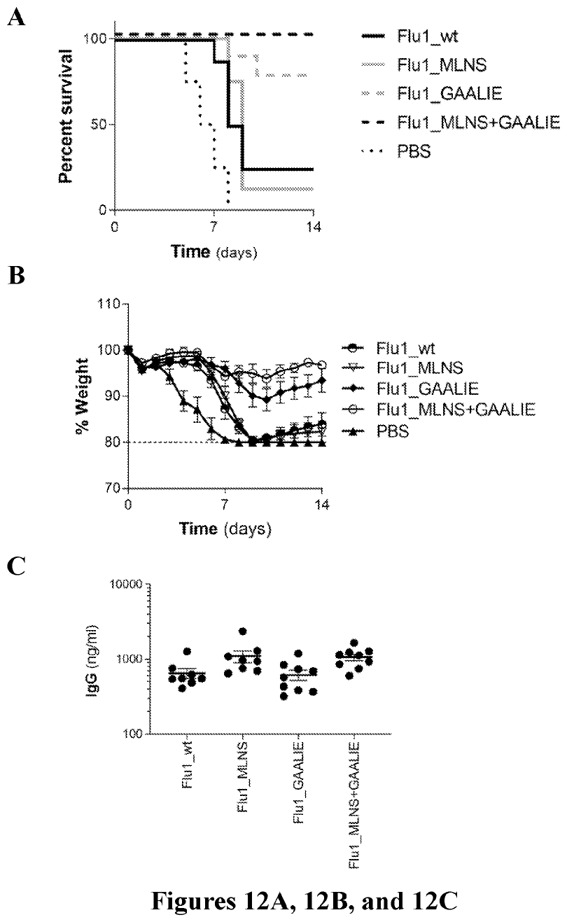

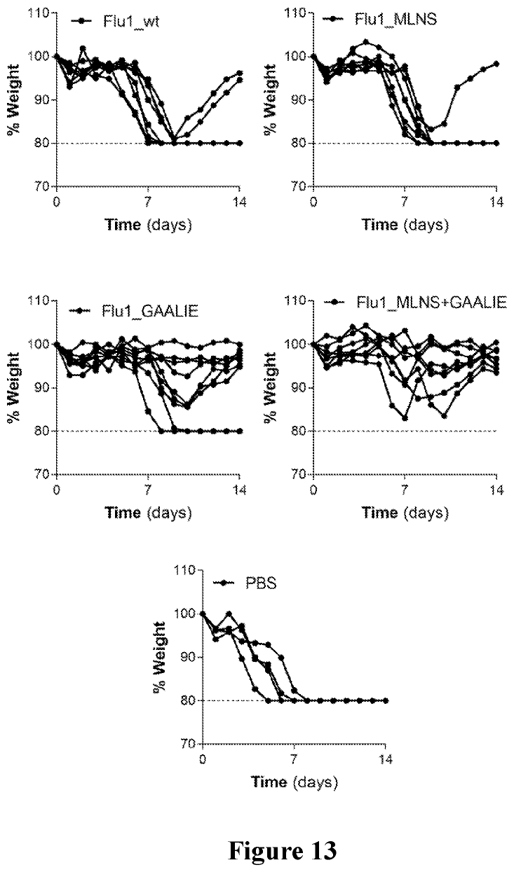

The results are shown in FIGS. 12 and 13. FIG. 12 shows the survival rates (FIG. 12A), bodyweights (FIG. 12B) and serum levels of Flu1 antibodies on the day of virus challenge (FIG. 12C) for mice treated with Flu1_wt, Flu1_MLNS, Flu1_GAALIE, Flu1_MLNS+GAALIE or PBS five days prior to infection with PR8 influenza virus. FIG. 13 shows the bodyweight of individual animals for each group. Mice treated with either Flu1_GAALIE or Flu1_MLNS+GAALIE showed improved protection against influenza infection compared to Flu1_MLNS or Flu1_wt-treated mice (significant survival: Flu1_wt vs. Flu1_GAALIE p=0.02; Flu1_MLNS vs. Flu1_MLNS+GAALIE p=0.0008).

### Example 6: Titration of Antibodies of the Invention in FcγR/FcRn Humanized Mice to Determine the Degree of Enhancement of Protection in Prophylactic Settings

In order to investigate to what extent antibodies of the invention mediate protection against influenza infection in prophylactic settings, antibodies were administered at different doses two days prior to the lethal challenge with influenza virus. The following antibodies were compared in this experiment:

Flu1_MLNS (as described in Example 1);

(ii) Flu1_MLNS+GAALIE (as described in Example 1).

As control, a further group of mice received phosphate-buffered saline (PBS). The experiments were performed essentially as described in Example 5, with the difference that the antibody was administered 2 days prior to influenza infection. Moreover, in contrast to Example 5, the antibody (or PBS) was administered at different doses (ranging from 0.1 mg/kg-1.6 mg/kg) to female FcγR/FcRn humanized mice (age 6-11 weeks old) intravenously 2 days prior to infection with a lethal dose (5 mLD₅₀) of influenza virus A/Puerto Rico/8/34 H1N1 (PR8) i.n. Mice were screened for FcRn homozygosity, and only FcRn homozygous mice were included in the experiments. Animals were monitored, and bodyweight was recorded daily. Blood samples were obtained on the day of infection, and serum levels of Flu1 antibodies were determined as described for Example 3.

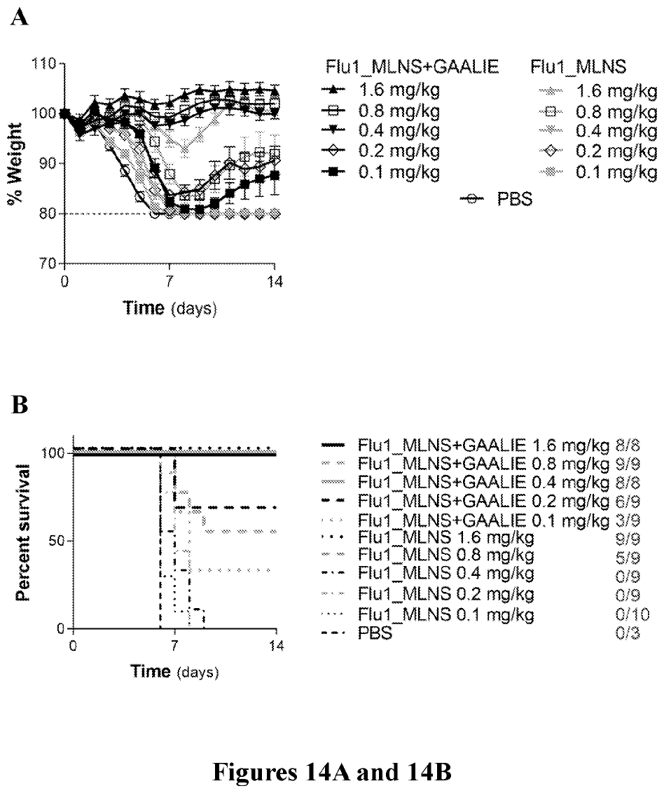

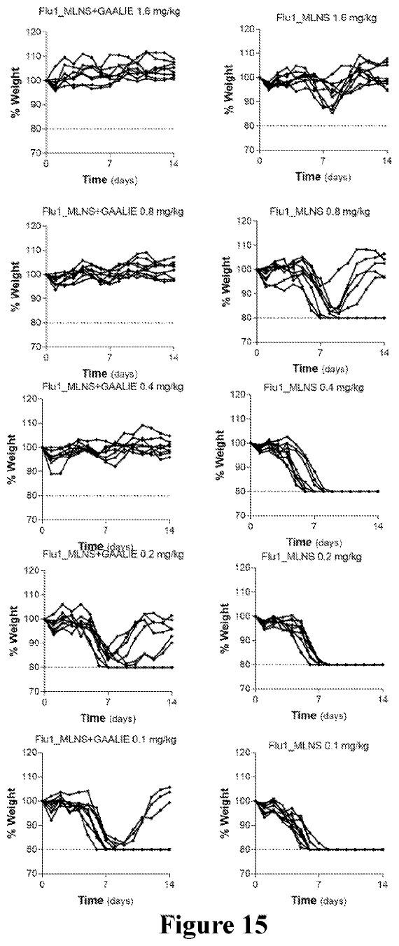

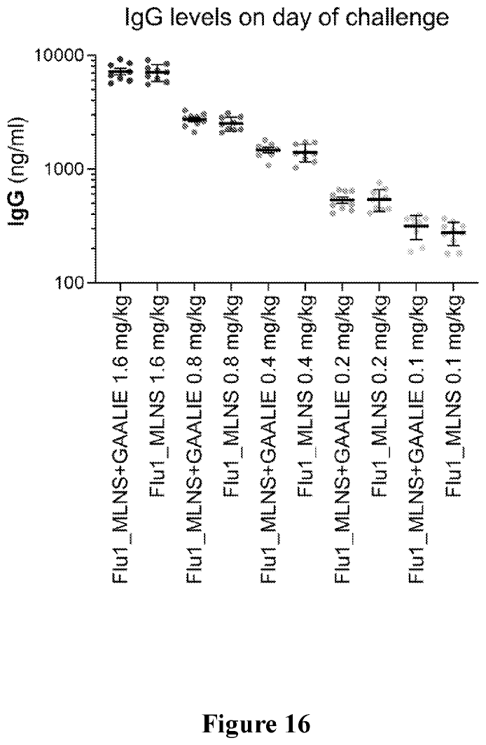

The results are shown in FIGS. 14, 15, and 16. FIG. 14 shows the bodyweights (FIG. 14A and FIG. 15) and survival rates (FIG. 14B) for mice treated with the indicated doses of Flu1_MLNS, Flu1_MLNS+GAALIE, or PBS two days prior to infection with PR8 influenza virus. FIG. 15 shows the bodyweight of individual animals for each group. FIG. 16 shows the serum levels of Flu1 antibodies on the day of virus challenge that were determined as described for Example 3.

Comparison of the protective activity of Flu1_MLNS and Flu1_MLNS+GAALIE across a range of doses revealed superior capacity of the Flu1_MLNS+GAALIE antibody to protect mice from lethal influenza challenge (Flu1_MNLS vs. Flu1_MLNS+GAALIE: p=0.027 at 0.8 mg/kg dose; p 0.000028 at 0.4 mg/kg dose; p=0.0091 at 0.2 mg/kg dose; p=0.0037 at 0.1 mg/kg dose).

### Example 7: Roles of FcγRIIa and FcγRIIIa in the Protection Against Influenza Infection Mediated by Antibodies of the Invention in Therapeutic Settings

In order assess the role of FcγRIIa and FcγRIIIa in the antibody-mediated protection against influenza infection in therapeutic settings, distinct Fc domain variants of the antibody “Flu1” (comprising the CDR sequences as set forth in SEQ ID NOs 1— 6 (or 1— 4, 11, and 6, respectively) and the heavy chain variable region (VH) sequence as set forth in SEQ ID NO: 7 and the light chain variable region (VL) sequence as set forth in SEQ ID NO: 8) with distinct affinities for the different FcγRs were directly compared.

The following six “Flu1” Fc variants were tested:

(i) “Flu1_GAALIE”, as described in Example 3;

(ii) “Flu1_wt”, as described in Example 3;

(iii) “Flu1_afucosylated”, as described in Example 3;

(iv) “Flu1_GA”, as described in Example 3;

(v) “Flu1_MLNS+GRLR, as described in Example 1.

Different groups of transgenic C57BL/6 mice lacking all classes of mouse FcγRs, but expressing human FcγRs (FcγR humanized mice (females, 6-10 weeks old); as described in Example 1) received intraperitoneally 15 mg/kg of Flu1_wt, Flu1_GAALIE, Flu1_afucosylated, or Flu1_MLNS+GRLR. As control, a further group of mice received phosphate-buffered saline (PBS). The experiments were performed essentially as described in Example 1 with the exception that the antibody (or PBS) was administered intraperitoneally three days following infection with a lethal dose (5 mLD₅₀) of influenza virus A/Puerto Rico/8/34 H1N1 (PR8). Animals were monitored, and bodyweight was recorded daily.

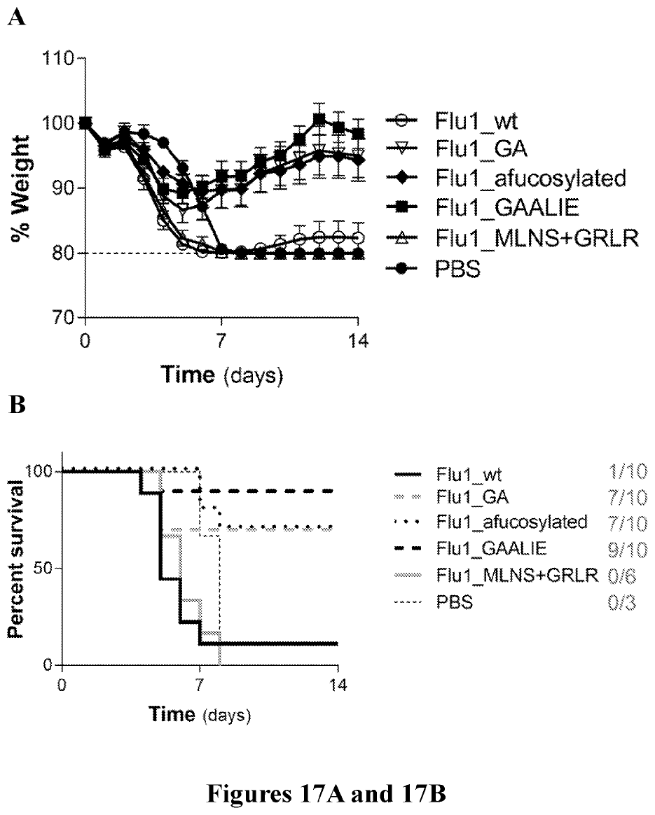

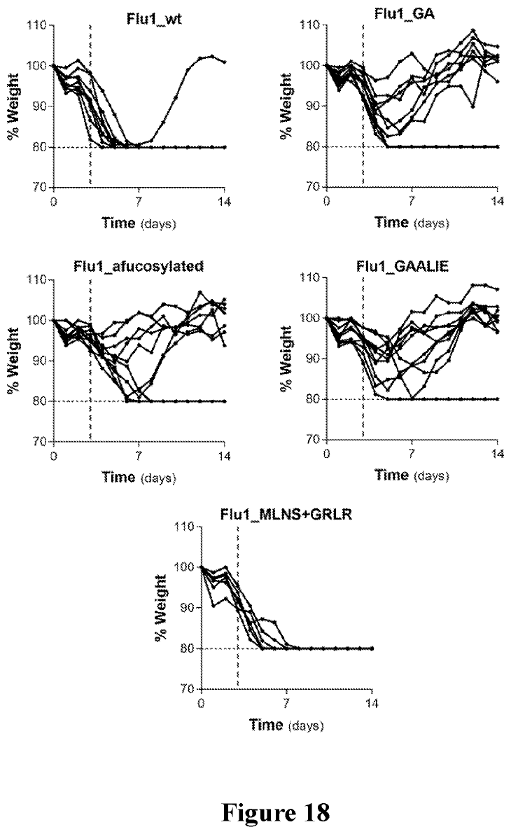

The results are shown in FIGS. 17 and 18. FIG. 17 shows the bodyweights (FIG. 17A) and survival rates (FIG. 17B) for mice treated with distinct Fc variants of antibody Flu1 (15 mg/kg) three days after infection with PR8 influenza virus. FIG. 18 shows the bodyweight of individual animals for each group. The data show that antibodies of the invention provide superior protection against influenza infection in therapeutic settings over Flu1_wt antibodies. As observed in Example 1, Flu1 variants with abrogated FcγR binding (Flu1 MLNS+GRLR) showed minimal protective activity, suggesting that the antibody-mediated protection against influenza infection is dependent on Fc-FcγR interactions. Compared to Flu1_wt, variants with enhanced affinity for either FcγRIIa or FcγRIIIa showed improved therapeutic activity (Flu1_wt vs. Flu1_GA p=0.01; Flu1_wt vs. Flu1_afuc p=0.0009; Flu1_wt vs. Flu1_GAALIE p=0.006).

In summary, the data confirm the superior protection of antibodies of the invention to protect against influenza infection in therapeutic settings and suggest redundant roles for FcγRIIa and FcγRIIIa in the antibody-mediated therapeutic activity against established influenza infection.

### Example 8: Titration of Antibodies of the Invention to Determine the Degree of Enhancement of Protection in Therapeutic Settings

In order to investigate to what extent antibodies of the invention mediate protection against established influenza infection in therapeutic settings, antibodies were administered at different doses to FcγR humanized mice using the experimental conditions described in Example 7. The following antibodies were compared in this experiment:

(i) Flu1_wt (as described in Example 3);

(ii) Flu1_GAALIE (as described in Example 3).

As control, a further group of mice received phosphate-buffered saline (PBS). The experiments were performed essentially, as described in Example 7 The antibody (or PBS) was administered intraperitoneally at different doses (ranging from 5 mg/kg-15 mg/kg) to female FcγR humanized mice (age 6-10 weeks old) 3 days after infection with a lethal dose (5 mLD₅₀) of influenza virus A/Puerto Rico/8/34 H1N1 (PR8) i.n.. Animals were monitored, and bodyweight was recorded daily.

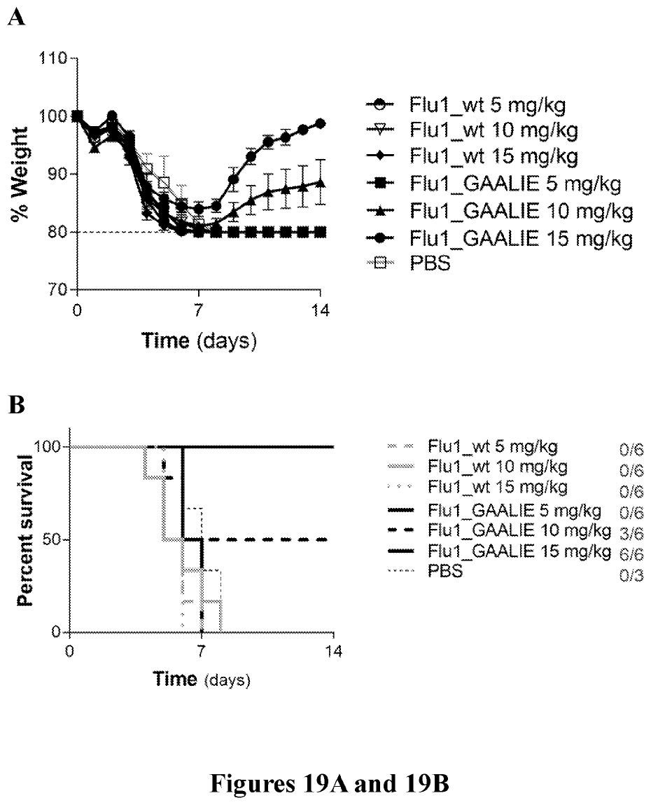

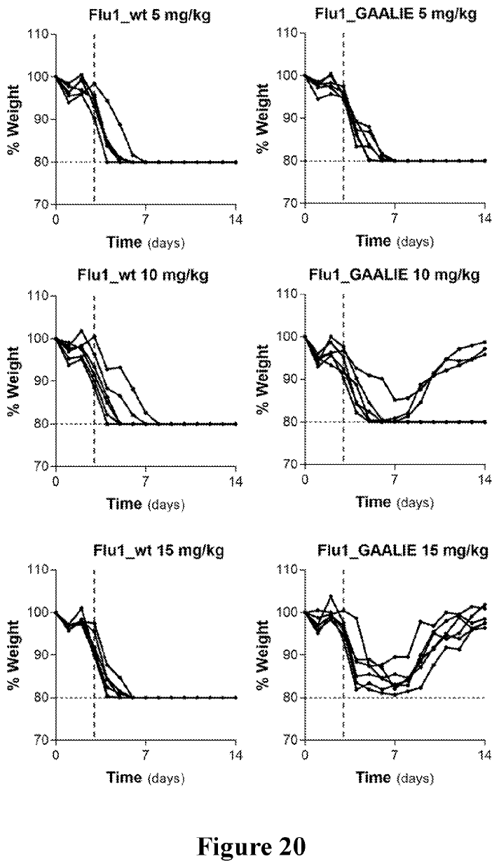

The results are shown in FIGS. 19-20. FIG. 19 shows the bodyweights (FIG. 19A) and survival rates (FIG. 19B) for mice treated with different doses (5-15 mg/kg) of either Flu1_wt or Flu1_GAALIE three days after infection with PR8 influenza virus. FIG. 20 shows the bodyweight of individual animals for each group. The data show that antibodies of the invention provide superior protection against influenza infection in therapeutic settings over Flu1_wt antibodies (Flu1_wt vs. Flu1_GAALIE p=0.0009 at 15 mg/kg dose).

### Example 9: The Role of FcγRIIa and FcγRIIIa in the Antibody-Mediated Protection Against Influenza Infection as Assessed Using the Antibody FI6v3

In order assess the role of FcγRIIa and FcγRIIIa in the antibody-mediated protection against influenza infection, distinct Fc domain variants of the antibody FI6v3 (the heavy chain CDR1, CDR2, and CDR3 sequences as set forth in SEQ ID NO: 26, SEQ ID NO: 27, and SEQ ID NO: 28, respectively, and the light chain CDR1, CDR2, and CDR3 sequences as set forth in: SEQ ID NO: 29, SEQ ID NO: 30, and SEQ ID NO: 31, respectively; and a heavy chain variable region comprising an amino acid sequence set forth in SEQ ID NO: 32 and a light chain variable region comprising the amino acid sequence set forth in SEQ ID NO: 33;) with distinct affinities for the different FcγRs were directly compared.

The following five FI6v3 Fc variants were tested:

“FI6v3_GAALIE”, which comprises the three mutations G236A, A330L and I332E in its heavy chain constant region, no other mutations in constant regions; heavy chain comprising SEQ ID NO: 70, light chain comprising SEQ ID NO: 35;

(ii) “FI6v3_wt”, no mutations in constant regions, differs from FI6v3_GAALIE only in that it does not contain the three mutations G236A, A330L and I332E; heavy chain comprising SEQ ID NO: 66, light chain comprising SEQ ID NO: 35;

(iii) “FI6v3_ALIE”, which comprises the two mutations A330L and I332E in its heavy chain constant region, no other mutations in constant regions; heavy chain comprising SEQ ID NO: 69, light chain comprising SEQ ID NO: 35; shows enhanced binding to FcγRIIIa/b.

(iv) “FI6v3_GRLR”, which comprises the mutations G236R and L328R in its heavy chain constant region, no other mutations in constant regions; heavy chain comprising SEQ ID NO: 67, light chain comprising SEQ ID NO: 35; shows diminished binding to all FcγR classes.

(v) “FI6v3_GA”, which comprises the mutation G236A in its heavy chain constant region, no other mutations in constant regions; heavy chain comprising SEQ ID NO: 68, light chain comprising SEQ ID NO: 35; shows enhanced binding to FcγRIIa.

Different groups of transgenic C57BL/6 mice lacking all classes of mouse FcγRs, but expressing human FcγRs (FcγR humanized mice; as described in Example 1) received intraperitoneally 4 mg/kg of FI6v3_wt, FI6v3_GA, FI6v3_GAALIE, FI6v3_GRLR, or FI6v3_ALIE. As control, a further group of mice received phosphate-buffered saline (PBS). The experiments were performed essentially, as described in Example 1. The antibody (or PBS) was administered intraperitoneally 4 h prior to infection with a lethal dose (5 mLD₅₀) of influenza virus A/Puerto Rico/8/34 H1N1 (PR8). Animals were monitored, and bodyweight was recorded.

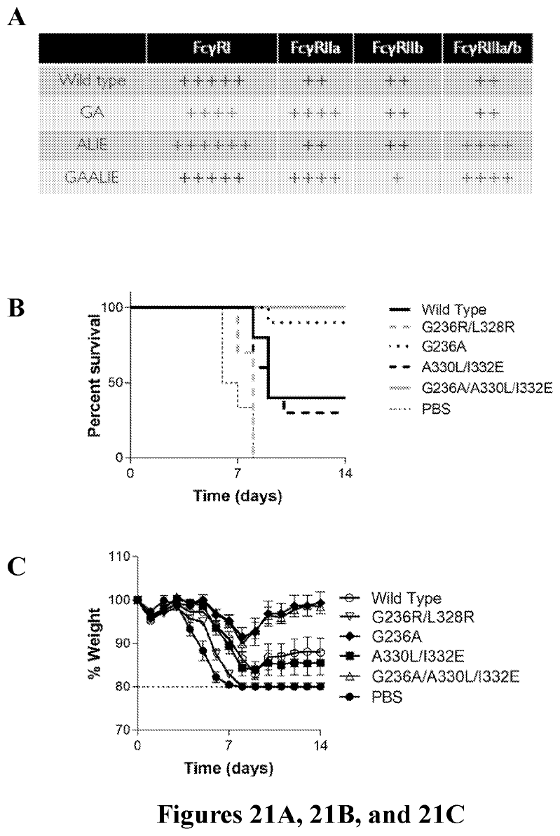

The results are shown in FIG. 21. FIG. 21 shows the survival rates (FIG. 21B) and bodyweights (FIG. 21C) for mice treated with distinct Fc variants of antibody FI6v3 4 hours prior to infection with PR8 influenza virus. The data show that antibodies of the invention provided superior protection against influenza infection. The FI6v3_ALIE antibody showed a similar course for the bodyweight and survival rates as the wild-type antibody FI6v3_wt, indicating that the enhanced binding to FcγRIIIa (provided by the FI6v3_ALIE antibody) did not improve efficacy. In view thereof, increased binding to FcγRIIIa alone may not improve the antibody's efficacy. The superior efficacy of FI6v3_GA and FI6v3_GAALIE was mediated by increased binding of the antibody to FcγRIIa.

In summary, the data confirm the superior protection of antibodies of the invention and indicate that this effect may be mediated predominantly by increased binding to FcγRIIa.

### Example 10: The Role of FcγRIIa and FcγRIIIa in the Antibody-Mediated Protection Against Influenza Infection as Assessed Using the Antibody 3C05

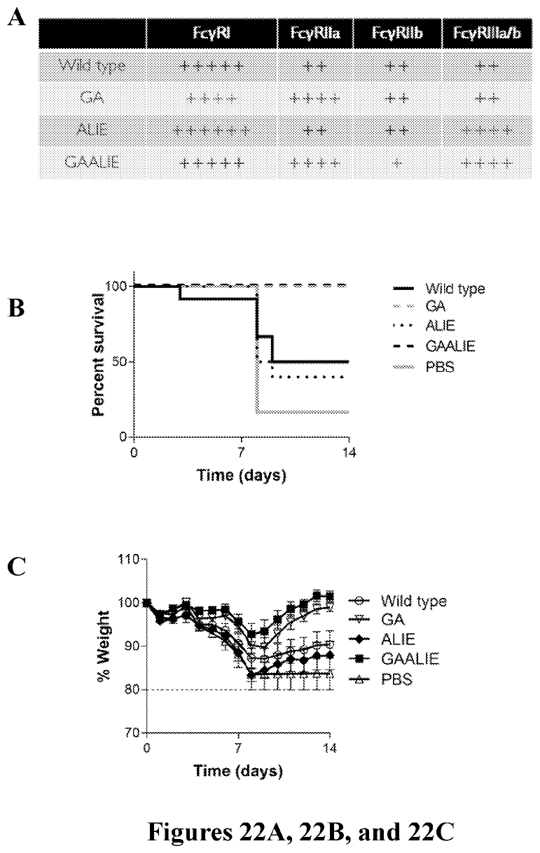

In order assess the role of FcγRIIa and FcγRIIIa in the antibody-mediated protection against influenza infection, distinct Fc domain variants of the antibody 3C05 (the heavy chain CDR1, CDR2, and CDR3 sequences as set forth in SEQ ID NO: 36, SEQ ID NO: 37, and SEQ ID NO: 38, respectively, and the light chain CDR1, CDR2, and CDR3 sequences as set forth in: SEQ ID NO: 39, SEQ ID NO: 40, and SEQ ID NO: 41, respectively; and a heavy chain variable region comprising an amino acid sequence set forth in SEQ ID NO: 42 and a light chain variable region comprising the amino acid sequence set forth in SEQ ID NO: 43;) with distinct affinities for the different FcγRs (FIG. 22A) were directly compared.

The following four 3C05 Fc variants were tested:

(i) “3C05_GAALIE”, which comprises the three mutations G236A, A330L and I332E in its heavy chain constant region, no other mutations in constant regions; heavy chain comprising SEQ ID NO: 75, light chain comprising SEQ ID NO: 45;

(ii) “3C05_wt”, no mutations in constant regions, differs from 3C05_GAALIE only in that it does not contain the three mutations G236A, A330L and I332E; heavy chain comprising SEQ ID NO: 71, light chain comprising SEQ ID NO: 45;

(iii) “3C05_ALIE”, which comprises the two mutations A330L and I332E in its heavy chain constant region, no other mutations in constant regions; heavy chain comprising SEQ ID NO: 74, light chain comprising SEQ ID NO: 45; shows enhanced binding to FcγRIIIa/b.

(iv) “3C05_GA”, which comprises the mutation G236A in its heavy chain constant region, no other mutations in constant regions; heavy chain comprising SEQ ID NO: 73, light chain comprising SEQ ID NO: 45; shows enhanced binding to FcγRIIa.

Different groups of transgenic C57BL/6 mice lacking all classes of mouse FcγRs, but expressing human FcγRs (FcγR humanized mice; as described in Example 1) received intraperitoneally 15 mg/kg of 3C05_wt, 3C05_GA, 3C05_GAALIE, or 3C05_ALIE. As control, a further group of mice received phosphate-buffered saline (PBS). The experiments were performed essentially as described in Example 1. The antibody (or PBS) was administered intraperitoneally 4 h prior to infection with a lethal dose (5 mLD₅₀) of influenza virus A/Netherlands/09 H1N1 (Neth09). Animals were monitored, and bodyweight was recorded.

The results are shown in FIG. 22. FIG. 22 shows the survival rates (FIG. 22B) and bodyweights (FIG. 22C) for mice treated with distinct Fc variants of antibody 3C05 4 hours prior to infection with Neth09 influenza virus. The data show that antibodies of the invention provide superior protection against influenza infection. The 3C05_ALIE antibody shows a similar course for the bodyweight and survival rates as the wild-type antibody 3C05_wt, indicating that the enhanced binding to FcγRIIIa (provided by the 3C05_ALIE antibody) did not improve efficacy. In view thereof, increased binding to FcγRIIIa alone may not improve the antibody's efficacy. The superior efficacy of 3C05_GA and 3C05_GAALIE was mediated by increased binding of the antibody to FcγRIIa.

In summary, the data confirm the superior protection of antibodies of the invention and indicate that this effect may be mediated predominantly by increased binding to FcγRIIa

### Example 11: The Role of FcγRIIa and FcγRIIIa in the Antibody-Mediated Protection Against Influenza Infection as Assessed Using the Antibody TCN032

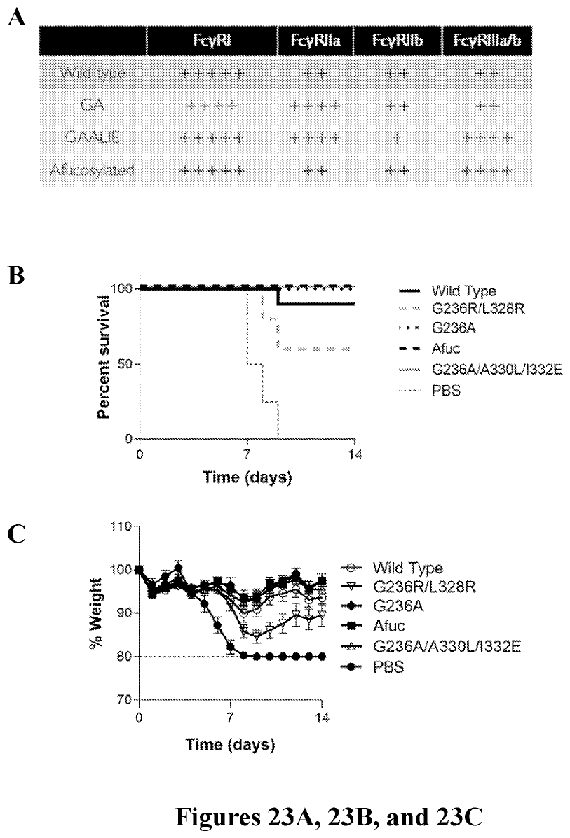

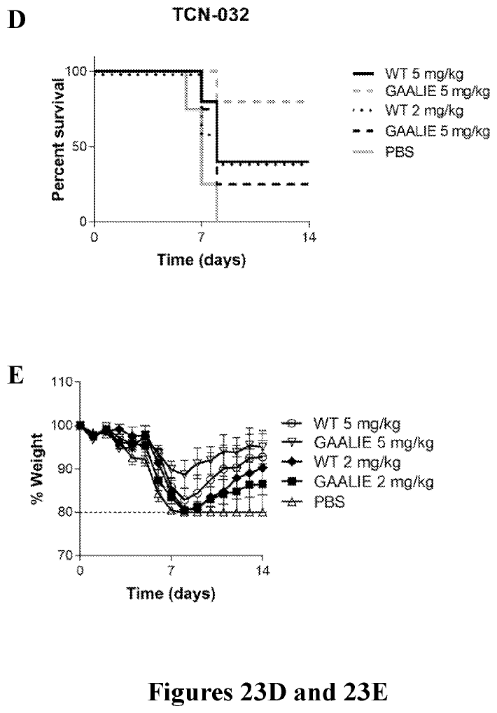

In order assess the role of FcγRIIa and FcγRIIIa in the antibody-mediated protection against influenza infection, distinct Fc domain variants of the antibody TCN032 (the heavy chain CDR1, CDR2, and CDR3 sequences as set forth in SEQ ID NO: 46, SEQ ID NO: 47, and SEQ ID NO: 48, respectively, and the light chain CDR1, CDR2, and CDR3 sequences as set forth in: SEQ ID NO: 49, SEQ ID NO: 50, and SEQ ID NO: 51, respectively; and a heavy chain variable region comprising an amino acid sequence set forth in SEQ ID NO: 52 and a light chain variable region comprising the amino acid sequence set forth in SEQ ID NO: 53;) with distinct affinities for the different FcγRs (FIG. 23A) were directly compared.

The following five TCN032 Fc variants were tested:

(i) “TCN032_GAALIE”, which comprises the three mutations G236A, A330L and I332E in its heavy chain constant region, no other mutations in constant regions; heavy chain comprising SEQ ID NO: 79, light chain comprising SEQ ID NO: 55;

(ii) “TCN032_wt”, no mutations in constant regions, differs from TCN032_GAALIE only in that it does not contain the three mutations G236A, A330L and 1332E; heavy chain comprising SEQ ID NO: 76, light chain comprising SEQ ID NO: 55;

(iii) “TCN032_afuc”, which lacked fucose residues on the Fc-associated glycan, generated as described for Flu1_afuc in Example 1; heavy chain comprising SEQ ID NO: 76, light chain comprising SEQ ID NO: 55; shows enhanced binding to FcγRIIIa/b.

(iv) “TCN032_GA” which comprises the mutation G236A in its heavy chain constant region, no other mutations in constant regions; heavy chain comprising SEQ ID NO: 78, light chain comprising SEQ ID NO: 55; shows enhanced binding to FcγRIIa.

(v) “TCN032_GRLR”, which comprises the mutations G236R and L328R in its heavy chain constant region, no other mutations in constant regions; heavy chain comprising SEQ ID NO: 77, light chain comprising SEQ ID NO: 55; shows diminished binding to all FcγR classes.

Different groups of transgenic C57BL/6 mice lacking all classes of mouse FcγRs, but expressing human FcγRs (FcγR humanized mice; as described in Example 1) received intravenously 10 mg/kg of TCN032_wt, TCN032_GA, TCN032_GAALIE, TCN032_afuc, or TCN032_GRLR. In separate experiments, FcγR humanized mice received intravenously 2 or 5 mg/kg of TCN032_wt or TCN023_GAALIE. As control, a further group of mice received phosphate-buffered saline (PBS). The experiments were performed essentially as described in Example 1. The antibody (or PBS) was administered intravenously 4 h prior to infection with a lethal dose (5 mLD₅₀) of influenza virus A/Puerto Rico/8/34 H1N1 (PR8). Animals were monitored, and bodyweight was recorded.

The results are shown in FIG. 23. FIG. 23 shows the survival rates (FIGS. 23B and 23D) and bodyweights (FIGS. 23C and 23E) for mice treated with distinct Fc variants of antibody TCN032 at the indicated dose: 10 mg/kg for FIGS. 23B and 23C; 2 or 5 mg/kg for FIGS. 23D and 23E) 4 hours prior to infection with PR8 influenza virus. The data show that all the antibodies engineered for increased FcγR affinity (TCN032_GA, TCN032_GAALIE, TCN032_afuc) show a similar course for the bodyweight and survival rates as the wild-type antibody TCN032_wt.

In summary, these data suggest that enhancing the affinity of TCN032 antibodies for human FcγRs does not result in improved antiviral efficacy.

### Example 12: The Role of FcγRIIa and FcγRIIIa in the Antibody-Mediated Protection Against Influenza Infection as Assessed Using the Antibody 14C2

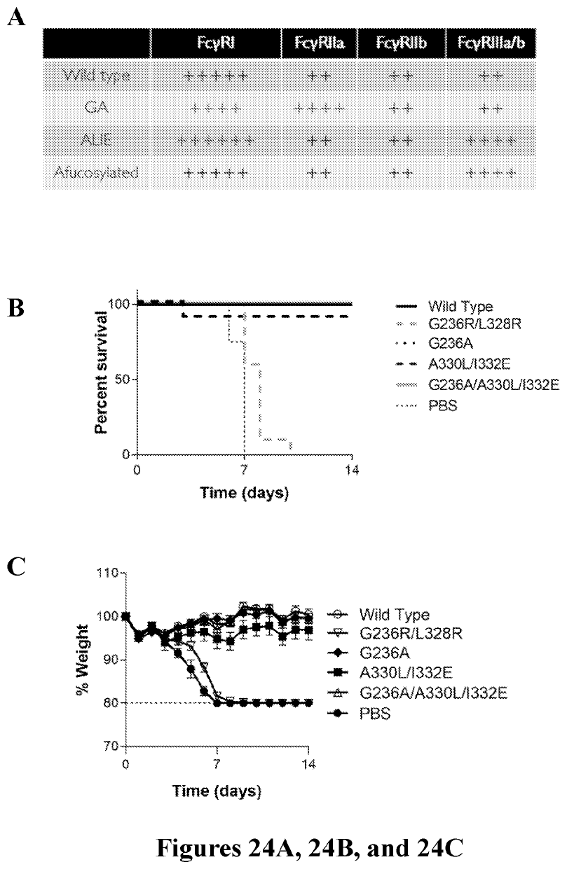

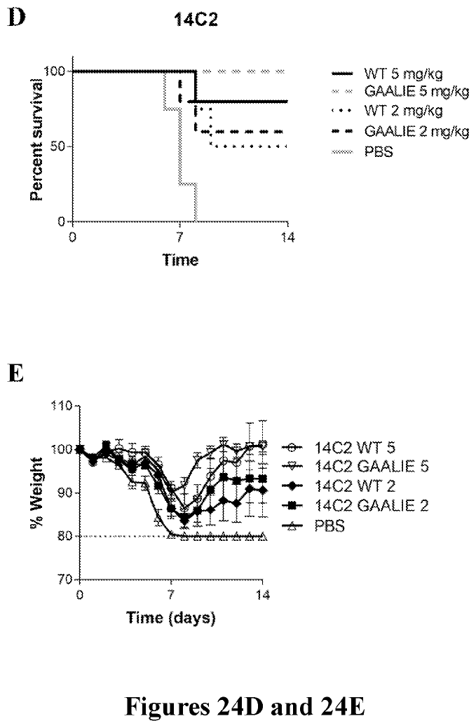

In order assess the role of FcγRIIa and FcγRIIIa in the antibody-mediated protection against influenza infection, distinct Fc domain variants of the antibody 14C2 (the heavy chain CDR1, CDR2, and CDR3 sequences as set forth in SEQ ID NO: 56, SEQ ID NO: 57, and SEQ ID NO: 58, respectively, and the light chain CDR1, CDR2, and CDR3 sequences as set forth in: SEQ ID NO: 59, SEQ ID NO: 60, and SEQ ID NO: 61, respectively; and a heavy chain variable region comprising an amino acid sequence set forth in SEQ ID NO: 62 and a light chain variable region comprising the amino acid sequence set forth in SEQ ID NO: 63) with distinct affinities for the different FcγRs (FIG. 24A) were directly compared.

The following five 14C2 Fc variants were tested:

(i) “14C2_GAALIE”, which comprises the three mutations G236A, A330L and I332E in its heavy chain constant region, no other mutations in constant regions; heavy chain comprising SEQ ID NO: 84, light chain comprising SEQ ID NO: 65;

(ii) “14C2_wt”, no mutations in constant regions, differs from 14C2_GAALIE only in that it does not contain the three mutations G236A, A330L and I332E; heavy chain comprising SEQ ID NO: 80, light chain comprising SEQ ID NO: 65;

(iii) “14C2_ALIE”, which comprises the two mutations A330L and I332E in its heavy chain constant region, no other mutations in constant regions; heavy chain comprising SEQ ID NO: 83, light chain comprising SEQ ID NO: 65; shows enhanced binding to FcγRIIIa/b.

(iv) “14C2_GA”, which comprises the mutation G236A in its heavy chain constant region, no other mutations in constant regions; heavy chain comprising SEQ ID NO: 82, light chain comprising SEQ ID NO: 65; shows enhanced binding to FcγRIIa.

(v) “14C2_GRLR”, which comprises the mutations G236R and L328R in its heavy chain constant region, no other mutations in constant regions; heavy chain comprising SEQ ID NO: 81, light chain comprising SEQ ID NO: 65; shows diminished binding to all FcγR classes.

Different groups of transgenic C57BL/6 mice lacking all classes of mouse FcγRs, but expressing human FcγRs (FcγR humanized mice; as described in Example 1) received intravenously 10 mg/kg of 14C2_wt, 14C2_GA, 14C2_GAALIE, 14C2_ALIE, or 14C2_GRLR.

In separate experiments, FcγR humanized mice received intravenously 2 or 5 mg/kg of 14C2_wt or 14C2_GAALIE. As control, a further group of mice received phosphate-buffered saline (PBS). The experiments were performed essentially, as described in Example 1. The antibody (or PBS) was administered intravenously 4 h prior to infection with a lethal dose (5 mLD₅₀) of influenza virus A/Puerto Rico/8/34 H1N1 (PR8). Animals were monitored, and bodyweight was recorded.

The results are shown in FIG. 24. FIG. 24 shows the survival rates (B, D) and bodyweights (C, E) for mice treated with distinct Fc variants of antibody 14C2 at the indicated dose: 10 mg/kg for FIGS. 24B and 24C; 2 or 5 mg/kg for FIGS. 24D and 24E) 4 hours prior to infection with PR8 influenza virus. The data show that all the antibodies engineered for increased FcγR affinity (14C2_GA, 14C2_GAALIE, 14C2_ALIE) show a similar course for the bodyweight and survival rates as the wild-type antibody 14C2_wt. In summary, these data suggest that enhancing the affinity of 14C2 antibodies for human FcγRs does not result in improved antiviral efficacy.

### Example 13: The Role of FcγRIIa and FcγRIIIa in the Antibody-Mediated Protection Against Influenza Infection as Assessed Using the Antibody 4G05

In order assess the role of FcγRIIa and FcγRIIIa in the antibody-mediated protection against influenza infection, distinct Fc domain variants of the antibody 4G05 (the heavy chain CDR1, CDR2, and CDR3 sequences as set forth in SEQ ID NOs: 85 (nt) and 95 (aa), SEQ ID NOs: 86 (nt) and 96 (aa), and SEQ ID NOs: 87 (nt) and 97 (aa), respectively, and the light chain CDR1, CDR2, and CDR3 sequences as set forth in: SEQ ID NOs: 88 (nt) and 98 (aa), SEQ ID NOs: 89 (nt) and 99 (aa), and SEQ ID NOs: 90 (nt) and 100 (aa), respectively; and a heavy chain variable region comprising the nucleotide sequence and the amino acid sequence set forth in SEQ ID NO: 91 and in SEQ ID NO: 101, respectively and a light chain variable region comprising the nucleotide sequence and the amino acid sequence set forth in SEQ ID NO: 92 and in SEQ ID NO: 102, respectively) with distinct affinities for the different FcγRs were directly compared.

The following five 4G05 Fc variants were tested:

(i) “4G05_GAALIE”, which comprises the three mutations G236A, A330L, and I332E in its heavy chain constant region, no other mutations in constant regions; heavy chain comprising SEQ ID NO: 129, and light chain comprising SEQ ID NO: 104;

(ii) “4G05_wt”, no mutations in constant regions, differs from 4G05_GAALIE only in that it does not contain the three mutations G236A, A330L, and 1332E; heavy chain comprising SEQ ID NO: 125, and light chain comprising SEQ ID NO: 104;

(iii) “4G05_ALIE”, which comprises the two mutations A330L and 1332E in its heavy chain constant region, no other mutations in constant regions; heavy chain comprising SEQ ID NO: 128, and light chain comprising SEQ ID NO: 104; shows enhanced binding to FcγRIIIa/b.

(iv) “4G05_GRLR”, which comprises the mutations G236R and L328R in its heavy chain constant region, no other mutations in constant regions; heavy chain comprising SEQ ID NO: 126, and light chain comprising SEQ ID NO: 104; shows diminished binding to all FcγR classes.

(v) “4G05_GA”, which comprises the mutation G236A in its heavy chain constant region, no other mutations in constant regions; heavy chain comprising SEQ ID NO: 127, and light chain comprising SEQ ID NO: 104; shows enhanced binding to FcγRIIa.

Different groups of transgenic C57BL/6 mice lacking all classes of mouse FcγRs, but expressing human FcγRs (FcγR humanized mice; as described in Example 1) received intravenously 0.5 mg/kg of 4G05_wt, 4G05_GA, 4G05_GRLR, 4G05_GAALIE, or 4G05_ALIE. As control, a further group of mice received phosphate-buffered saline (PBS). The experiments were performed essentially, as described in Example 1. The antibody (or PBS) was administered intravenously 4 h prior to infection with a lethal dose (5 mLD₅₀) of influenza virus A/Netherlands/09 H1N1 (Neth09). Animals were monitored, and bodyweight was recorded.

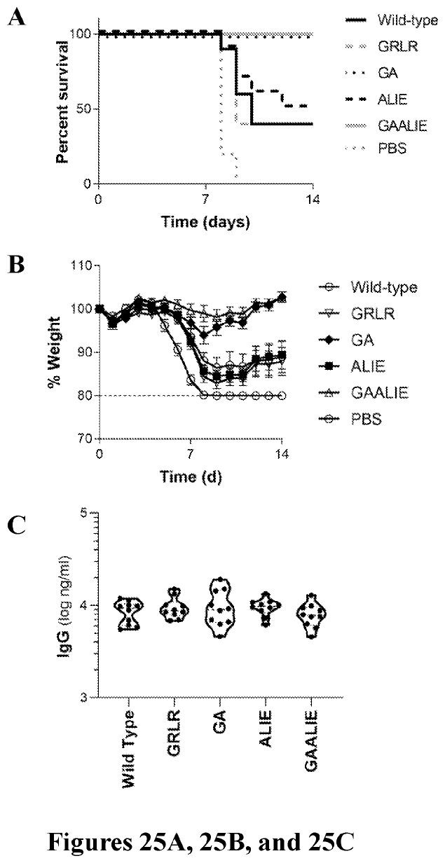

The results are shown in FIG. 25. FIG. 25 shows the survival rates (FIG. 25A), bodyweights (FIG. 25B), and serum levels of 4G05 (FIG. 25C) on day 4 post-infection for mice treated with distinct Fc variants of antibody 4G05 4 hours prior to infection with Neth09 influenza virus. The data show that antibodies of the invention provide superior protection against influenza infection. The 4G05_ALIE antibody shows a similar course for the bodyweight and survival rates as the wild-type antibody 4G05_wt, indicating that the enhanced binding to FcγRIIIa (provided by the 4G05_ALIE antibody) did not improve efficacy.

In view thereof, increased binding to FcγRIIIa alone may not improve the antibody's efficacy. The superior efficacy of 4G05_GA and 4G05_GAALIE was mediated by increased binding of the antibody to FcγRIIa. In summary, the data confirm the superior protection of antibodies of the invention and indicate that this effect may be mediated predominantly by increased binding to FcγRIIa.

### Example 14: The Role of FcγRIIa and FcγRIIIa in the Antibody-Mediated Protection Against Influenza Infection as Assessed Using the Antibody 1A01

In order assess the role of FcγRIIa and FcγRIIIa in the antibody-mediated protection against influenza infection, distinct Fc domain variants of the antibody 1A01 (the heavy chain CDR1, CDR2, and CDR3 sequences as set forth in SEQ ID NOs: 105 (nt) and 115 (aa), SEQ ID NOs: 106 (nt) and 116 (aa), and SEQ ID NOs: 107 (nt) and 117 (aa), respectively, and the light chain CDR1, CDR2, and CDR3 sequences as set forth in: SEQ ID NOs: 108 (nt) and 118 (aa), SEQ ID NOs: 109 (nt) and 119 (aa), and SEQ ID NOs: 110 (nt) and 120 (aa), respectively; and a heavy chain variable region comprising the nucleotide sequence and the amino acid sequence set forth in SEQ ID NO: 111 and in SEQ ID NO: 121, respectively and a light chain variable region comprising the nucleotide sequence and the amino acid sequence set forth in SEQ ID NO: 112 and SEQ ID NO: 122, respectively) with distinct affinities for the different FcγRs were directly compared.

The following five 1A01 Fc variants were tested:

(i) “1A01_GAALIE”, which comprises the three mutations G236A, A330L and I332E in its heavy chain constant region, no other mutations in constant regions; heavy chain comprising SEQ ID NO: 134, light chain comprising SEQ ID NO: 124;

(ii) “1A01_wt”, no mutations in constant regions, differs from 1A01_GAALIE only in that it does not contain the three mutations G236A, A330L and I332E; heavy chain comprising SEQ ID NO: 130, light chain comprising SEQ ID NO: 124;

(iii) “1A01_ALIE”, which comprises the two mutations A330L and I332E in its heavy chain constant region, no other mutations in constant regions; heavy chain comprising SEQ ID NO: 133, light chain comprising SEQ ID NO: 124; shows enhanced binding to FcγRIIIa/b.

(iv) “1A01_GRLR”, which comprises the mutations G236R and L328R in its heavy chain constant region, no other mutations in constant regions; heavy chain comprising SEQ ID NO: 131, light chain comprising SEQ ID NO: 124; shows diminished binding to all FcγR classes.

(v) “1A01_GA”, which comprises the mutation G236A in its heavy chain constant region, no other mutations in constant regions; heavy chain comprising SEQ ID NO: 132, light chain comprising SEQ ID NO: 124; shows enhanced binding to FcγRIIa.

Different groups of transgenic C57BL/6 mice lacking all classes of mouse FcγRs, but expressing human FcγRs (FcγR humanized mice; as described in Example 1) received intravenously 2 mg/kg of 1A01 wt, 1A01_GA, 1A01_GRLR, 1A01_GAALIE, or 1A01_ALIE. As control, a further group of mice received phosphate-buffered saline (PBS). The experiments were performed essentially, as described in Example 1. The antibody (or PBS) was administered intravenously 4 h prior to infection with a lethal dose (5 mLD₅₀) of influenza virus to A/Netherlands/09 H1N1 (Neth09). Animals were monitored, and bodyweight was recorded.

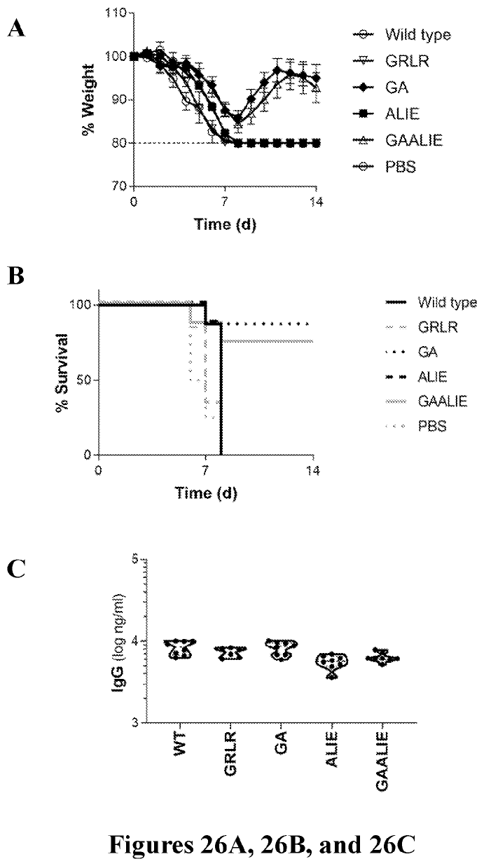

The results are shown in FIG. 26. FIG. 26 shows the bodyweights (FIG. 26A), the survival rates (FIG. 26B), and serum levels of 1A01 (FIG. 26C) on day 4 post-infection for mice treated with distinct Fc variants of antibody 1A01 4 hours prior to infection with Neth09 influenza virus. The data show that antibodies of the invention provide superior protection against influenza infection. The 1A01_ALIE antibody shows a similar course for the bodyweight and survival rates as the wild-type antibody 1A01 wt, indicating that the enhanced binding to FcγRIIIa (provided by the 4G05_ALIE antibody) did not improve efficacy.

In view thereof, increased binding to FcγRIIIa alone may not improve the antibody's efficacy. The superior efficacy of 1A01_GA and 1A01_GAALIE was mediated by increased binding of the antibody to FcγRIIa. In summary, the data confirm the superior protection of antibodies of the invention and indicate that this effect may be mediated predominantly by increased binding to FcγRIIa.

### Example 15: The Impact of FcγRIIa Engagement by Fc Engineered Anti-HA mAbs on DC Maturation and T Cell Activation as Assessed Using the Antibody FI6v3

In order assess the impact of FcγRIIa engagement by Fc engineered anti-HA mAbs on DC maturation and T cell activation, distinct Fc domain variants of the antibody FI6v3 (the heavy chain CDR1, CDR2, and CDR3 sequences as set forth in SEQ ID NO: 26, SEQ ID NO: 27, and SEQ ID NO: 28, respectively, and the light chain CDR1, CDR2, and CDR3 sequences as set forth in: SEQ ID NO: 29, SEQ ID NO: 30, and SEQ ID NO: 31, respectively; and a heavy chain variable region comprising an amino acid sequence set forth in SEQ ID NO: 32 and a light chain variable region comprising the amino acid sequence set forth in SEQ ID NO: 33;) with distinct affinities for the different FcγRs were directly compared.

The following three FI6v3 Fc variants were tested:

(i) “FI6v3_GAALIE”, which comprises the three mutations G236A, A330L, and I332E in its heavy chain constant region, no other mutations in constant regions; heavy chain comprising SEQ ID NO: 70, light chain comprising SEQ ID NO: 35;

(ii) “FI6v3_wt”, no mutations in constant regions, differs from FI6v3_GAALIE only in that it does not contain the three mutations G236A, A330L, and I332E; heavy chain comprising SEQ ID NO: 66, light chain comprising SEQ ID NO: 35;

(iii) “FI6v3_GRLR”, which comprises the mutations G236R and L328R in its heavy chain constant region, no other mutations in constant regions; heavy chain comprising SEQ ID NO: 67, light chain comprising SEQ ID NO: 35; shows diminished binding to all FcγR classes.

Different groups of transgenic C57BL/6 mice lacking all classes of mouse FcγRs, but expressing human FcγRs (FcγR humanized mice; as described in Example 1) received intraperitoneally 3 mg/kg of FI6v3_wt, FI6v3_GAALIE, or FI6v3_GRLR. As control, a further group of mice received phosphate-buffered saline (PBS). The experiments were performed essentially, as described in Example 1. The antibody (or PBS) was administered intraperitoneally 4 h prior to infection with a lethal dose (5 mLD₅₀) of influenza virus A/Puerto Rico/8/34 H1N1 (PR8). Animals were euthanized on day 4, and lungs were harvested to analyze by multi-color flow cytometry the phenotype of DC and T cell populations.

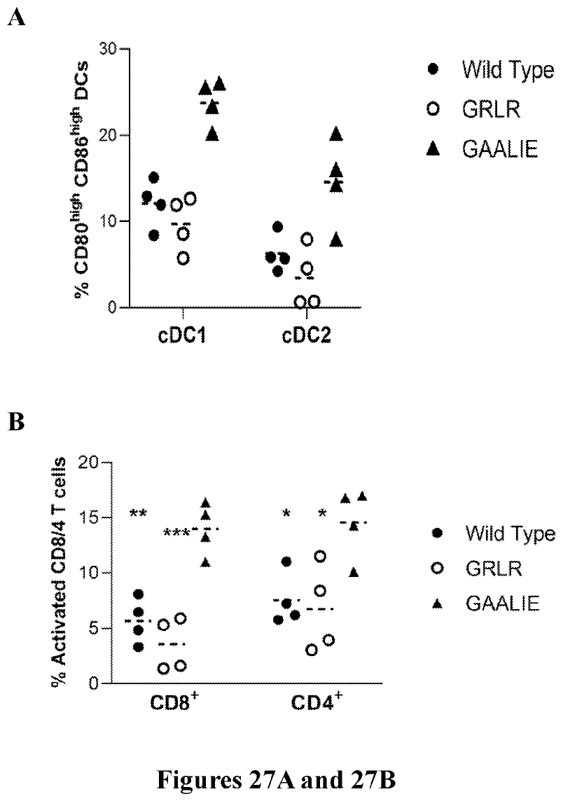

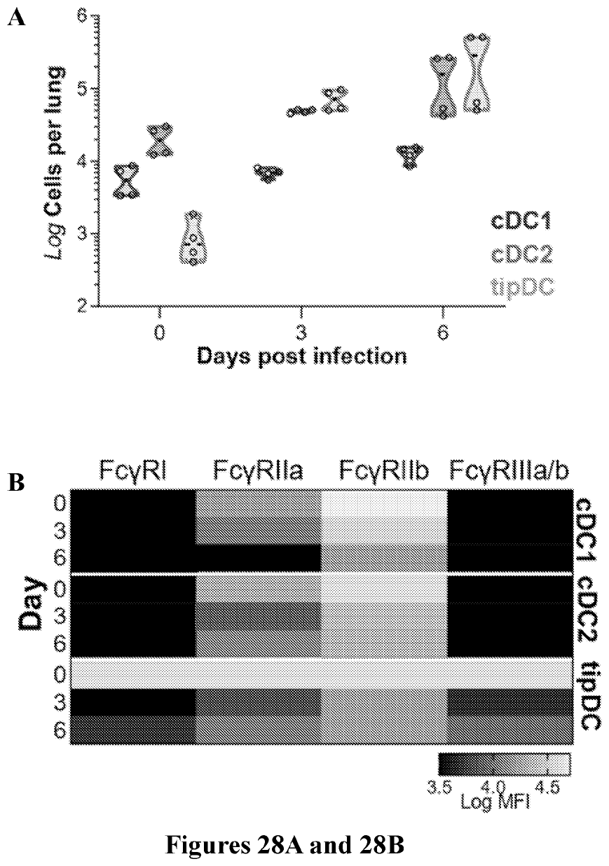

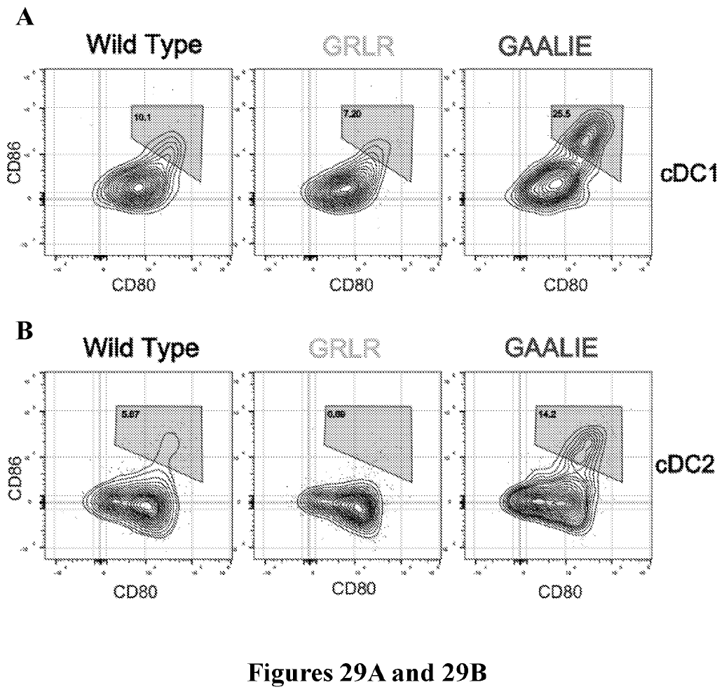

The results are shown in FIGS. 27, 28, and 29. FIG. 27 shows the percentage of mature (defined as CD86hi/CD80hi) cDC1 (CD11c+CD103+CD11b−MHCII+) or cDC2 (CD11b+CD11c+CD103-MHCII+)(FIG. 27A) and activated CD4 and CD8 T cells (defined as CD44+CD69+; FIG. 27B) present on day 4 post-infection in the lungs of FcγR humanized mice treated with distinct Fc variants of the anti-HA stalk antibody FI6v3 (3 mg/kg, i.p.) four hours prior to infection with PR8 H1N1 influenza virus (5 mLD50 i.n.). FIG. 28 shows abundance and FcγR expression profile of DC populations in the lungs of influenza-infected FcγR humanized mice at different time points following infection. To determine the abundance and FcγR expression profile of DC subsets during the course of influenza infection, cohorts of FcγR humanized mice were infected (i.n. with H1N1 PR8; 5 mLD50) and euthanized at different time points following infection (day 0 to day 6). Lungs were homogenized and analyzed by flow cytometry to determine the frequency (FIG. 28A) and FcγR expression profile (FIG. 28B) of the three major DC subsets identified: cDC1 (defined as MHCII+/CD11c+/CD11b−/CD103+), cDC2 (defined as MHCII/CD11c+/CD11b+/CD103−/Gr-1−), and tipDC (TNF-α/iNOS-producing DCs defined as MHCII+/CD11c+/CD11b+/CD103-/Gr-1+). Influenza infection was not associated with any major changes in the number of lung-resident cDC1 and cDC2, whereas tipDCs were almost absent at baseline, but their number increased dramatically upon infection. cDC1 and cDC2 expressed FcγRIIa and FcγRIIb, but they were negative for FcγRIIIa. In contrast, tipDCs expressed FcγRIIa and FcγRIIIa, along with the inhibitory FcγRIIb. FIG. 29 show treatment of FcγR humanized mice with GAALIE variants of anti-HA mAbs is associated with increased frequency of activated DCs. To investigate the impact of enhanced FcγRIIa engagement by GAALIE variants on the maturation status of DCs, FcγR humanized mice were treated with Fc domain variants of the anti-HA stalk mAb FI6v3, exhibiting differential FcγR affinity—wild type IgG1 (baseline FcγR affinity), GRLR (diminished binding to all classes of FcγRs), and GAALIE (enhanced FcγRIIa and FcγRIIIa affinity). Fc domain variants were administered i.p. (3 mg/kg) to FcγR humanized mice 4 h prior to lethal challenge with H1N1 (PR8; 5 mLD50). Mice were euthanized on day 4 and lung-resident DCs were analyzed by flow cytometry. The abundance of mature (defined as CD80high/CD86high) cDC1 (FIG. 29A) and cDC2 (FIG. 29B) was compared between mice treated with the various Fc domain variants of FI6v3.

As shown in FIGS. 27-29, treatment of mice with the FcγRIIa-enhancing variant (GAALIE) prior to influenza challenge, resulted in DC maturation with induction of CD80, CD86 and CD40; an effect that was more pronounced in the cDC1 subset, the DC population specialized for cross-presentation and CD8 T-cell stimulation¹². In contrast, the same mAb (FI6v3) expressed with an Fc modified to abrogate FcγR binding (GRLR) did not result in evidence of DC maturation.

The data show that antibodies of the invention induce augmented DC maturation and T cell activation. The FI6v3_wt antibody shows a similar effect on T cells and DCs as the FI6v3_GRLR. In contrast, FI6v3_GAALIE increases the frequency of mature DCs and activated T cells upon treatment. In summary, the data confirm the superior immunomodulatory activity of antibodies of the invention and indicate that this effect may be mediated predominantly by increased binding to FcγRIIa.

### Example 16: Engagement of FcγRIIa by the GAALIE Variant Induced the Development of Protective CD8 Responses that Contribute to the Antiviral Immunity Against Influenza Infection

In order assess whether engagement of FcγRIIa by the GAALIE variant induces the development of protective CD8 responses that contribute to the antiviral immunity against influenza infection, distinct Fc domain variants of the antibody “Flu1” (comprising the CDR sequences as set forth in SEQ ID NOs: 1-6 (or 1-4, 11, and 6, respectively) and the heavy chain variable region (VH) sequence as set forth in SEQ ID NO: 7 and the light chain variable region (VL) sequence as set forth in SEQ ID NO: 8) with distinct affinities for the different FcγRs were directly compared.

The following two “Flu1” Fc variants were tested:
(i) “Flu1_GAALIE”, as described in Example 3;

(ii) “Flu1_wt”, as described in Example 3;

Different groups of transgenic C57BL/6 mice lacking all classes of mouse FcγRs, but expressing human FcγRs (FcγR humanized mice; as described in Example 1) received intraperitoneally 2 mg/kg of Flu1_wt or Flu1_GAALIE. As control, a further group of mice received phosphate-buffered saline (PBS). The experiments were performed essentially, as described in Example 1. The antibody (or PBS) was administered intraperitoneally 4 h prior to infection with a lethal dose (5 mLD₅₀) of influenza virus A/Puerto Rico/8/34 H1N1 (PR8). Isotype (rat IgG2b; clone LTF-2) or anti-mouse CD8 (clone 2.43) was administered intraperitoneally to mice (150 μg) on day 3 post-infection. Animals were monitored, and bodyweight was recorded.

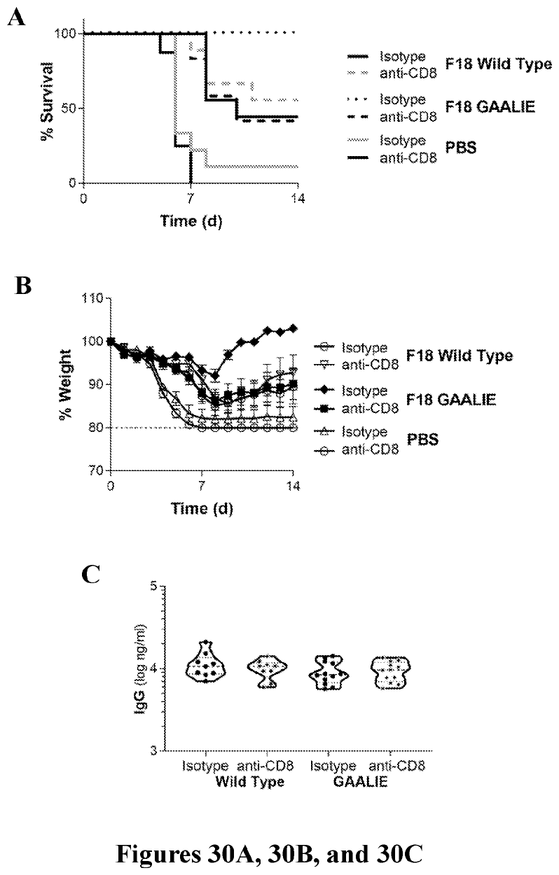

In order to assess the efficacy of CD8 depletion by anti-CD8 antibody treatment, transgenic C57BL/6 mice lacking all classes of mouse FcγRs, but expressing human FcγRs (FcγR humanized mice; as described in Example 1) received intraperitoneally 150 μg of) isotype (rat IgG2b; clone LTF-2) or anti-mouse CD8 (clone 2.43). Blood samples were collected at various time points, and the efficacy of CD8 T cell depletion was assessed by flow cytometry (FIG. 30D).

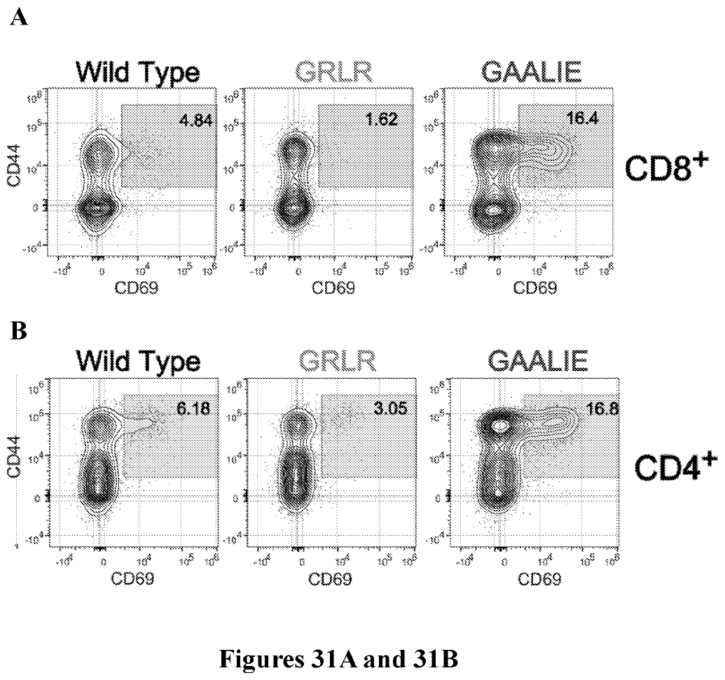

The results are shown in FIGS. 30 and 31. FIG. 30 shows the survival rates (FIG. 30A), the body weights (FIG. 30B), and serum levels of Flu1 (FIG. 30C) on day 4 post-infection for mice treated with either Flu1_wt or Flu1_GAALIE 4 hours prior to infection with PR8 influenza virus, following by administration of isotype or anti-CD8 mAb to deplete CD8 T cells. The data show that the increased protective activity of the antibodies of the invention was mediated by the induction of protective CD8 responses, as depletion of CD8 T cells completely abrogated the protective activity of Flu1_GAALIE. In contrast, CD8 depletion did not influence the sub-optimal protection conferred by wild-type Flu1 (Flu1_wt). FIG. 31 shows treatment of FcγR humanized mice with GAALIE variants of anti-HA stalk mAbs is associated with enhanced activation of CD8+ and CD4+ T cells. To investigate whether the observed increase in the frequency of mature DCs in mice treated with GAALIE variants of antiHA mAbs was associated with enhanced T cell responses, the activation status of CD8 and CD4 T cells was analyzed and compared between mice treated with anti-HA Fc domain variants with differential FcγR affinity (wild type IgG1, GRLR, and GAALIE). Fc domain variants of the antiHA stalk mAb FI6v3 were administered (i.p. 3 mg/kg) to FcγR humanized mice prior to lethal challenge with H1N1 (PR8; 5 mLD50). Mice were euthanized on day 4 post-infection and T-cell populations were analyzed by multicolor flow cytometry. The frequency of activated (defined as CD44hi/CD69+) CD8+(FIG. 31A) and CD4+(FIG. 31B) T cells was compared between mice treated with the various Fc domain variants of FI6v3.

As shown in FIGS. 30 and 31, the GAALIE variant induced enhanced activation of both CD8⁺ and CD4⁺ T cells, while the GRLR variant did not show evidence of robust induction of T cell responses. In summary, the superior efficacy of Flu1_GAALIE was mediated by increased binding of the antibody to FcγRIIa, which in turn induces protective CD8 responses.

### Discussion

Several monoclonal antibodies (mAbs) to influenza virus epitopes from the globular head and the stalk domains of influenza hemagglutinin (HA) and neuraminidase (NA) have been shown to confer broad and potent antiviral activity against diverse influenza strains⁵⁻⁸. These broadly protective mAbs require Fc effector activity to provide full protection from lethal viral challenge, as loss of the capacity of their Fc domain to interact with Fc receptors (FcγRs) expressed on effector leukocytes is associated with reduced in vivo antiviral potency^{5,6}. Although previous studies clearly demonstrated that broadly protective anti-influenza mAbs depend on activating, but not inhibitory FcγRs for activity^{5,6}, the cell types and specific FcγRs that contribute to the antiviral activity of these mAbs remained to be elucidated. The diversity of FcγR expression on immune cells, the structural complexity of the FcγR family and the divergence of these receptors in different species (reviewed in⁹) pose particular challenges in resolving the mechanistic details of how FcγR dependence of anti-influenza antibodies result in enhanced protection in vivo.

To address this problem, a mouse model was previously described in which only human

FcγRs are expressed in a pattern that recapitulates the expression pattern seen in human tissue¹⁰. This in vivo system is combined with anti-influenza antibodies in which the human IgG1 Fc is expressed as a series of variants with selective binding affinity to specific human FcγR (FIG. 21). These antibodies are administered to FcγR humanized mice prior to lethal challenge with influenza (i.n. 5 mLD₅₀) and weight loss and survival are monitored over 14 days. As seen in FIGS. 7 and 21, mice treated with broadly protective mAbs that target the stalk domain of HA (FI6v3 (characterized in⁸) or FY1⁷) show enhanced protection when the Fc is modified to selectively engage the FcγRIIa receptor (GA variant) alone or in combination with enhanced FcγRIIIa binding (GAALIE variant). Enhancing FcγRIIIa binding alone (using two complementary approaches: (i) protein engineering (ALIE variant) or (ii) glycoengineering (afucosylated glycoforms)) does not provide enhanced protection over the wild-type human IgG1, whereas all mAbs fail to protect mice when the Fc is modified to abrogate FcγR binding (GRLR variant) at the selected mAb dose (determined based on titration studies that established the optimal mAb dose required for protection). Additionally, none of these Fc modifications impacted the in vitro neutralization activity or target antigen binding specificity and quantification of the mAb serum levels on day 3 post-infection revealed comparable levels among the different Fc domain variants, suggesting that the observed effects could not be attributed to differential mAb half-life and in vivo stability.

To determine whether the dependence on FcγRIIa for the antiviral protection conferred by anti-HA stalk mAbs also extends to mAbs against other viral epitopes, Fc domain variants for the 4G05 and 1A01 mAbs were generated, which target the globular head of HA and exhibit differential neutralization and HAI activity, as well as for the broadly reactive anti-NA mAb, 3C05⁵. Similar to anti-HA stalk mAbs, Fc variants with enhanced affinity for FcγRIIa (GA or GAALIE variants) demonstrated enhanced protective activity over their wild-type human IgG1 counterparts (FIGS. 22, 25, and 26), suggesting that the FcγR mechanisms by which anti-influenza mAbs confer protection against infection are conserved among mAbs with differential in vitro neutralization potency and epitope specificity.

The above findings clearly demonstrate that while FcγRIIa is the major receptor that drives the protective activity of anti-influenza mAbs, FcγRIIIa has paradoxically limited contribution to the mAb-mediated protection, despite numerous studies that have previously determined that the cytotoxic clearance of malignant or virus-infected cells is predominantly mediated by FcγRIIIa^{2,11}. In addition, despite the abundant expression of FcγRIIIa on alveolar macrophages at baseline as well as the influx of FcγRIIIa-expressing NK cells in response to infection, selective engagement of this receptor did not enhance protection, suggesting that enhancing the clearance of viral particles by alveolar macrophages or killing of infected cells by NK cells does not improve the efficacy of these mAbs in protection against lethal influenza challenge.

FcγRs can either activate (FcγRI, FcγRIIa, and FcγRIIIa) or inhibit (FcγRIIb) cellular responses. Activating FcγRs trigger intracellular signaling subsequent to crosslinking of the extracellular ligand binding domains by IgG immune complexes through either intrinsic cytoplasmic ITAM motifs (FcγRIIa) or γ or ξ chain associated ITAM motifs (FcγRIIIa), recruiting syk family tyrosine kinases (reviewed in¹). Since FcγRIIa and FcγRIII are redundantly expressed on a variety of immune cells, including neutrophils, monocyte/macrophages, and eosinophils, it is unlikely that the unique dependence on FcγRIIa engagement that results in enhanced antiviral protection is mediated by these cells. By contrast, dendritic cells (cDC1 and cDC2 subsets) uniquely express FcγRIIa and the inhibitory receptor FcγRIIb, but not FcγRIIIa, and are found both at baseline and post-infection in the lung (FIG. 28).

To investigate the impact of dendritic cell (DC) FcγRIIa engagement by Fc engineered mAbs on the functional activity of the various DC subsets, lung-resident DCs in influenza-infected FcγR humanized mice that have previously received Fc variants of the anti-HA stalk mAb FI6v3 were analyzed. As shown in FIGS. 14, 19, and 28-31, treatment of mice with the FcγRIIa-enhancing variant (GAALIE) prior to influenza challenge, resulted in DC maturation with induction of CD80, CD86, and CD40; an effect that was more pronounced in the cDC1 subset, the DC population specialized for cross-presentation and CD8 T-cell stimulation¹². In contrast, the same mAb (FI6v3) expressed with an Fc modified to abrogate FcγR binding (GRLR) did not result in evidence of DC maturation. Maturation of DCs and the induction of the accessory molecules CD80, CD86, and CD40 in the virally challenged lung is a prerequisite to the activation of antigen-specific naïve T cells. This would imply that an anti-viral mAb modified to enhance DC maturation through FcγRIIa engagement, can induce an adaptive response to result in the induction of protective T-cell immunity.

To explore this hypothesis, the T-cell responses in the lungs of FcγR humanized mice treated with anti-influenza mAbs with selective FcγR binding properties prior to virus challenge were characterized. As shown in FIGS. 28 and 31, the GAALIE variant induced enhanced activation of both CD8⁺ and CD4⁺ T cells, while the GRLR variant did not show evidence of robust induction of T cell responses. To determine whether the observed induction of T cell activation also contributes to the enhanced protection that was observed with the FcγRIIa binding variants in FIGS. 7, 21-22, and 25-26, the FY1 wild-type and GAALIE variant pre-treatment and viral challenge protocol was repeated, modifying it to include a CD8⁺ cellular depletion step on day 3 post-infection (FIG. 28). Depletion of CD8⁺ T cells resulted in the loss of enhancement of the GAALIE Fc variant, demonstrating that this T-cell population contributes to the improved protection observed for FcγRIIa-enhanced variants (FIG. 28).

Through interactions with effector leukocytes, antibodies against viral antigens have the capacity to enhance disease and contribute to specific histopathologic manifestations. Although this phenomenon, termed antibody-dependent enhancement (ADE), has been demonstrated specifically for flaviviruses, like dengue¹³, clinical experience from severe cases of viral respiratory infections, like influenza and SARS-CoV-2, also supports a pathogenic role for antibodies, which exacerbate disease progression and contribute to severe lung injury through uncontrolled or inappropriate amplification of local inflammatory responses. For example, studies during the 2009 influenza pandemic have shown that severe disease was associated with evidence of IgG-mediated inflammation in the lung parenchyma¹⁴. Likewise, severe cases of COVID-19 disease are often characterized by clinical manifestations resembling cytokine storm syndrome and secondary haemophagocytic lymphohistiocytosis (discussed in¹⁵). Given the capacity of Fc-engineered variants with increased affinity for FcγRIIa to enhance adaptive T-cell responses through activation of FcγRIIa-expressing DCs, it is important to determine whether such variants could also modulate disease pathogenesis through inappropriate amplification of host inflammatory responses that are elicited in response to virus infection. To determine whether FcγRIIa-enhanced variants could lead to severe disease, their in vivo activity in FcγR humanized mice with established influenza infection were assessed. Mice were infected with influenza and 3 days post-infection, FY1 mAb (either wild-type or GAALIE) was administered at different doses (5-15 mg/kg). While wild-type IgG1 FY1 failed to rescue mice from lethal influenza infection, GAALIE variants exhibited a dose-dependent therapeutic benefit, suggesting that enhancing FcγRIIa engagement has no pathogenic consequences, rather it provides meaningful and robust protection from established infection (FIG. 19).

In addition to their therapeutic potential, mAbs engineered for enhanced FcγRIIa affinity could provide long-term prophylaxis from influenza infection, especially when combined with Fc domain mutations (e.g., the LS (M428S/L434S) variant¹⁶) that increase affinity for human FcRn and extend IgG half-life in vivo¹⁶. Using a mouse model of mAb-mediated prophylaxis of influenza infection, the capacity of LS (enhanced for FcRn) and GAALIE/LS (enhanced for FcRn, FcγRIIa and FcγRIIIa) variants of FY1 to protect FcγR/FcRn humanized mice from influenza infection were compared. At all doses tested (0.1-1.6 mg/kg), GAALIE/LS variants demonstrated superior protective activity over their LS counterparts (FIG. 14). Additionally, quantification of the protective activity of the two FY1 Fc variants over a wide range of doses revealed that the GAALIE/LS variant exhibited at least 5.5-fold improvement in in vivo antiviral potency, suggesting that Fc engineering for enhanced affinity to specific FcγR represents a promising approach that could substantially improve the clinical efficacy of antiviral mAbs.

IgG antibodies are capable of mediating pleiotropic effects, resulting from the diversity of Fc binding molecules that engage the Fc domain. The Fc domain is structurally diverse, the consequence of subclasses and Fc glycosylation, resulting in differential Fc receptor binding activities for various Fc structural variants (reviewed in¹). This natural heterogeneity contributes to the efficacy of polyclonal IgG responses to viral infections, providing a mechanism for the recognition of diverse viral epitopes and triggering multiple effector pathways. The development of mAbs for the selective binding to specific neutralizing viral epitopes can now be coupled to Fc modifications to facilitate the engagement of specific FcγRs to optimize the potency of these therapeutics. It has been presumed that, as has been demonstrated for anti-tumor antibody therapeutics, enhancing engagement of innate effector pathways resulting in the phagocytosis of cells by macrophages (ADCP) and the killing of cells by NK cells (ADCC) through FcγRIIIa crosslinking resulting in increased therapeutic efficacy, the same would be true for anti-viral protection. However, this does not appear to be the case. Antibody treatment of HIV infection has been shown to induce a CD8⁺ response both in chronically infected macaques and humans which contributes to the control of viremia^{17,18}. The results of this study demonstrate that selective engagement of the activating FcγR on DCs, FcγRIIa, by a variety of anti-influenza mAbs results in the induction of a protective CD8⁺ response, mechanistically similar to the “vaccinal” response that was observed for anti-tumor antibody treatment¹¹. The ability of an antibody to not only couple to innate effector responses through its Fc domain, but also induce an adaptive response by engaging and activating dendritic cells, provides a potent new approach to the design of therapeutic antibodies for the prevention and treatment of viral diseases. This approach to Fc engineering is particularly relevant to pandemic viruses, like influenza and SARS-CoV2. Neutralizing antibodies to these viruses, engineered to enhance DC activation and CD8⁺ T-cell responses, as shown here for the GAALIE variant, are predicted to provide significant enhancement of protection by stimulating a variety of synergistic immunological pathways.

### REFERENCES

1 Bournazos, S., Wang, T. T., Dahan, R., Maamary, J. & Ravetch, J. V. Signaling by Antibodies: Recent Progress. Annu Rev Immunol 35, 285-311, doi:10.1146/annurev-immunol-051116-052433 (2017).
2 Lu, C. L. et al. Enhanced clearance of HIV-1-infected cells by broadly neutralizing antibodies against HIV-1 in vivo. Science 352, 1001-1004, doi:10.1126/science.aaf1279 (2016).
3 Bournazos, S., DiLillo, D. J. & Ravetch, J. V. The role of Fc-FcγR interactions in IgG-mediated microbial neutralization. J Exp Med 212, 1361-1369, doi:10.1084/jem.20151267 (2015).
4 Bournazos, S. et al. Broadly Neutralizing Anti-HIV-1 Antibodies Require Fc Effector Functions for In Vivo Activity. Cell 158, 1243-1253, doi:10.1016/j.ce11.2014.08.023 (2014).
5 DiLillo, D. J., Palese, P., Wilson, P. C. & Ravetch, J. V. Broadly neutralizing anti-influenza antibodies require Fc receptor engagement for in vivo protection. J Clin Invest 126, 605-610, doi:10.1172/JCI84428 (2016).
6 DiLillo, D. J., Tan, G. S., Palese, P. & Ravetch, J. V. Broadly neutralizing hemagglutinin stalk-specific antibodies require FcgammaR interactions for protection against influenza virus in vivo. Nat Med 20, 143-151, doi:10.1038/nm.3443 (2014).
7 Kallewaard, N. L. et al. Structure and Function Analysis of an Antibody Recognizing All Influenza A Subtypes. Cell 166, 596-608, doi:10.1016/j.ce11.2016.05.073 (2016).
8 Corti, D. et al. A neutralizing antibody selected from plasma cells that binds to group 1 and group 2 influenza A hemagglutinins. Science 333, 850-856, doi:10.1126/science.1205669 (2011).
9 Bournazos, S. IgG Fc Receptors: Evolutionary Considerations. Curr Top Microbiol Immunol, doi:10.1007/82 2019 149 (2019).
10 Smith, P., DiLillo, D. J., Bournazos, S., Li, F. & Ravetch, J. V. Mouse model recapitulating human Fcgamma receptor structural and functional diversity. Proc Natl Acad Sci USA 109, 6181-6186, doi:10.1073/pnas.1203954109 (2012).
11 DiLillo, D. J. & Ravetch, J. V. Differential Fc-Receptor Engagement Drives an Anti-tumor Vaccinal Effect. Cell 161, 1035-1045, doi:10.1016/j.ce11.2015.04.016 (2015).
12 Eisenbarth, S. C. Dendritic cell subsets in T cell programming: location dictates function. Nat Rev Immunol 19, 89-103, doi:10.1038/s41577-018-0088-1 (2019).
13 Wang, T. T. et al. IgG antibodies to dengue enhanced for FcγRIIIA binding determine disease severity. Science 355, 395-398, doi:10.1126/science.aai8128 (2017).
14 Monsalvo, A. C. et al. Severe pandemic 2009 H1N1 influenza disease due to pathogenic immune complexes. Nat Med 17, 195-199, doi:10.1038/nm.2262 (2011).
15 Mehta, P. et al. COVID-19: consider cytokine storm syndromes and immunosuppression. Lancet 395, 1033-1034, doi:10.1016/S0140-6736(20)30628-0 (2020).
16 Zalevsky, J. et al. Enhanced antibody half-life improves in vivo activity. Nat Biotechnol 28, 157-159, doi:10.1038/nbt.1601 (2010).
17 Nishimura, Y. et al. Early antibody therapy can induce long-lasting immunity to SHIV. Nature 543, 559-563, doi:10.1038/nature21435 (2017).
18 Niessl, J. et al. Combination anti-HIV-1 antibody therapy is associated with increased virus-specific T cell immunity. Nat Med 26, 222-227, doi:10.1038/s41591-019-0747-1 (2020).
19 Petkova, S. B. et al. Enhanced half-life of genetically engineered human IgG1 antibodies in a humanized FcRn mouse model: potential application in humorally mediated autoimmune disease. Int Immunol 18, 1759-1769, doi:10.1093/intimm/dx1110 (2006).
20 Roopenian, D. C., Christianson, G. J. & Sproule, T. J. Human FcRn transgenic mice for pharmacokinetic evaluation of therapeutic antibodies. Methods Mol Biol 602, 93-104, doi:10.1007/978-1-60761-058-8 6 (2010).
21 Weitzenfeld, P., Bournazos, S. & Ravetch, J. V. Antibodies targeting sialyl Lewis A mediate tumor clearance through distinct effector pathways. J Clin Invest 129, 3952-3962, doi:10.1172/JCI128437 (2019).
22 Okeley, N. M. et al. Development of orally active inhibitors of protein and cellular fucosylation. Proc Natl Acad Sci USA 110, 5404-5409, doi:10.1073/pnas.1222263110 (2013).
23 He, W., Mullarkey, C. E. & Miller, M. S. Measuring the neutralization potency of influenza A virus hemagglutinin stalk/stem-binding antibodies in polyclonal preparations by microneutralization assay. Methods 90, 95-100, doi:10.1016/j.ymeth.2015.04.037 (2015).
24 Kaufmann, L. et al. An Optimized Hemagglutination Inhibition (HI) Assay to Quantify Influenza-specific Antibody Titers. J Vis Exp, doi:10.3791/55833 (2017).
25 Samusik, N., Good, Z., Spitzer, M. H., Davis, K. L. & Nolan, G. P. Automated mapping of phenotype space with single-cell data. Nat Methods 13, 493-496, doi:10.1038/nmeth 3863 (2016).

TABLE OF SEQUENCES AND SEQ ID NUMBERS (SEQUENCE LISTING)

| | | |
|---|---|---|
| SEQ ID NO | Sequence | Remarks |

Amino acid sequences

| | | |
|---|---|---|
| Flu1_GAALIE | | |
| SEQ ID NO: 1 | GDSVSSNNAV | CDRH1 |
| SEQ ID NO: 2 | TYYRSKWYN | CDRH2 |
| SEQ ID NO: 3 | VRSGHITVFGVNVDAFDM | CDRH3 |
| SEQ ID NO: 4 | QSLSSYLH | CDRL1 |
| SEQ ID NO: 5 | AAS | CDRL2 |
| SEQ ID NO: 6 | QQSRT | CDRL3 |
| SEQ ID NO: 7 | QVQLQESGPGLVKPSQTLSLTCAISGDSVSSNNA | VH |
| | VWNWIRQSPSRGLEWLGRTYYRSKWYNDYAES | |
| | VKSRITVNPDTSKNQFSLHLKSVTPEDTAVFYCV | |
| | RSGHITVFGVNVDAFDMWGQGTMVTVSS | |
| SEQ ID NO. 8 | DIQMTQSPSSLSASVGDRVTITCRTSQSLSSYLH | VL |
| | WYQQKPGKAPKLLIYAASSLQSGVPSRFSGSGSG | |
| | TDFTLTISSLQPEDFATYYCQQSRTFGQGTKVEIK | |
| SEQ ID NO: 9 | QVQLQESGPGLVKPSQTLSLTCAISGDSVSSNNAV | Heavy chain |
| | WNWIRQSPSRGLEWLGRTYYRSKWYNDYAESV | |
| | KSRITVNPDTSKNQFSLHLKSVTPEDTAVFYCVRS | |
| | GHITVFGVNVDAFDMWGQGTMVTVSSASTKGPS | |
| | VFPLAPSSKSTSGGTAALGCLVKDYFPEPVTVSW | |
| | NSGALTSGVHTFPAVLQSSGLYSLSSVVTVPSSSL | |
| | GTQTYICNVNHKPSNTKVDKRVEPKSCDKTHTCP | |
| | PCPAPELLAGPSVFLFPPKPKDTLMISRTPEVTCV | |
| | VVDVSHEDPEVKFNWYVDGVEVHNAKTKPREE | |
| | QYNSTYRVVSVLTVLHQDWLNGKEYKCKVSNK | |
| | ALPLPEEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVM | |
| | HEALHNHYTQKSLSLSPGK | |
| SEQ ID NO: 10 | DIQMTQSPSSLSASVGDRVTITCRTSQSLSSYLHW | Light chain |
| | YQQKPGKAPKLLIYAASSLQSGVPSRFSGSGSGTD | |
| | FTLTISSLQPEDFATYYCQQSRTFGQGTKVEIKRT | |
| | VAAPSVFIFPPSDEQLKSGTASVVCLLNNFYPREA | |
| | KVQWKVDNALQSGNSQESVTEQDSKDSTYSLSS | |
| | TLTLSKADYEKHKVYACEVTHQGLSSPVTKSFNR | |
| | GEC | |
| SEQ ID NO. 11 | AASS | CDRL2 long |
| Flu1_wt | | |
| SEQ ID NO: 12 | QVQLQESGPGLVKPSQTLSLTCAISGDSVSSNNA | Heavy chain |
| | VWNWIRQSPSRGLEWLGRTYYRSKWYNDYAES | |
| | VKSRITVNPDTSKNQFSLHLKSVTPEDTAVFYCV | |
| | RSGHITVFGVNVDAFDMWGQGTMVTVSSASTK | |
| | GPSVFPLAPSSKSTSGGTAALGCLVKDYFPEPVTV | |
| | SWNSGALTSGVHTFPAVLQSSGLYSLSSVVTVPS | |
| | SSLGTQTYICNVNHKPSNTKVDKRVEPKSCDKTH | |
| | TCPPCPAPELLGGPSVFLFPPKPKDTLMISRTPEVT | |
| | CVVVDVSHEDPEVKFNWYVDGVEVHNAKTKPR | |
| | EEQYNSTYRVVSVLTVLHQDWLNGKEYKCKVS | |
| | NKALPAPIEKTISKAKGQPREPQVYTLPPSREEMT | |
| | KNQVSLTCLVKGFYPSDIAVEWESNGQPENNYK | |
| | TTPPVLDSDGSFFLYSKLTVDKSRWQQGNVFSCS | |
| | VMHEALHNHYTQKSLSLSPGK | |
| Flu1_MLNS | | |
| SEQ ID NO: 13 | QVQLQESGPGLVKPSQTLSLTCAISGDSVSSNNA | Heavy chain |
| | VWNWIRQSPSRGLEWLGRTYYRSKWYNDYAES | |
| | VKSRITVNPDTSKNQFSLHLKSVTPEDTAVFYCV | |
| | RSGHITVFGVNVDAFDMWGQGTMVTVSSASTK | |
| | GPSVFPLAPSSKSTSGGTAALGCLVKDYFPEPVTV | |
| | SWNSGALTSGVHTFPAVLQSSGLYSLSSVVTVPS | |
| | SSLGTQTYICNVNHKPSNTKVDKRVEPKSCDKTH | |
| | TCPPCPAPELLGGPSVFLFPPKPKDTLMISRTPEVT | |
| | CVVVDVSHEDPEVKFNWYVDGVEVHNAKTKPR | |
| | EEQYNSTYRVVSVLTVLHQDWLNGKEYKCKVS | |
| | NKALPAPIEKTISKAKGQPREPQVYTLPPSREEMT | |
| | KNQVSLTCLVKGFYPSDIAVEWESNGQPENNYK | |
| | TTPPVLDSDGSFFLYSKLTVDKSRWQQGNVFSCS | |
| | VLHEALHSHYTQKSLSLSPGK | |
| Flu1_MLNS + GAALIE | | |
| SEQ ID NO: 14 | QVQLQESGPGLVKPSQTLSLTCAISGDSVSSNNAV | Heavy chain |
| | WNWIRQSPSRGLEWLGRTYYRSKWYNDYAESV | |
| | KSRITVNPDTSKNQFSLHLKSVTPEDTAVFYCVRS | |
| | GHITVFGVNVDAFDMWGQGTMVTVSSASTKGPS | |
| | VFPLAPSSKSTSGGTAALGCLVKDYFPEPVTVSW | |
| | NSGALTSGVHTFPAVLQSSGLYSLSSVVTVPSSSL | |
| | GTQTYICNVNHKPSNTKVDKRVEPKSCDKTHTCP | |
| | PCPAPELLAGPSVFLFPPKPKDTLMISRTPEVTCV | |
| | VVDVSHEDPEVKFNWYVDGVEVHNAKTKPREE | |
| | QYNSTYRVVSVLTVLHQDWLNGKEYKCKVSNK | |
| | ALPLPEEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVLH | |
| | EALHSHYTQKSLSLSPGK | |
| Flu1_ALIE | | |
| SEQ ID NO: 15 | QVQLQESGPGLVKPSQTLSLTCAISGDSVSSNNAV | Heavy chain |
| | WNWIRQSPSRGLEWLGRTYYRSKWYNDYAESV | |
| | KSRITVNPDTSKNQFSLHLKSVTPEDTAVFYCVRS | |
| | GHITVFGVNVDAFDMWGQGTMVTVSSASTKGPS | |
| | VFPLAPSSKSTSGGTAALGCLVKDYFPEPVTVSW | |
| | NSGALTSGVHTFPAVLQSSGLYSLSSVVTVPSSSL | |
| | GTQTYICNVNHKPSNTKVDKRVEPKSCDKTHTCP | |
| | PCPAPELLGGPSVFLFPPKPKDTLMISRTPEVTCV | |
| | VVDVSHEDPEVKFNWYVDGVEVHNAKTKPREE | |
| | QYNSTYRVVSVLTVLHQDWLNGKEYKCKVSNK | |
| | ALPLPEEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVM | |
| | HEALHNHYTQKSLSLSPGK | |
| Flu1_MLNS + GRLR | | |
| SEQ ID NO: 16 | QVQLQESGPGLVKPSQTLSLTCAISGDSVSSNNAV | Heavy chain |
| | WNWIRQSPSRGLEWLGRTYYRSKWYNDYAESV | |
| | KSRITVNPDTSKNQFSLHLKSVTPEDTAVFYCVRS | |
| | GHITVFGVNVDAFDMWGQGTMVTVSSASTKGPS | |
| | VFPLAPSSKSTSGGTAALGCLVKDYFPEPVTVSW | |
| | NSGALTSGVHTFPAVLQSSGLYSLSSVVTVPSSSL | |
| | GTQTYICNVNHKPSNTKVDKRVEPKSCDKTHTCP | |
| | PCPAPELLRGPSVFLFPPKPKDTLMISRTPEVTCVV | |
| | VDVSHEDPEVKFNWYVDGVEVHNAKTKPREEQ | |
| | YNSTYRVVSVLTVLHQDWLNGKEYKCKVSNKA | |
| | RPAPIEKTISKAKGQPREPQVYTLPPSREEMTKNQ | |
| | VSLTCLVKGFYPSDIAVEWESNGQPENNYKTTPP | |
| | VLDSDGSFFLYSKLTVDKSRWQQGNVFSCSVLHE | |
| | ALHSHYTQKSLSLSPGK | |
| Flu1_V11 | | |
| SEQ ID NO. 17 | QVQLQESGPGLVKPSQTLSLTCAISGDSVSSNNAV | Heavy chain |
| | WNWIRQSPSRGLEWLGRTYYRSKWYNDYAESV | |
| | KSRITVNPDTSKNQFSLHLKSVTPEDTAVFYCVRS | |
| | GHITVFGVNVDAFDMWGQGTMVTVSSASTKGPS | |
| | VFPLAPSSKSTSGGTAALGCLVKDYFPEPVTVSW | |
| | NSGALTSGVHTFPAVLQSSGLYSLSSVVTVPSSSL | |
| | GTQTYICNVNHKPSNTKVDKRVEPKSCDKTHTCP | |
| | PCPAPELLGDDSVFLFPPKPKDTLMISRTPEVTCV | |
| | VVDVSDEDGEVKFNWYVDGVEVHNAKTKPREE | |
| | QYNSTYRVVSVLTVLHQDWLNGKEYKCKVSNK | |
| | ALPRPIEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVM | |
| | HEALHNHYTQKSLSLSPGK | |
| Flu1_GA | | |
| SEQ ID NO: 18 | QVQLQESGPGLVKPSQTLSLTCAISGDSVSSNNAV | Heavy chain |
| | WNWIRQSPSRGLEWLGRTYYRSKWYNDYAESV | |
| | KSRITVNPDTSKNQFSLHLKSVTPEDTAVFYCVRS | |
| | GHITVFGVNVDAFDMWGQGTMVTVSSASTKGPS | |
| | VFPLAPSSKSTSGGTAALGCLVKDYFPEPVTVSW | |
| | NSGALTSGVHTFPAVLQSSGLYSLSSVVTVPSSSL | |
| | GTQTYICNVNHKPSNTKVDKRVEPKSCDKTHTCP | |
| | PCPAPELLAGPSVFLFPPKPKDTLMISRTPEVTCV | |
| | VVDVSHEDPEVKFNWYVDGVEVHNAKTKPREE | |
| | QYNSTYRVVSVLTVLHQDWLNGKEYKCKVSNK | |
| | ALPAPIEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVM | |
| | HEALHNHYTQKSLSLSPGK | |
| Flu1_MLNS + GA | | |
| SEQ ID NO. 19 | QVQLQESGPGLVKPSQTLSLTCAISGDSVSSNNAV | Heavy chain |
| | WNWIRQSPSRGLEWLGRTYYRSKWYNDYAESV | |
| | KSRITVNPDTSKNQFSLHLKSVTPEDTAVFYCVRS | |
| | GHITVFGVNVDAFDMWGQGTMVTVSSASTKGPS | |
| | VFPLAPSSKSTSGGTAALGCLVKDYFPEPVTVSW | |
| | NSGALTSGVHTFPAVLQSSGLYSLSSVVTVPSSSL | |
| | GTQTYICNVNHKPSNTKVDKRVEPKSCDKTHTCP | |
| | PCPAPELLAGPSVFLFPPKPKDTLMISRTPEVTCV | |
| | VVDVSHEDPEVKFNWYVDGVEVHNAKTKPREE | |
| | QYNSTYRVVSVLTVLHQDWLNGKEYKCKVSNK | |
| | ALPAPIEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVLH | |
| | EALHSHYTQKSLSLSPGK | |

Nucleic acid sequences

| | | |
|---|---|---|
| Flu1_GAALIE | | |
| SEQ ID NO: 20 | CAGGTACAGCTGCAGGAGTCGGGTCCAGGACT | Heavy chain |
| | GGTGAAGCCCTCGCAGACCCTCTCACTCACCTG | |
| | TGCCATCTCCGGGGACAGTGTCTCTAGCAACAA | |
| | TGCTGTTTGGAACTGGATCAGGCAGTCCCCATC | |
| | GAGAGGCCTTGAGTGGCTGGGAAGGACATACT | |
| | ACAGGTCCAAGTGGTATAATGATTATGCAGAA | |
| | TCTGTGAAAAGTCGAATAACCGTCAATCCAGA | |
| | CACATCCAAGAACCAGTTCTCCCTGCACCTGAA | |
| | GTCTGTGACTCCCGAGGACACGGCTGTGTTTTA | |
| | CTGTGTACGATCTGGCCACATTACGGTTTTTGG | |
| | AGTGAATGTTGACGCTTTTGATATGTGGGGCCA | |
| | AGGGACAATGGTCACCGTCTCTTCAGCGTCGAC | |
| | CAAGGGCCCATCGGTCTTCCCCCTGGCACCCTC | |
| | CTCCAAGAGCACCTCTGGGGGCACAGCGGCCC | |
| | TGGGCTGCCTGGTCAAGGACTACTTCCCCGAAC | |
| | CTGTGACGGTCTCGTGGAACTCAGGCGCCCTGA | |
| | CCAGCGGCGTGCACACCTTCCCGGCTGTCCTAC | |
| | AGTCCTCAGGACTCTACTCCCTCAGCAGCGTGG | |
| | TGACCGTGCCCTCCAGCAGCTTGGGCACCCAGA | |
| | CCTACATCTGCAACGTGAATCACAAGCCCAGC | |
| | AACACCAAGGTGGACAAGAGAGTTGAGCCCAA | |
| | ATCTTGTGACAAAACTCACACATGCCCACCGTG | |
| | CCCAGCACCTGAACTCCTGGCCGGACCGTCAGT | |
| | CTTCCTCTTCCCCCCAAAACCCAAGGACACCCT | |
| | CATGATCTCCCGGACCCCTGAGGTCACATGCGT | |
| | GGTGGTGGACGTGAGCCACGAAGACCCTGAGG | |
| | TCAAGTTCAACTGGTACGTGGACGGCGTGGAG | |
| | GTGCATAATGCCAAGACAAAGCCGCGGGAGGA | |
| | GCAGTACAACAGCACGTACCGTGTGGTCAGCG | |
| | TCCTCACCGTCCTGCACCAGGACTGGCTGAATG | |
| | GCAAGGAGTACAAGTGCAAGGTCTCCAACAAA | |
| | GCCCTCCCACTCCCCGAAGAGAAAACCATCTCC | |
| | AAAGCCAAAGGGCAGCCCCGAGAACCACAGGT | |
| | GTACACCCTGCCCCCATCCCGGGAGGAGATGA | |
| | CCAAGAACCAGGTCAGCCTGACCTGCCTGGTC | |
| | AAAGGCTTCTATCCCAGCGACATCGCCGTGGA | |
| | GTGGGAGAGCAATGGGCAGCCGGAGAACAACT | |
| | ACAAGACCACGCCTCCCGTGCTGGACTCCGAC | |
| | GGCTCCTTCTTCCTCTATAGCAAGCTCACCGTG | |
| | GACAAGAGCAGGTGGCAGCAGGGGAACGTCTT | |
| | CTCATGCTCCGTGATGCATGAGGCTCTGCACAA | |
| | CCACTACACGCAGAAGAGCCTCTCCCTGTCCCC | |
| | GGGTAAATGA | |
| Flu1_MLNS | | |
| SEQ ID NO: 21 | CAGGTACAGCTGCAGGAGTCGGGTCCAGGACT | Heavy chain |
| | GGTGAAGCCCTCGCAGACCCTCTCACTCACCTG | |
| | TGCCATCTCCGGGGACAGTGTCTCTAGCAACAA | |
| | TGCTGTTTGGAACTGGATCAGGCAGTCCCCATC | |
| | GAGAGGCCTTGAGTGGCTGGGAAGGACATACT | |
| | ACAGGTCCAAGTGGTATAATGATTATGCAGAA | |
| | TCTGTGAAAAGTCGAATAACCGTCAATCCAGA | |
| | CACATCCAAGAACCAGTTCTCCCTGCACCTGAA | |
| | GTCTGTGACTCCCGAGGACACGGCTGTGTTTTA | |
| | CTGTGTACGATCTGGCCACATTACGGTTTTTGG | |
| | AGTGAATGTTGACGCTTTTGATATGTGGGGCCA | |
| | AGGGACAATGGTCACCGTCTCTTCAGCGTCGAC | |
| | CAAGGGCCCATCGGTCTTCCCCCTGGCACCCTC | |
| | CTCCAAGAGCACCTCTGGGGGCACAGCGGCCC | |
| | TGGGCTGCCTGGTCAAGGACTACTTCCCCGAAC | |
| | CTGTGACGGTCTCGTGGAACTCAGGCGCCCTGA | |
| | CCAGCGGCGTGCACACCTTCCCGGCTGTCCTAC | |
| | AGTCCTCAGGACTCTACTCCCTCAGCAGCGTGG | |
| | TGACCGTGCCCTCCAGCAGCTTGGGCACCCAGA | |
| | CCTACATCTGCAACGTGAATCACAAGCCCAGC | |
| | AACACCAAGGTGGACAAGAGAGTTGAGCCCAA | |
| | ATCTTGTGACAAAACTCACACATGCCCACCGTG | |
| | CCCAGCACCTGAACTCCTGGGGGGACCGTCAG | |
| | TCTTCCTCTTCCCCCCAAAACCCAAGGACACCC | |
| | TCATGATCTCCCGGACCCCTGAGGTCACATGCG | |
| | TGGTGGTGGACGTGAGCCACGAAGACCCTGAG | |
| | GTCAAGTTCAACTGGTACGTGGACGGCGTGGA | |
| | GGTGCATAATGCCAAGACAAAGCCGCGGGAGG | |
| | AGCAGTACAACAGCACGTACCGTGTGGTCAGC | |
| | GTCCTCACCGTCCTGCACCAGGACTGGCTGAAT | |
| | GGCAAGGAGTACAAGTGCAAGGTCTCCAACAA | |
| | AGCCCTCCCAGCCCCCATCGAGAAAACCATCTC | |
| | CAAAGCCAAAGGGCAGCCCCGAGAACCACAGG | |
| | TGTACACCCTGCCCCCATCCCGGGAGGAGATG | |
| | ACCAAGAACCAGGTCAGCCTGACCTGCCTGGT | |
| | CAAAGGCTTCTATCCCAGCGACATCGCCGTGGA | |
| | GTGGGAGAGCAATGGGCAGCCGGAGAACAACT | |
| | ACAAGACCACGCCTCCCGTGCTGGACTCCGAC | |
| | GGCTCCTTCTTCCTCTATAGCAAGCTCACCGTG | |
| | GACAAGAGCAGGTGGCAGCAGGGGAACGTCTT | |
| | CTCATGCTCCGTGCTGCATGAGGCTCTGCACAG | |
| | CCACTACACGCAGAAGAGCCTCTCCCTGTCCCC | |
| | GGGTAAATGA | |
| Flu1_MLNS + GAALIE | | |
| SEQ ID NO: 22 | CAGGTACAGCTGCAGGAGTCGGGTCCAGGACT | Heavy chain |
| | GGTGAAGCCCTCGCAGACCCTCTCACTCACCTG | |
| | TGCCATCTCCGGGGACAGTGTCTCTAGCAACAA | |
| | TGCTGTTTGGAACTGGATCAGGCAGTCCCCATC | |
| | GAGAGGCCTTGAGTGGCTGGGAAGGACATACT | |
| | ACAGGTCCAAGTGGTATAATGATTATGCAGAA | |
| | TCTGTGAAAAGTCGAATAACCGTCAATCCAGA | |
| | CACATCCAAGAACCAGTTCTCCCTGCACCTGAA | |
| | GTCTGTGACTCCCGAGGACACGGCTGTGTTTTA | |
| | CTGTGTACGATCTGGCCACATTACGGTTTTTGG | |
| | AGTGAATGTTGACGCTTTTGATATGTGGGGCCA | |
| | AGGGACAATGGTCACCGTCTCTTCAGCGTCGAC | |
| | CAAGGGCCCATCGGTCTTCCCCCTGGCACCCTC | |
| | CTCCAAGAGCACCTCTGGGGGCACAGCGGCCC | |
| | TGGGCTGCCTGGTCAAGGACTACTTCCCCGAAC | |
| | CTGTGACGGTCTCGTGGAACTCAGGCGCCCTGA | |
| | CCAGCGGCGTGCACACCTTCCCGGCTGTCCTAC | |
| | AGTCCTCAGGACTCTACTCCCTCAGCAGCGTGG | |
| | TGACCGTGCCCTCCAGCAGCTTGGGCACCCAGA | |
| | CCTACATCTGCAACGTGAATCACAAGCCCAGC | |
| | AACACCAAGGTGGACAAGAGAGTTGAGCCCAA | |
| | ATCTTGTGACAAAACTCACACATGCCCACCGTG | |
| | CCCAGCACCTGAACTCCTGGCCGGACCGTCAGT | |
| | CTTCCTCTTCCCCCCAAAACCCAAGGACACCCT | |
| | CATGATCTCCCGGACCCCTGAGGTCACATGCGT | |
| | GGTGGTGGACGTGAGCCACGAAGACCCTGAGG | |
| | TCAAGTTCAACTGGTACGTGGACGGCGTGGAG | |
| | GTGCATAATGCCAAGACAAAGCCGCGGGAGGA | |
| | GCAGTACAACAGCACGTACCGTGTGGTCAGCG | |
| | TCCTCACCGTCCTGCACCAGGACTGGCTGAATG | |
| | GCAAGGAGTACAAGTGCAAGGTCTCCAACAAA | |
| | GCCCTCCCACTCCCCGAAGAGAAAACCATCTCC | |
| | AAAGCCAAAGGGCAGCCCCGAGAACCACAGGT | |
| | GTACACCCTGCCCCCATCCCGGGAGGAGATGA | |
| | CCAAGAACCAGGTCAGCCTGACCTGCCTGGTC | |
| | AAAGGCTTCTATCCCAGCGACATCGCCGTGGA | |
| | GTGGGAGAGCAATGGGCAGCCGGAGAACAACT | |
| | ACAAGACCACGCCTCCCGTGCTGGACTCCGAC | |
| | GGCTCCTTCTTCCTCTATAGCAAGCTCACCGTG | |
| | GACAAGAGCAGGTGGCAGCAGGGGAACGTCTT | |
| | CTCATGCTCCGTGCTGCATGAGGCTCTGCACAG | |
| | CCACTACACGCAGAAGAGCCTCTCCCTGTCCCC | |
| | GGGTAAATGA | |
| Flu1_ALIE | | |
| SEQ ID NO: 23 | CAGGTACAGCTGCAGGAGTCGGGTCCAGGACT | Heavy chain |
| | GGTGAAGCCCTCGCAGACCCTCTCACTCACCTG | |
| | TGCCATCTCCGGGGACAGTGTCTCTAGCAACAA | |
| | TGCTGTTTGGAACTGGATCAGGCAGTCCCCATC | |
| | GAGAGGCCTTGAGTGGCTGGGAAGGACATACT | |
| | ACAGGTCCAAGTGGTATAATGATTATGCAGAA | |
| | TCTGTGAAAAGTCGAATAACCGTCAATCCAGA | |
| | CACATCCAAGAACCAGTTCTCCCTGCACCTGAA | |
| | GTCTGTGACTCCCGAGGACACGGCTGTGTTTTA | |
| | CTGTGTACGATCTGGCCACATTACGGTTTTTGG | |
| | AGTGAATGTTGACGCTTTTGATATGTGGGGCCA | |
| | AGGGACAATGGTCACCGTCTCTTCAGCGTCGAC | |
| | CAAGGGCCCATCGGTCTTCCCCCTGGCACCCTC | |
| | CTCCAAGAGCACCTCTGGGGGCACAGCGGCCC | |
| | TGGGCTGCCTGGTCAAGGACTACTTCCCCGAAC | |
| | CTGTGACGGTCTCGTGGAACTCAGGCGCCCTGA | |
| | CCAGCGGCGTGCACACCTTCCCGGCTGTCCTAC | |
| | AGTCCTCAGGACTCTACTCCCTCAGCAGCGTGG | |
| | TGACCGTGCCCTCCAGCAGCTTGGGCACCCAGA | |
| | CCTACATCTGCAACGTGAATCACAAGCCCAGC | |
| | AACACCAAGGTGGACAAGAGAGTTGAGCCCAA | |
| | ATCTTGTGACAAAACTCACACATGCCCACCGTG | |
| | CCCAGCACCTGAACTCCTGGGGGGACCGTCAG | |
| | TCTTCCTCTTCCCCCCAAAACCCAAGGACACCC | |
| | TCATGATCTCCCGGACCCCTGAGGTCACATGCG | |
| | TGGTGGTGGACGTGAGCCACGAAGACCCTGAG | |
| | GTCAAGTTCAACTGGTACGTGGACGGCGTGGA | |
| | GGTGCATAATGCCAAGACAAAGCCGCGGGAGG | |
| | AGCAGTACAACAGCACGTACCGTGTGGTCAGC | |
| | GTCCTCACCGTCCTGCACCAGGACTGGCTGAAT | |
| | GGCAAGGAGTACAAGTGCAAGGTCTCCAACAA | |
| | AGCCCTCCCACTCCCCGAGGAGAAAACCATCTC | |
| | CAAAGCCAAAGGGCAGCCCCGAGAACCACAGG | |
| | TGTACACCCTGCCCCCATCCCGGGAGGAGATG | |
| | ACCAAGAACCAGGTCAGCCTGACCTGCCTGGT | |
| | CAAAGGCTTCTATCCCAGCGACATCGCCGTGGA | |
| | GTGGGAGAGCAATGGGCAGCCGGAGAACAACT | |
| | ACAAGACCACGCCTCCCGTGCTGGACTCCGAC | |
| | GGCTCCTTCTTCCTCTATAGCAAGCTCACCGTG | |
| | GACAAGAGCAGGTGGCAGCAGGGGAACGTCTT | |
| | CTCATGCTCCGTGATGCATGAGGCTCTGCACAA | |
| | CCACTACACGCAGAAGAGCCTCTCCCTGTCCCC | |
| | GGGTAAATGA | |
| Flu1_GA | | |
| SEQ ID NO: 24 | CAGGTACAGCTGCAGGAGTCGGGTCCAGGACT | Heavy chain |
| | GGTGAAGCCCTCGCAGACCCTCTCACTCACCTG | |
| | TGCCATCTCCGGGGACAGTGTCTCTAGCAACAA | |
| | TGCTGTTTGGAACTGGATCAGGCAGTCCCCATC | |
| | GAGAGGCCTTGAGTGGCTGGGAAGGACATACT | |
| | ACAGGTCCAAGTGGTATAATGATTATGCAGAA | |
| | TCTGTGAAAAGTCGAATAACCGTCAATCCAGA | |
| | CACATCCAAGAACCAGTTCTCCCTGCACCTGAA | |
| | GTCTGTGACTCCCGAGGACACGGCTGTGTTTTA | |
| | CTGTGTACGATCTGGCCACATTACGGTTTTTGG | |
| | AGTGAATGTTGACGCTTTTGATATGTGGGGCCA | |
| | AGGGACAATGGTCACCGTCTCTTCAGCGTCGAC | |
| | CAAGGGCCCATCGGTCTTCCCCCTGGCACCCTC | |
| | CTCCAAGAGCACCTCTGGGGGCACAGCGGCCC | |
| | TGGGCTGCCTGGTCAAGGACTACTTCCCCGAAC | |
| | CTGTGACGGTCTCGTGGAACTCAGGCGCCCTGA | |
| | CCAGCGGCGTGCACACCTTCCCGGCTGTCCTAC | |
| | AGTCCTCAGGACTCTACTCCCTCAGCAGCGTGG | |
| | TGACCGTGCCCTCCAGCAGCTTGGGCACCCAGA | |
| | CCTACATCTGCAACGTGAATCACAAGCCCAGC | |
| | AACACCAAGGTGGACAAGAGAGTTGAGCCCAA | |
| | ATCTTGTGACAAAACTCACACATGCCCACCGTG | |
| | CCCAGCACCTGAACTCCTGGCGGGACCGTCAGT | |
| | CTTCCTCTTCCCCCCAAAACCCAAGGACACCCT | |
| | CATGATCTCCCGGACCCCTGAGGTCACATGCGT | |
| | GGTGGTGGACGTGAGCCACGAAGACCCTGAGG | |
| | TCAAGTTCAACTGGTACGTGGACGGCGTGGAG | |
| | GTGCATAATGCCAAGACAAAGCCGCGGGAGGA | |
| | GCAGTACAACAGCACGTACCGTGTGGTCAGCG | |
| | TCCTCACCGTCCTGCACCAGGACTGGCTGAATG | |
| | GCAAGGAGTACAAGTGCAAGGTCTCCAACAAA | |
| | GCCCTCCCAGCCCCCATCGAGAAAACCATCTCC | |
| | AAAGCCAAAGGGCAGCCCCGAGAACCACAGGT | |
| | GTACACCCTGCCCCCATCCCGGGAGGAGATGA | |
| | CCAAGAACCAGGTCAGCCTGACCTGCCTGGTC | |
| | AAAGGCTTCTATCCCAGCGACATCGCCGTGGA | |
| | GTGGGAGAGCAATGGGCAGCCGGAGAACAACT | |
| | ACAAGACCACGCCTCCCGTGCTGGACTCCGAC | |
| | GGCTCCTTCTTCCTCTATAGCAAGCTCACCGTG | |
| | GACAAGAGCAGGTGGCAGCAGGGGAACGTCTT | |
| | CTCATGCTCCGTGATGCATGAGGCTCTGCACAA | |
| | CCACTACACGCAGAAGAGCCTCTCCCTGTCCCC | |
| | GGGTAAATGA | |
| Flu1_MLNS + GA | | |
| SEQ ID NO: 25 | CAGGTACAGCTGCAGGAGTCGGGTCCAGGACT | Heavy chain |
| | GGTGAAGCCCTCGCAGACCCTCTCACTCACCTG | |
| | TGCCATCTCCGGGGACAGTGTCTCTAGCAACAA | |
| | TGCTGTTTGGAACTGGATCAGGCAGTCCCCATC | |
| | GAGAGGCCTTGAGTGGCTGGGAAGGACATACT | |
| | ACAGGTCCAAGTGGTATAATGATTATGCAGAA | |
| | TCTGTGAAAAGTCGAATAACCGTCAATCCAGA | |
| | CACATCCAAGAACCAGTTCTCCCTGCACCTGAA | |
| | GTCTGTGACTCCCGAGGACACGGCTGTGTTTTA | |
| | CTGTGTACGATCTGGCCACATTACGGTTTTTGG | |
| | AGTGAATGTTGACGCTTTTGATATGTGGGGCCA | |
| | AGGGACAATGGTCACCGTCTCTTCAGCGTCGAC | |
| | CAAGGGCCCATCGGTCTTCCCCCTGGCACCCTC | |
| | CTCCAAGAGCACCTCTGGGGGCACAGCGGCCC | |
| | TGGGCTGCCTGGTCAAGGACTACTTCCCCGAAC | |
| | CTGTGACGGTCTCGTGGAACTCAGGCGCCCTGA | |
| | CCAGCGGCGTGCACACCTTCCCGGCTGTCCTAC | |
| | AGTCCTCAGGACTCTACTCCCTCAGCAGCGTGG | |
| | TGACCGTGCCCTCCAGCAGCTTGGGCACCCAGA | |
| | CCTACATCTGCAACGTGAATCACAAGCCCAGC | |
| | AACACCAAGGTGGACAAGAGAGTTGAGCCCAA | |
| | ATCTTGTGACAAAACTCACACATGCCCACCGTG | |
| | CCCAGCACCTGAACTCCTGGCGGGACCGTCAGT | |
| | CTTCCTCTTCCCCCCAAAACCCAAGGACACCCT | |
| | CATGATCTCCCGGACCCCTGAGGTCACATGCGT | |
| | GGTGGTGGACGTGAGCCACGAAGACCCTGAGG | |
| | TCAAGTTCAACTGGTACGTGGACGGCGTGGAG | |
| | GTGCATAATGCCAAGACAAAGCCGCGGGAGGA | |
| | GCAGTACAACAGCACGTACCGTGTGGTCAGCG | |
| | TCCTCACCGTCCTGCACCAGGACTGGCTGAATG | |
| | GCAAGGAGTACAAGTGCAAGGTCTCCAACAAA | |
| | GCCCTCCCAGCCCCCATCGAGAAAACCATCTCC | |
| | AAAGCCAAAGGGCAGCCCCGAGAACCACAGGT | |
| | GTACACCCTGCCCCCATCCCGGGAGGAGATGA | |
| | CCAAGAACCAGGTCAGCCTGACCTGCCTGGTC | |
| | AAAGGCTTCTATCCCAGCGACATCGCCGTGGA | |
| | GTGGGAGAGCAATGGGCAGCCGGAGAACAACT | |
| | ACAAGACCACGCCTCCCGTGCTGGACTCCGAC | |
| | GGCTCCTTCTTCCTCTATAGCAAGCTCACCGTG | |
| | GACAAGAGCAGGTGGCAGCAGGGGAACGTCTT | |
| | CTCATGCTCCGTGCTGCATGAGGCTCTGCACAG | |
| | CCACTACACGCAGAAGAGCCTCTCCCTGTCCCC | |
| | GGGTAAATGA | |
| FI6v3 | | |
| SEQ ID NO: 26 | GFTFSTYA | CDRH1 |
| SEQ ID NO: 27 | ISYDANYK | CDRH2 |
| SEQ ID NO: 28 | AKDSQLRSLLYFEWLSQGYFDY | CDRH3 |
| SEQ ID NO: 29 | SSQSVTFNYKNY | CDRL1 |
| SEQ ID NO: 30 | WAS | CDRL2 |
| SEQ ID NO. 31 | QQHYRTPPT | CDRL3 |
| SEQ ID NO: 32 | QVQLVESGGGVVQPGRSLRLSCAASGFTFSTYAM | VH |
| | HWVRQAPGKGLEWVAVISYDANYKYYADSVKG | |
| | RFTISRDNSKNTLYLQMNSLRAEDTAVYYCAKDS | |
| | QLRSLLYFEWLSQGYFDYWGQGTLVTVSR | |
| SEQ ID NO. 33 | YGDIVMTQSPDSLAVSLGERATINCKSSQSVTFNY | VL |
| | KNYLAWYQQKPGQPPKLLIYWASTRESGVPDRF | |
| | SGSGSGTDFTLTISSLQAEDVAVYYCQQHYRTPPT | |
| | FGQGTKVDSR | |
| SEQ ID NO: 34 | MEFGLSWVFLVALLRGVQCQVQLVESGGGVVQP | Heavy chain |
| | GRSLRLSCAASGFTFSTYAMHWVRQAPGKGLEW | |
| | VAVISYDANYKYYADSVKGRFTISRDNSKNTLYL | |
| | QMNSLRAEDTAVYYCAKDSQLRSLLYFEWLSQG | |
| | YFDYWGQGTLVTVSRASTKGPSVFPLAPSSKSTS | |
| | GGTAALGCLVKDYFPEPVTVSWNSGALTSGVHT | |
| | FPAVLQSSGLYSLSSVVTVPSSSLGTQTYICNVNH | |
| | KPSNTKVDKRVEPKSCDKTHTCPPCPAPELLGGP | |
| | SVFLFPPKPKDTLMISRTPEVTCVVVDVSHEDPEV | |
| | KFNWYVDGVEVHNAKTKPREEQYNSTYRVVSV | |
| | LTVLHQDWLNGKEYKCKVSNKALPAPIEKTISKA | |
| | KGQPREPQVYTLPPSRDELTKNQVSLTCLVKGFY | |
| | PSDIAVEWESNGQPENNYKTTPPVLDSDGSFFLYS | |
| | KLTVDKSRWQQGNVFSCSVMHEALHNHYTQKS | |
| | LSLSPGK | |
| SEQ ID NO. 35 | MVLQTQVFISLLLWISGAYGDIVMTQSPDSLAVS | Light chain |
| | LGERATINCKSSQSVTFNYKNYLAWYQQKPGQPP | |
| | KLLIYWASTRESGVPDRFSGSGSGTDFTLTISSLQ | |
| | AEDVAVYYCQQHYRTPPTFGQGTKVDSRRTVAA | |
| | PSVFIFPPSDEQLKSGTASVVCLLNNFYPREAKVQ | |
| | WKVDNALQSGNSQESVTEQDSKDSTYSLSSTLTL | |
| | SKADYEKHKVYACEVTHQGLSSPVTKSFNRGEC | |
| 3C05 | | |
| SEQ ID NO: 36 | GGSFGGYY | CDRH1 |
| SEQ ID NO: 37 | INHSGST | CDRH2 |
| SEQ ID NO: 38 | ARGRGGYATYYYYYYVDV | CDRH3 |
| SEQ ID NO: 39 | QSVSSY | CDRL1 |
| SEQ ID NO: 40 | DAS | CDRL2 |
| SEQ ID NO. 41 | QQRSNWLT | CDRL3 |
| SEQ ID NO: 42 | QVQLQQWGAGLLKPSETLSLTCAVYGGSFGGYY | VH |
| | WNWIRQPPGKGLEWIGEINHSGSTNYNPSLKSRV | |
| | TLSVDTSKNQVSLNVSSVTAADTAVYYCARGRG | |
| | GYATYYYYYYVDVWGKGTTVTVSS | |
| SEQ ID NO: 43 | EIVLTQSPATLSLSPGERATLSCRASQSVSSYLAW | VL |
| | YQQKPGQAPRLLIYDASKRATGIPARFSGSGSGTD | |
| | FTLTISSLEPEDFAVYYCQQRSNWLTFGGGTKVE | |
| | LE | |
| SEQ ID NO: 44 | MGWSCIILFLVATATGVHSQVQLQQWGAGLLKP | Heavy chain |
| | SETLSLTCAVYGGSFGGYYWNWIRQPPGKGLEWI | |
| | GEINHSGSTNYNPSLKSRVTLSVDTSKNQVSLNVS | |
| | SVTAADTAVYYCARGRGGYATYYYYYYVDVW | |
| | GKGTTVTVSSSTKGPSVFPLAPSSKSTSGGTAALG | |
| | CLVKDYFPEPVTVSWNSGALTSGVHTFPAVLQSS | |
| | GLYSLSSVVTVPSSSLGTQTYICNVNHKPSNTKVD | |
| | KRVEPKSCDKTHTCPPCPAPELLGGPSVFLFPPKP | |
| | KDTLMISRTPEVTCVVVDVSHEDPEVKFNWYVD | |
| | GVEVHNAKTKPREEQYNSTYRVVSVLTVLHQDW | |
| | LNGKEYKCKVSNKALPAPIEKTISKAKGQPREPQ | |
| | VYTLPPSRDELTKNQVSLTCLVKGFYPSDIAVEW | |
| | ESNGQPENNYKTTPPVLDSDGSFFLYSKLTVDKS | |
| | RWQQGNVFSCSVMHEALHNHYTQKSLSLSPGK | |
| SEQ ID NO: 45 | MGWSCIILFLVATATGHSEIVLTQSPATLSLSPGER | Light chain |
| | ATLSCRASQSVSSYLAWYQQKPGQAPRLLIYDAS | |
| | KRATGIPARFSGSGSGTDFTLTISSLEPEDFAVYYC | |
| | QQRSNWLTFGGGTKVELERTVAAPSVFIFPPSDE | |
| | QLKSGTASVVCLLNNFYPREAKVQWKVDNALQS | |
| | GNSQESVTEQDSKDSTYSLSSTLTLSKADYEKHK | |
| | VYACEVTHQGLSSPVTKSFNRGEC | |
| TCN032 | | |
| SEQ ID NO: 46 | GSSISNYY | CDRH1 |
| SEQ ID NO: 47 | IYYGGNT | CDRH2 |
| SEQ ID NO: 48 | ARASCSGGYCILDY | CDRH3 |
| SEQ ID NO: 49 | QNIYKY | CDRL1 |
| SEQ ID NO: 50 | AAS | CDRL2 |
| SEQ ID NO. 51 | QQSYSPPLT | CDRL3 |
| SEQ ID NO: 52 | QVQLQESGPGLVKPSETLSLTCTVSGSSISNYYWS | VH |
| | WIRQSPGKGLEWIGFIYYGGNTKYNPSLKSRVTIS | |
| | QDTSKSQVSLTMSSVTAAESAVYFCARASCSGGY | |
| | CILDYWGQGTLVTVSS | |
| SEQ ID NO: 53 | DIQMTQSPSSLSASVGDRVTITCRASQNIYKYLN | VL |
| | WYQQRPGKAPKGLISAASGLQSGVPSRFSGSGSG | |
| | TDFTLTITSLQPEDFATYYCQQSYSPPLTFGGGTR | |
| | VEIK | |
| SEQ ID NO: 54 | MGWSCIILFLVATATGAHSQVQLQESGPGLVKPS | Heavy chain |
| | ETLSLTCTVSGSSISNYYWSWIRQSPGKGLEWIGF | |
| | IYYGGNTKYNPSLKSRVTISQDTSKSQVSLTMSSV | |
| | TAAESAVYFCARASCSGGYCILDYWGQGTLVTV | |
| | SSASTKGPSVFPLAPSSKSTSGGTAALGCLVKDYF | |
| | PEPVTVSWNSGALTSGVHTFPAVLQSSGLYSLSS | |
| | VVTVPSSSLGTQTYICNVNHKPSNTKVDKKVEPK | |
| | SCDKTHTCPPCPAPELLGGPSVFLFPPKPKDTLMIS | |
| | RTPEVTCVVVDVSHEDPEVKFNWYVDGVEVHN | |
| | AKTKPREEQYNSTYRVVSVLTVLHQDWLNGKEY | |
| | KCKVSNKALPAPIEKTISKAKGQPREPQVYTLPPS | |
| | REEMTKNQVSLTCLVKGFYPSDIAVEWESNGQPE | |
| | NNYKTTPPVLDSDGSFFLYSKLTVDKSRWQQGN | |
| | VFSCSVMHEALHNHYTQKSLSLSPGK | |
| SEQ ID NO. 55 | MGWSCIILFLVATATGVHDIQMTQSPSSLSASVG | Light chain |
| | DRVTITCRASQNIYKYLNWYQQRPGKAPKGLISA | |
| | ASGLQSGVPSRFSGSGSGTDFTLTITSLQPEDFATY | |
| | YCQQSYSPPLTFGGGTRVEIKRTVDAPSVFIFPPSD | |
| | EQLKSGTASVVCLLNNFYPREAKVQWKVDNALQ | |
| | SGNSQESVTEQDSKDSTYSLSSTLTLSKADYEKH | |
| | KVYACEVTHQGLSSPVTKSFNRGEC | |
| 14C2 | | |
| SEQ ID NO: 56 | GYIFTDYA | CDRH1 |
| SEQ ID NO: 57 | ISTYTGKT | CDRH2 |
| SEQ ID NO: 58 | ARRGDYDAWFAY | CDRH3 |
| SEQ ID NO: 59 | QRLLYSSDQKNY | CDRL1 |
| SEQ ID NO: 60 | WAS | CDRL2 |
| SEQ ID NO: 61 | QQYYTYPLT | CDRL3 |
| SEQ ID NO. 62 | QVQLQQSGPEVVRPGVSVKISCKGSGYIFTDYAM | VH |
| | HWVKQSHAKSLEWIGVISTYTGKTNYSQKFKGK | |
| | ATMTVDKSSSTAYMELARLTSEDSSVYYCARRG | |
| | DYDAWFAYWGQGTLVTVSS | |
| SEQ ID NO: 63 | DIVMSQSPSSLAVSVGEKVSMTCKSSQRLLYSSD | VL |
| | QKNYLAWYQQKPGQSPKVLIYWASTRVSGVPDR | |
| | FTGSESGTDFTLTISSVKAEDLAVYYCQQYYTYPL | |
| | TFGAGTKLELK | |
| SEQ ID NO: 64 | MGWSCIILFLVATATGAHSQVQLQQSGPEVVRPG | Heavy chain |
| | VSVKISCKGSGYIFTDYAMTIWVKQSHAKSLEWI | |
| | GVISTYTGKTNYSQKFKGKATMTVDKSSSTAYM | |
| | ELARLTSEDSSVYYCARRGDYDAWFAWGQGT | |
| | LVTVSSASTKGPSVFPLAPSSKSTSGGTAALGCLV | |
| | KDYFPEPVTVSWNSGALTSGVHTFPAVLQSSGLY | |
| | SLSSVVTVPSSSLGTQTYICNVNHKPSNTKVDKK | |
| | VEPKSCDKTHTCPPCPAPELLGGPSVFLFPPKPKD | |
| | TLMISRTPEVTCVVVDVSHEDPEVKFNWYVDGV | |
| | EVHNAKTKPREEQYNSTYRVVSVLTVLHQDWLN | |
| | GKEYKCKVSNKALPAPIEKTISKAKGQPREPQVY | |
| | TLPPSREEMTKNQVSLTCLVKGFYPSDIAVEWES | |
| | NGQPENNYKTTPPVLDSDGSFFLYSKLTVDKSRW | |
| | QQGNVFSCSVMHEALHNHYTQKSLSLSPGK | |
| SEQ ID NO: 65 | MGWSCIILFLVATATGVHDIVMSQSPSSLAVSVG | Light chain |
| | EKVSMTCKSSQRLLYSSDQKNYLAWYQQKPGQS | |
| | PKVLIYWASTRVSGVPDRFTGSESGTDFTLTISSV | |
| | KAEDLAVYYCQQYYTYPLTFGAGTKLELKRTVD | |
| | APSVFIFPPSDEQLKSGTASVVCLLNNFYPREAKV | |
| | QWKVDNALQSGNSQESVTEQDSKDSTYSLSSTLT | |
| | LSKADYEKHKVYACEVTHQGLSSPVTKSFNRGE | |
| | C | |
| FI6v3 - WT | | |
| SEQ ID NO: 66 | MNSLRAEDTAVYYCAKDSQLRSLLYFEWLSQGYFD | Heavy Chain |
| | YWGQGTLVTVSRASTKGPSVFPLAPSSKSTSGGTAAL | |
| | GCLVKDYFPEPVTVSWNSGALTSGVHTFPAVLQSSG | |
| | LYSLSSVVTVPSSSLGTQTYICNVNHKPSNTKVDKRV | |
| | EPKSCDKTHTCPPCPAPELLGGPSVFLFPPKPKDTLMI | |
| | SRTPEVTCVVVDVSHEDPEVKFNWYVDGVEVHNAK | |
| | TKPREEQYNSTYRVVSVLTVLHQDWLNGKEYKCKV | |
| | SNKALPAPIEKTISKAKGQPREPQVYTLPPSRDELTKN | |
| | QVSLTCLVKGFYPSDIAVEWESNGQPENNYKTTPPVL | |
| | DSDGSFFLYSKLTVDKSRWQQGNVFSCSVMHEALH | |
| | NHYTQKSLSLSPGK | |
| FI6v3 - GRLR | | |
| SEQ ID NO: 67 | MNSLRAEDTAVYYCAKDSQLRSLLYFEWLSQGYFD | Heavy Chain |
| | YWGQGTLVTVSRASTKGPSVFPLAPSSKSTSGGTAAL | |
| | GCLVKDYFPEPVTVSWNSGALTSGVHTFPAVLQSSG | |
| | LYSLSSVVTVPSSSLGTQTYICNVNHKPSNTKVDKRV | |
| | EPKSCDKTHTCPPCPAPELLRGPSVFLFPPKPKDTLMI | |
| | SRTPEVTCVVVDVSHEDPEVKFNWYVDGVEVHNAK | |
| | TKPREEQYNSTYRVVSVLTVLHQDWLNGKEYKCKV | |
| | SNKARPAPIEKTISKAKGQPREPQVYTLPPSRDELTKN | |
| | QVSLTCLVKGFYPSDIAVEWESNGQPENNYKTTPPVL | |
| | DSDGSFFLYSKLTVDKSRWQQGNVFSCSVMHEALH | |
| | NHYTQKSLSLSPGK | |
| FI6v3 - GA | | |
| SEQ ID NO: 68 | MNSLRAEDTAVYYCAKDSQLRSLLYFEWLSQGYFD | Heavy Chain |
| | YWGQGTLVTVSRASTKGPSVFPLAPSSKSTSGGTAAL | |
| | GCLVKDYFPEPVTVSWNSGALTSGVHTFPAVLQSSG | |
| | LYSLSSVVTVPSSSLGTQTYICNVNHKPSNTKVDKRV | |
| | EPKSCDKTHTCPPCPAPELLAGPSVFLFPPKPKDTLMI | |
| | SRTPEVTCVVVDVSHEDPEVKFNWYVDGVEVHNAK | |
| | TKPREEQYNSTYRVVSVLTVLHQDWLNGKEYKCKV | |
| | SNKALPAPIEKTISKAKGQPREPQVYTLPPSRDELTKN | |
| | QVSLTCLVKGFYPSDIAVEWESNGQPENNYKTTPPVL | |
| | DSDGSFFLYSKLTVDKSRWQQGNVFSCSVMHEALH | |
| | NHYTQKSLSLSPGK | |
| FI6v3 - ALIE | | |
| SEQ ID NO: 69 | MNSLRAEDTAVYYCAKDSQLRSLLYFEWLSQGYFD | Heavy Chain |
| | YWGQGTLVTVSRASTKGPSVFPLAPSSKSTSGGTAAL | |
| | GCLVKDYFPEPVTVSWNSGALTSGVHTFPAVLQSSG | |
| | LYSLSSVVTVPSSSLGTQTYICNVNHKPSNTKVDKRV | |
| | EPKSCDKTHTCPPCPAPELLGGPSVFLFPPKPKDTLMI | |
| | SRTPEVTCVVVDVSHEDPEVKFNWYVDGVEVHNAK | |
| | TKPREEQYNSTYRVVSVLTVLHQDWLNGKEYKCKV | |
| | SNKALPLPEEKTISKAKGQPREPQVYTLPPSRDELTKN | |
| | QVSLTCLVKGFYPSDIAVEWESNGQPENNYKTTPPVL | |
| | DSDGSFFLYSKLTVDKSRWQQGNVFSCSVMHEALH | |
| | NHYTQKSLSLSPGK | |
| FI6v3 - GAALIE | | |
| SEQ ID NO: 70 | MNSLRAEDTAVYYCAKDSQLRSLLYFEWLSQGYFD | Heavy Chain |
| | YWGQGTLVTVSRASTKGPSVFPLAPSSKSTSGGTAAL | |
| | GCLVKDYFPEPVTVSWNSGALTSGVHTFPAVLQSSG | |
| | LYSLSSVVTVPSSSLGTQTYICNVNHKPSNTKVDKRV | |
| | EPKSCDKTHTCPPCPAPELLAGPSVFLFPPKPKDTLMI | |
| | SRTPEVTCVVVDVSHEDPEVKFNWYVDGVEVHNAK | |
| | TKPREEQYNSTYRVVSVLTVLHQDWLNGKEYKCKV | |
| | SNKALPLPEEKTISKAKGQPREPQVYTLPPSRDELTKN | |
| | QVSLTCLVKGFYPSDIAVEWESNGQPENNYKTTPPVL | |
| | DSDGSFFLYSKLTVDKSRWQQGNVFSCSVMHEALH | |
| | NHYTQKSLSLSPGK | |
| 3C05 - WT | | |
| SEQ ID NO: 71 | MGWSCIILFLVATATGVHSQVQLQQWGAGLLKPSET | Heavy Chain |
| | LSLTCAVYGGSFGGYYWNWIRQPPGKGLEWIGEINH | |
| | SGSTNYNPSLKSRVTLSVDTSKNQVSLNVSSVTAADT | |
| | AVYYCARGRGGYATYYYYYYVDVWGKGTTVTVSS | |
| | STKGPSVFPLAPSSKSTSGGTAALGCLVKDYFPEPVT | |
| | VSWNSGALTSGVHTFPAVLQSSGLYSLSSVVTVPSSS | |
| | LGTQTYICNVNHKPSNTKVDKRVEPKSCDKTHTCPP | |
| | CPAPELLGGPSVFLFPPKPKDTLMISRTPEVTCVVVD | |
| | VSHEDPEVKFNWYVDGVEVHNAKTKPREEQYNSTY | |
| | RVVSVLTVLHQDWLNGKEYKCKVSNKALPAPIEKTI | |
| | SKAKGQPREPQVYTLPPSRDELTKNQVSLTCLVKGFY | |
| | PSDIAVEWESNGQPENNYKTTPPVLDSDGSFFLYSKL | |
| | TVDKSRWQQGNVFSCSVMHEALHNHYTQKSLSLSP | |
| | GK | |
| 3C05 - GRLR | | |
| SEQ ID NO: 72 | MGWSCIILFLVATATGVHSQVQLQQWGAGLLKPSET | Heavy Chain |
| | LSLTCAVYGGSFGGYYWNWIRQPPGKGLEWIGEINH | |
| | SGSTNYNPSLKSRVTLSVDTSKNQVSLNVSSVTAADT | |
| | AVYYCARGRGGYATYYYYYYVDVWGKGTTVTVSS | |
| | STKGPSVFPLAPSSKSTSGGTAALGCLVKDYFPEPVT | |
| | VSWNSGALTSGVHTFPAVLQSSGLYSLSSVVTVPSSS | |
| | LGTQTYICNVNHKPSNTKVDKRVEPKSCDKTHTCPP | |
| | CPAPELLRGPSVFLFPPKPKDTLMISRTPEVTCVVVDV | |
| | SHEDPEVKFNWYVDGVEVHNAKTKPREEQYNSTYR | |
| | VVSVLTVLHQDWLNGKEYKCKVSNKARPAPIEKTIS | |
| | KAKGQPREPQVYTLPPSRDELTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPG | |
| | K | |
| 3C05 - GA | | |
| SEQ ID NO: 73 | MGWSCIILFLVATATGVHSQVQLQQWGAGLLKPSET | Heavy Chain |
| | LSLTCAVYGGSFGGYYWNWIRQPPGKGLEWIGEINH | |
| | SGSTNYNPSLKSRVTLSVDTSKNQVSLNVSSVTAADT | |
| | AVYYCARGRGGYATYYYYYYVDVWGKGTTVTVSS | |
| | STKGPSVFPLAPSSKSTSGGTAALGCLVKDYFPEPVT | |
| | VSWNSGALTSGVHTFPAVLQSSGLYSLSSVVTVPSSS | |
| | LGTQTYICNVNHKPSNTKVDKRVEPKSCDKTHTCPP | |
| | CPAPELLAGPSVFLFPPKPKDTLMISRTPEVTCVVVD | |
| | VSHEDPEVKFNWYVDGVEVHNAKTKPREEQYNSTY | |
| | RVVSVLTVLHQDWLNGKEYKCKVSNKALPAPIEKTI | |
| | SKAKGQPREPQVYTLPPSRDELTKNQVSLTCLVKGFY | |
| | PSDIAVEWESNGQPENNYKTTPPVLDSDGSFFLYSKL | |
| | TVDKSRWQQGNVFSCSVMHEALHNHYTQKSLSLSP | |
| | GK | |
| 3C05 - ALIE | | |
| SEQ ID NO: 74 | MGWSCIILFLVATATGVHSQVQLQQWGAGLLKPSET | Heavy Chain |
| | LSLTCAVYGGSFGGYYWNWIRQPPGKGLEWIGEINH | |
| | SGSTNYNPSLKSRVTLSVDTSKNQVSLNVSSVTAADT | |
| | AVYYCARGRGGYATYYYYYYVDVWGKGTTVTVSS | |
| | STKGPSVFPLAPSSKSTSGGTAALGCLVKDYFPEPVT | |
| | VSWNSGALTSGVHTFPAVLQSSGLYSLSSVVTVPSSS | |
| | LGTQTYICNVNHKPSNTKVDKRVEPKSCDKTHTCPP | |
| | CPAPELLGGPSVFLFPPKPKDTLMISRTPEVTCVVVD | |
| | VSHEDPEVKFNWYVDGVEVHNAKTKPREEQYNSTY | |
| | RVVSVLTVLHQDWLNGKEYKCKVSNKALPLPEEKTI | |
| | SKAKGQPREPQVYTLPPSRDELTKNQVSLTCLVKGFY | |
| | PSDIAVEWESNGQPENNYKTTPPVLDSDGSFFLYSKL | |
| | TVDKSRWQQGNVFSCSVMHEALHNHYTQKSLSLSP | |
| | GK | |
| 3C05 - GAALIE | | |
| SEQ ID NO: 75 | MGWSCIILFLVATATGVHSQVQLQQWGAGLLKPSET | Heavy Chain |
| | LSLTCAVYGGSFGGYYWNWIRQPPGKGLEWIGEINH | |
| | SGSTNYNPSLKSRVTLSVDTSKNQVSLNVSSVTAADT | |
| | AVYYCARGRGGYATYYYYYYVDVWGKGTTVTVSS | |
| | STKGPSVFPLAPSSKSTSGGTAALGCLVKDYFPEPVT | |
| | VSWNSGALTSGVHTFPAVLQSSGLYSLSSVVTVPSSS | |
| | LGTQTYICNVNHKPSNTKVDKRVEPKSCDKTHTCPP | |
| | CPAPELLAGPSVFLFPPKPKDTLMISRTPEVTCVVVD | |
| | VSHEDPEVKFNWYVDGVEVHNAKTKPREEQYNSTY | |
| | RVVSVLTVLHQDWLNGKEYKCKVSNKALPLPEEKTI | |
| | SKAKGQPREPQVYTLPPSRDELTKNQVSLTCLVKGFY | |
| | PSDIAVEWESNGQPENNYKTTPPVLDSDGSFFLYSKL | |
| | TVDKSRWQQGNVFSCSVMHEALHNHYTQKSLSLSP | |
| | GK | |
| TCN032 - WT | | |
| SEQ ID NO: 76 | MGWSCIILFLVATATGAHSQVQLQESGPGLVKPSETL | Heavy Chain |
| | SLTCTVSGSSISNYYWSWIRQSPGKGLEWIGFIYYGG | |
| | NTKYNPSLKSRVTISQDTSKSQVSLTMSSVTAAESAV | |
| | YFCARASCSGGYCILDWGQGTLVTVSSASTKGPSV | |
| | FPLAPSSKSTSGGTAALGCLVKDYFPEPVTVSWNSGA | |
| | LTSGVHTFPAVLQSSGLYSLSSVVTVPSSSLGTQTYIC | |
| | NVNHKPSNTKVDKKVEPKSCDKTHTCPPCPAPELLG | |
| | GPSVFLFPPKPKDTLMISRTPEVTCVVVDVSHEDPEV | |
| | KFNWYVDGVEVHNAKTKPREEQYNSTYRVVSVLTV | |
| | LHQDWLNGKEYKCKVSNKALPAPIEKTISKAKGQPR | |
| | EPQVYTLPPSREEMTKNQVSLTCLVKGFYPSDIAVEW | |
| | ESNGQPENNYKTTPPVLDSDGSFFLYSKLTVDKSRW | |
| | QQGNVFSCSVMHEALHNHYTQKSLSLSPGK | |
| TCN032 - GRLR | | |
| SEQ ID NO: 77 | MGWSCIILFLVATATGAHSQVQLQESGPGLVKPSETL | Heavy Chain |
| | SLTCTVSGSSISNYYWSWIRQSPGKGLEWIGFIYYGG | |
| | NTKYNPSLKSRVTISQDTSKSQVSLTMSSVTAAESAV | |
| | YFCARASCSGGYCILDWGQGTLVTVSSASTKGPSV | |
| | FPLAPSSKSTSGGTAALGCLVKDYFPEPVTVSWNSGA | |
| | LTSGVHTFPAVLQSSGLYSLSSVVTVPSSSLGTQTYIC | |
| | NVNHKPSNTKVDKKVEPKSCDKTHTCPPCPAPELLR | |
| | GPSVFLFPPKPKDTLMISRTPEVTCVVVDVSHEDPEV | |
| | KFNWYVDGVEVHNAKTKPREEQYNSTYRVVSVLTV | |
| | LHQDWLNGKEYKCKVSNKARPAPIEKTISKAKGQPR | |
| | EPQVYTLPPSREEMTKNQVSLTCLVKGFYPSDIAVEW | |
| | ESNGQPENNYKTTPPVLDSDGSFFLYSKLTVDKSRW | |
| | QQGNVFSCSVMHEALHNHYTQKSLSLSPGK | |
| TCN032 - GA | | |
| SEQ ID NO: 78 | MGWSCIILFLVATATGAHSQVQLQESGPGLVKPSETL | Heavy Chain |
| | SLTCTVSGSSISNYYWSWIRQSPGKGLEWIGFIYYGG | |
| | NTKYNPSLKSRVTISQDTSKSQVSLTMSSVTAAESAV | |
| | YFCARASCSGGYCILDWGQGTLVTVSSASTKGPSV | |
| | FPLAPSSKSTSGGTAALGCLVKDYFPEPVTVSWNSGA | |
| | LTSGVHTFPAVLQSSGLYSLSSVVTVPSSSLGTQTYIC | |
| | NVNHKPSNTKVDKKVEPKSCDKTHTCPPCPAPELLA | |
| | GPSVFLFPPKPKDTLMISRTPEVTCVVVDVSHEDPEV | |
| | KFNWYVDGVEVHNAKTKPREEQYNSTYRVVSVLTV | |
| | LHQDWLNGKEYKCKVSNKALPAPIEKTISKAKGQPR | |
| | EPQVYTLPPSREEMTKNQVSLTCLVKGFYPSDIAVEW | |
| | ESNGQPENNYKTTPPVLDSDGSFFLYSKLTVDKSRW | |
| | QQGNVFSCSVMHEALHNHYTQKSLSLSPGK | |
| TCN032 - GAALIE | | |
| SEQ ID NO: 79 | MGWSCIILFLVATATGAHSQVQLQESGPGLVKPSETL | Heavy Chain |
| | SLTCTVSGSSISNYYWSWIRQSPGKGLEWIGFIYYGG | |
| | NTKYNPSLKSRVTISQDTSKSQVSLTMSSVTAAESAV | |
| | YFCARASCSGGYCILDWGQGTLVTVSSASTKGPSV | |
| | FPLAPSSKSTSGGTAALGCLVKDYFPEPVTVSWNSGA | |
| | LTSGVHTFPAVLQSSGLYSLSSVVTVPSSSLGTQTYIC | |
| | NVNHKPSNTKVDKKVEPKSCDKTHTCPPCPAPELLA | |
| | GPSVFLFPPKPKDTLMISRTPEVTCVVVDVSHEDPEV | |
| | KFNWYVDGVEVHNAKTKPREEQYNSTYRVVSVLTV | |
| | LHQDWLNGKEYKCKVSNKALPLPEEKTISKAKGQPR | |
| | EPQVYTLPPSREEMTKNQVSLTCLVKGFYPSDIAVEW | |
| | ESNGQPENNYKTTPPVLDSDGSFFLYSKLTVDKSRW | |
| | QQGNVFSCSVMHEALHNHYTQKSLSLSPGK | |
| 14C2 - WT | | |
| SEQ ID NO: 80 | MGWSCIILFLVATATGAHSQVQLQQSGPEVVRPGVS | Heavy Chain |
| | VKISCKGSGYIFTDYAMHWVKQSHAKSLEWIGVIST | |
| | YTGKTNYSQKFKGKATMTVDKSSSTAYMELARLTSE | |
| | DSSVYYCARRGDYDAWFAYWGQGTLVTVSSASTKG | |
| | PSVFPLAPSSKSTSGGTAALGCLVKDYFPEPVTVSWN | |
| | SGALTSGVHTFPAVLQSSGLYSLSSVVTVPSSSLGTQT | |
| | YICNVNHKPSNTKVDKKVEPKSCDKTHTCPPCPAPEL | |
| | LGGPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPAPIEKTISKAKGQ | |
| | PREPQVYTLPPSREEMTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| 14C2 - GRLR | | |
| SEQ ID NO: 81 | MGWSCIILFLVATATGAHSQVQLQQSGPEVVRPGVS | Heavy Chain |
| | VKISCKGSGYIFTDYAMHWVKQSHAKSLEWIGVIST | |
| | YTGKTNYSQKFKGKATMTVDKSSSTAYMELARLTSE | |
| | DSSVYYCARRGDYDAWFAYWGQGTLVTVSSASTKG | |
| | PSVFPLAPSSKSTSGGTAALGCLVKDYFPEPVTVSWN | |
| | SGALTSGVHTFPAVLQSSGLYSLSSVVTVPSSSLGTQT | |
| | YICNVNHKPSNTKVDKKVEPKSCDKTHTCPPCPAPEL | |
| | LRGPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKARPAPIEKTISKAKGQ | |
| | PREPQVYTLPPSREEMTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| 14C2 - GA | | |
| SEQ ID NO: 82 | MGWSCIILFLVATATGAHSQVQLQQSGPEVVRPGVS | Heavy Chain |
| | VKISCKGSGYIFTDYAMHWVKQSHAKSLEWIGVIST | |
| | YTGKTNYSQKFKGKATMTVDKSSSTAYMELARLTSE | |
| | DSSVYYCARRGDYDAWFAYWGQGTLVTVSSASTKG | |
| | PSVFPLAPSSKSTSGGTAALGCLVKDYFPEPVTVSWN | |
| | SGALTSGVHTFPAVLQSSGLYSLSSVVTVPSSSLGTQT | |
| | YICNVNHKPSNTKVDKKVEPKSCDKTHTCPPCPAPEL | |
| | LAGPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPAPIEKTISKAKGQ | |
| | PREPQVYTLPPSREEMTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| 14C2 - ALIE | | |
| SEQ ID NO: 83 | MGWSCIILFLVATATGAHSQVQLQQSGPEVVRPGVS | Heavy Chain |
| | VKISCKGSGYIFTDYAMHWVKQSHAKSLEWIGVIST | |
| | YTGKTNYSQKFKGKATMTVDKSSSTAYMELARLTSE | |
| | DSSVYYCARRGDYDAWFAYWGQGTLVTVSSASTKG | |
| | PSVFPLAPSSKSTSGGTAALGCLVKDYFPEPVTVSWN | |
| | SGALTSGVHTFPAVLQSSGLYSLSSVVTVPSSSLGTQT | |
| | YICNVNHKPSNTKVDKKVEPKSCDKTHTCPPCPAPEL | |
| | LGGPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPLPEEKTISKAKGQ | |
| | PREPQVYTLPPSREEMTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| 14C2 - GAALIE | | |
| SEQ ID NO: 84 | MGWSCIILFLVATATGAHSQVQLQQSGPEVVRPGVS | Heavy Chain |
| | VKISCKGSGYIFTDYAMHWVKQSHAKSLEWIGVIST | |
| | YTGKTNYSQKFKGKATMTVDKSSSTAYMELARLTSE | |
| | DSSVYYCARRGDYDAWFAYWGQGTLVTVSSASTKG | |
| | PSVFPLAPSSKSTSGGTAALGCLVKDYFPEPVTVSWN | |
| | SGALTSGVHTFPAVLQSSGLYSLSSVVTVPSSSLGTQT | |
| | YICNVNHKPSNTKVDKKVEPKSCDKTHTCPPCPAPEL | |
| | LAGPSVFLFPPKPKDTLMISRTPEVTCVVVDVSHEDP | |
| | EVKFNWYVDGVEVHNAKTKPREEQYNSTYRVVSVL | |
| | TVLHQDWLNGKEYKCKVSNKALPLPEEKTISKAKGQ | |
| | PREPQVYTLPPSREEMTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |

4G05 - nucleotide sequence

| | | |
|---|---|---|
| SEQ ID NO: 85 | GGATTCACCTTCAGTAGTTCCTGG | CDRH1 |
| SEQ ID NO: 86 | ATTAATAGTGGTGGGAATTTCAAA | CDRH2 |
| SEQ ID NO. 87 | GCAAGAGATCATGACTACGGTGACTACAGAGG | CDRH3 |
| | GAACGCGTATGATATC | |
| SEQ ID NO: 88 | CAGGACATTAGCAACTAT | CDRL1 |
| SEQ ID NO: 89 | GATACATCC | CDRL2 |
| SEQ ID NO: 90 | CAGCAGCTTGATAGT | CDRL3 |
| SEQ ID NO: 91 | GAGGTGCAGCTGGTGGAGTCGGGGGGAGACTT | VH |
| | AGTTCAGCCGGGGGGGTCCCTGAGACTCTCCTG | |
| | TGCAGGCTCTGGATTCACCTTCAGTAGTTCCTG | |
| | GATGCACTGGGTCCGCCAAGCTCCAGGGAAGG | |
| | GGCTGGTGTGGGTCTCACGTATTAATAGTGGTG | |
| | GGAATTTCAAAAAATACGCGGACTCCGTGAGG | |
| | GGCCGATTCACCATCTCCAGAGACAACACCAG | |
| | GAACACCCTATATCTGCATATGAGCAGTCTGAG | |
| | ACACGAGGACACGGCTCTTTATTACTGTGCAAG | |
| | AGA | |
| SEQ ID NO: 92 | GACATCCAGATGACCCAGTCTCCATCCTCCCTG | VL |
| | TCTGCATCTGTGGGAGACAGAGTCACCATCACT | |
| | TGCCAGGCGAGTCAGGACATTAGCAACTATTTC | |
| | AATTGGTATCAGCAGAAACCAGGGAAAGCCCC | |
| | TAAGCTCCTAATCTTCGATACATCCAAGTTGGA | |
| | AACAGGGGTCCCATCAAGGTTCAGTGGAAGAC | |
| | AATCTGGGACAGATTATACTTTCACCATCAGCA | |
| | GCCTGCAGCCTGAAGATATTGCAACATATTTCT | |
| | GTCAGCAGCT | |
| SEQ ID NO: 93 | ATGGGATGGTCATGTATCATCCTTTTTCTAGTA | Heavy chain |
| | GCAACTGCAACCGGTGTACATTCTGAGGTGCA | |
| | GCTGGTGGAGTCGGGGGGAGACTTAGTTCAGC | |
| | CGGGGGGGTCCCTGAGACTCTCCTGTGCAGGCT | |
| | CTGGATTCACCTTCAGTAGTTCCTGGATGCACT | |
| | GGGTCCGCCAAGCTCCAGGGAAGGGGCTGGTG | |
| | TGGGTCTCACGTATTAATAGTGGTGGGAATTTC | |
| | AAAAAATACGCGGACTCCGTGAGGGGCCGATT | |
| | CACCATCTCCAGAGACAACACCAGGAACACCC | |
| | TATATCTGCATATGAGCAGTCTGAGACACGAG | |
| | GACACGGCTCTTTATTACTGTGCAAGAGATCAT | |
| | GACTACGGTGACTACAGAGGGAACGCGTATGA | |
| | TATCTGGGGCCAAGGGACAATGGTCACCGTCTC | |
| | GAGCTCCACCAAGGGCCCATCGGTCTTCCCCCT | |
| | GGCACCCTCCTCCAAGAGCACCTCTGGGGGCA | |
| | CAGCGGCCCTGGGCTGCCTGGTCAAGGACTACT | |
| | TCCCCGAACCGGTGACGGTGTCGTGGAACTCA | |
| | GGCGCCCTGACCAGCGGCGTGCACACCTTCCCG | |
| | GCTGTCCTACAGTCCTCAGGACTCTACTCCCTC | |
| | AGCAGCGTGGTGACCGTGCCCTCCAGCAGCTTG | |
| | GGCACCCAGACCTACATCTGCAACGTGAATCA | |
| | CAAGCCCAGCAACACCAAGGTGGACAAGAGAG | |
| | TTGAGCCCAAATCTTGTGACAAAACTCACACAT | |
| | GCCCACCGTGCCCAGCACCTGAACTCCTGGGG | |
| | GGACCGTCAGTCTTCCTCTTCCCCCCAAAACCC | |
| | AAGGACACCCTCATGATCTCCCGGACCCCTGAG | |
| | GTCACATGCGTGGTGGTGGACGTGAGCCACGA | |
| | AGACCCTGAGGTCAAGTTCAACTGGTACGTGG | |
| | ACGGCGTGGAGGTGCATAATGCCAAGACAAAG | |
| | CCGCGGGAGGAGCAGTACAACAGCACGTACCG | |
| | TGTGGTCAGCGTCCTCACCGTCCTGCACCAGGA | |
| | CTGGCTGAATGGCAAGGAGTACAAGTGCAAGG | |
| | TCTCCAACAAAGCCCTCCCAGCCCCCATCGAGA | |
| | AAACCATCTCCAAAGCCAAAGGGCAGCCCCGA | |
| | GAACCACAGGTGTACACCCTGCCCCCATCCCGG | |
| | GATGAGCTGACCAAGAACCAGGTCAGCCTGAC | |
| | CTGCCTGGTCAAAGGCTTCTATCCCAGCGACAT | |
| | CGCCGTGGAGTGGGAGAGCAATGGGCAGCCGG | |
| | AGAACAACTACAAGACCACGCCTCCCGTGCTG | |
| | GACTCCGACGGCTCCTTCTTCCTCTACAGCAAG | |
| | CTCACCGTGGACAAGAGCAGGTGGCAGCAGGG | |
| | GAACGTCTTCTCATGCTCCGTGATGCATGAGGC | |
| | TCTGCACAACCACTACACGCAGAAGAGCCTCTC | |
| | CCTGTCTCCGGGTAAATGA | |
| SEQ ID NO: 94 | ATGACCCAGTCTCCATCCTCCCTGTCTGCATCT | Light chain |
| | GTGGGAGACAGAGTCACCATCACTTGCCAGGC | |
| | GAGTCAGGACATTAGCAACTATTTCAATTGGTA | |
| | TCAGCAGAAACCAGGGAAAGCCCCTAAGCTCC | |
| | TAATCTTCGATACATCCAAGTTGGAAACAGGG | |
| | GTCCCATCAAGGTTCAGTGGAAGACAATCTGG | |
| | GACAGATTATACTTTCACCATCAGCAGCCTGCA | |
| | GCCTGAAGATATTGCAACATATTTCTGTCAGCA | |
| | GCTTGATAGTTTCGGCGGAGGGACCAAGGTGG | |
| | AGCTCGAGCGAACTGTGGCTGCACCATCTGTCT | |
| | TCATCTTCCCGCCATCTGATGAGCAGTTGAAAT | |
| | CTGGAACTGCCTCTGTTGTGTGCCTGCTGAATA | |
| | ACTTCTATCCCAGAGAGGCCAAAGTACAGTGG | |
| | AAGGTGGATAACGCCCTCCAATCGGGTAACTC | |
| | CCAGGAGAGTGTCACAGAGCAGGACAGCAAGG | |
| | ACAGCACCTACAGCCTCAGCAGCACCCTGACG | |
| | CTGAGCAAAGCAGACTACGAGAAACACAAAGT | |
| | CTACGCCTGCGAAGTCACCCATCAGGGCCTGA | |
| | GCTCGCCCGTCACAAAGAGCTTCAACAGGGGA | |
| | GAGTGTTGA | |

4G05 - amino acid sequence

| | | |
|---|---|---|
| SEQ ID NO: 95 | GFTFSSSW | CDRH1 |
| SEQ ID NO: 96 | INSGGNFK | CDRH2 |
| SEQ ID NO: 97 | ARDHDYGDYRGNAYDI | CDRH3 |
| SEQ ID NO. 98 | QDISNY | CDRL1 |
| SEQ ID NO: 99 | DTS | CDRL2 |
| SEQ ID NO: 100 | QQLDS | CDRL3 |
| SEQ ID NO: 101 | EVQLVESGGDLVQPGGSLRLSCAGSGFTFSSSWM | VH |
| | HWVRQAPGKGLVWVSRINSGGNFKKYADSVRG | |
| | RFTISRDNTRNTLYLHMSSLRHEDTALYYCAR | |
| SEQ ID NO: 102 | DIQMTQSPSSLSASVGDRVTITCQASQDISNYFNW | VL |
| | YQQKPGKAPKLLIFDTSKLETGVPSRFSGRQSGTD | |
| | YTFTISSLQPEDIATYFCQQ | |
| SEQ ID NO: 103 | MGWSCIILFLVATATGVHSEVQLVESGGDLVQPG | Heavy chain |
| | GSLRLSCAGSGFTFSSSWMHWVRQAPGKGLVWV | |
| | SRINSGGNFKKYADSVRGRFTISRDNTRNTLYLH | |
| | MSSLRHEDTALYYCARDHDYGDYRGNAYDIWG | |
| | QGTMVTVSSSTKGPSVFPLAPSSKSTSGGTAALG | |
| | CLVKDYFPEPVTVSWNSGALTSGVHTFPAVLQSS | |
| | GLYSLSSVVTVPSSSLGTQTYICNVNHKPSNTKVD | |
| | KRVEPKSCDKTHTCPPCPAPELLGGPSVFLFPPKP | |
| | KDTLMISRTPEVTCVVVDVSHEDPEVKFNWYVD | |
| | GVEVHNAKTKPREEQYNSTYRVVSVLTVLHQDW | |
| | LNGKEYKCKVSNKALPAPIEKTISKAKGQPREPQ | |
| | VYTLPPSRDELTKNQVSLTCLVKGFYPSDIAVEW | |
| | ESNGQPENNYKTTPPVLDSDGSFFLYSKLTVDKS | |
| | RWQQGNVFSCSVMHEALHNHYTQKSLSLSPGK | |
| SEQ ID NO: 104 | MTQSPSSLSASVGDRVTITCQASQDISNYFNWYQ | Light chain |
| | QKPGKAPKLLIFDTSKLETGVPSRFSGRQSGTDYT | |
| | FTISSLQPEDIATYFCQQLDSFGGGTKVELERTVA | |
| | APSVFIFPPSDEQLKSGTASVVCLLNNFYPREAKV | |
| | QWKVDNALQSGNSQESVTEQDSKDSTYSLSSTLT | |
| | LSKADYEKHKVYACEVTHQGLSSPVTKSFNRGE | |
| | C | |

1A01 - nucleotide sequence

| | | |
|---|---|---|
| SEQ ID NO: 105 | GGTGGCTCCATCAGAAGTGGTATTCACTAC | CDRH1 |
| SEQ ID NO: 106 | ATCCACTACAGTGAGAATACC | CDRH2 |
| SEQ ID NO. 107 | GCGAGAGCGGCAAAAGAGTCTCTTTGTATTGGT | CDRH3 |
| | GGTAGCTGCGACTCAAACTACGAACACTACGG | |
| | TTTGGACGTC | |
| SEQ ID NO: 108 | CAGAGCATAAGGAACTAT | CDRL1 |
| SEQ ID NO. 109 | GCTGTATCC | CDRL2 |
| SEQ ID NO: 110 | CAACAGAGTTACAGCACCCTCCCGTACACT | CDRL3 |
| SEQ ID NO: 111 | CAGGTGCAGCTGCAGGAGTCGGGCCCAGGACT | VH |
| | GGTGAAGCCTTCACAGACCCTGTCCCTCACCTG | |
| | CACTGTCTCTGGTGGCTCCATCAGAAGTGGTAT | |
| | TCACTACTGGAGCTGGATCCGCCAATTCCCAGG | |
| | GAAGGGCCTGGAGTGGATTGGACTCATCCACT | |
| | ACAGTGAGAATACCCACCACAACCCGTCCCTC | |
| | AAGAGTCGAGTTGCCATGTCAGTAGACACGTCT | |
| | AAGAACCAGTTCTCCCTGACCCTGAGCTCTGTG | |
| | ACGGCCGCGGACACGGCCGTCTATTATTGTGCG | |
| | AGAG | |
| SEQ ID NO: 112 | GACATCCAGATGACCCAGTCTCCATCCTCCCTG | VL |
| | TCTGCATCTATAGGAGACAGAGTCACCATCACT | |
| | TGCCGGGCAAGTCAGAGCATAAGGAACTATTT | |
| | AAATTGGTATCAGCAGAAACCAGGCAAAGCCC | |
| | CTAAACTCGTGATCTATGCTGTATCCAATTTGC | |
| | AAAGTGGGGTCCCATCAAGGTTCAGTGGCAGT | |
| | GGATCTGGGACAGATTTCACGCTCACCATCAGC | |
| | AGTCTGCAACCTGAAGATCTTGCAACCTACTAC | |
| | TGTCAACAGAGTTACAGCACCCTCCC | |
| SEQ ID NO: 113 | ATGGGATGGTCATGTATCATCCTTTTTCTAGTA | Heavy chain |
| | GCAACTGCAACCGGTGTACATTCCCAGGTGCA | |
| | GCTGCAGGAGTCGGGCCCAGGACTGGTGAAGC | |
| | CTTCACAGACCCTGTCCCTCACCTGCACTGTCT | |
| | CTGGTGGCTCCATCAGAAGTGGTATTCACTACT | |
| | GGAGCTGGATCCGCCAATTCCCAGGGAAGGGC | |
| | CTGGAGTGGATTGGACTCATCCACTACAGTGAG | |
| | AATACCCACCACAACCCGTCCCTCAAGAGTCG | |
| | AGTTGCCATGTCAGTAGACACGTCTAAGAACC | |
| | AGTTCTCCCTGACCCTGAGCTCTGTGACGGCCG | |
| | CGGACACGGCCGTCTATTATTGTGCGAGAGCG | |
| | GCAAAAGAGTCTCTTTGTATTGGTGGTAGCTGC | |
| | GACTCAAACTACGAACACTACGGTTTGGACGTC | |
| | TGGGGCCAAGGGACCACGGTCACCGTCTCGAG | |
| | CTCCACCAAGGGCCCATCGGTCTTCCCCCTGGC | |
| | ACCCTCCTCCAAGAGCACCTCTGGGGGCACAG | |
| | CGGCCCTGGGCTGCCTGGTCAAGGACTACTTCC | |
| | CCGAACCGGTGACGGTGTCGTGGAACTCAGGC | |
| | GCCCTGACCAGCGGCGTGCACACCTTCCCGGCT | |
| | GTCCTACAGTCCTCAGGACTCTACTCCCTCAGC | |
| | AGCGTGGTGACCGTGCCCTCCAGCAGCTTGGGC | |
| | ACCCAGACCTACATCTGCAACGTGAATCACAA | |
| | GCCCAGCAACACCAAGGTGGACAAGAGAGTTG | |
| | AGCCCAAATCTTGTGACAAAACTCACACATGCC | |
| | CACCGTGCCCAGCACCTGAACTCCTGGGGGGA | |
| | CCGTCAGTCTTCCTCTTCCCCCCAAAACCCAAG | |
| | GACACCCTCATGATCTCCCGGACCCCTGAGGTC | |
| | ACATGCGTGGTGGTGGACGTGAGCCACGAAGA | |
| | CCCTGAGGTCAAGTTCAACTGGTACGTGGACG | |
| | GCGTGGAGGTGCATAATGCCAAGACAAAGCCG | |
| | CGGGAGGAGCAGTACAACAGCACGTACCGTGT | |
| | GGTCAGCGTCCTCACCGTCCTGCACCAGGACTG | |
| | GCTGAATGGCAAGGAGTACAAGTGCAAGGTCT | |
| | CCAACAAAGCCCTCCCAGCCCCCATCGAGAAA | |
| | ACCATCTCCAAAGCCAAAGGGCAGCCCCGAGA | |
| | ACCACAGGTGTACACCCTGCCCCCATCCCGGGA | |
| | TGAGCTGACCAAGAACCAGGTCAGCCTGACCT | |
| | GCCTGGTCAAAGGCTTCTATCCCAGCGACATCG | |
| | CCGTGGAGTGGGAGAGCAATGGGCAGCCGGAG | |
| | AACAACTACAAGACCACGCCTCCCGTGCTGGA | |
| | CTCCGACGGCTCCTTCTTCCTCTACAGCAAGCT | |
| | CACCGTGGACAAGAGCAGGTGGCAGCAGGGGA | |
| | ACGTCTTCTCATGCTCCGTGATGCATGAGGCTC | |
| | TGCACAACCACTACACGCAGAAGAGCCTCTCC | |
| | CTGTCTCCGGGTAAATGA | |
| SEQ ID NO: 114 | ATGGGATGGTCATGTATCATCCTTTTTCTAGTA | Light chain |
| | GCAACTGCAACCGGTGTACATTCTGACATCCAG | |
| | ATGACCCAGTCTCCATCCTCCCTGTCTGCATCT | |
| | ATAGGAGACAGAGTCACCATCACTTGCCGGGC | |
| | AAGTCAGAGCATAAGGAACTATTTAAATTGGT | |
| | ATCAGCAGAAACCAGGCAAAGCCCCTAAACTC | |
| | GTGATCTATGCTGTATCCAATTTGCAAAGTGGG | |
| | GTCCCATCAAGGTTCAGTGGCAGTGGATCTGGG | |
| | ACAGATTTCACGCTCACCATCAGCAGTCTGCAA | |
| | CCTGAAGATCTTGCAACCTACTACTGTCAACAG | |
| | AGTTACAGCACCCTCCCGTACACTTTTGGCCAG | |
| | GGGACCAAGGTGGAGATCAAACTCGAGCGGGC | |
| | TGATGCTGCACCAACTGTATCCATCTTCCCACC | |
| | ATCCAGTGAGCAGTTAACATCTGGAGGTGCCTC | |
| | AGTCGTGTGCTTCTTGAACAACTTCTACCCCAA | |
| | AGACATCAATGTCAAGTGGAAGATTGATGGCA | |
| | GTGAACGACAAAATGGCGTCCTGAACAGTTGG | |
| | ACTGATCAGGACAGCAAAGACAGCACCTACAG | |
| | CATGAGCAGCACCCTCACGTTGACCAAGGACG | |
| | AGTATGAACGACATAACAGCTATACCTGTGAG | |
| | GCCACTCACAAGACATCAACTTCACCCATTGTC | |
| | AAGAGCTTCAACAGGAATGAGTGTTGA | |

1A01 - amino acid sequence

| | | |
|---|---|---|
| SEQ ID NO: 115 | GGSIRSGIHY | CDRH1 |
| SEQ ID NO: 116 | IHYSENT | CDRH2 |
| SEQ ID NO: 117 | ARAAKESLCIGGSCDSNYEHYGLDV | CDRH3 |
| SEQ ID NO: 118 | QSIRNY | CDRL1 |
| SEQ ID NO: 119 | AVS | CDRL2 |
| SEQ ID NO: 120 | QQSYSTLPYT | CDRL3 |
| SEQ ID NO: 121 | QVQLQESGPGLVKPSQTLSLTCTVSGGSIRSGIHY | VH |
| | WSWIRQFPGKGLEWIGLIHYSENTHHNPSLKSRV | |
| | AMSVDTSKNQFSLTLSSVTAADTAVYYCAR | |
| SEQ ID NO: 122 | DIQMTQSPSSLSASIGDRVTITCRASQSIRNYLNW | VL |
| | YQQKPGKAPKLVIYAVSNLQSGVPSRFSGSGSGT | |
| | DFTLTISSLQPEDLATYYCQQSYSTL | |
| SEQ ID NO: 123 | MGWSCIILFLVATATGVHSQVQLQESGPGLVKPS | Heavy chain |
| | QTLSLTCTVSGGSIRSGIHWSWIRQFPGKGLEWI | |
| | GLIHYSENTHHNPSLKSRVAMSVDTSKNQFSLTL | |
| | SSVTAADTAVYYCARAAKESLCIGGSCDSNYEHY | |
| | GLDVWGQGTTVTVSSSTKGPSVFPLAPSSKSTSG | |
| | GTAALGCLVKDYFPEPVTVSWNSGALTSGVHTFP | |
| | AVLQSSGLYSLSSVVTVPSSSLGTQTYICNVNHKP | |
| | SNTKVDKRVEPKSCDKTHTCPPCPAPELLGGPSV | |
| | FLFPPKPKDTLMISRTPEVTCVVVDVSHEDPEVKF | |
| | NWYVDGVEVHNAKTKPREEQYNSTYRVVSVLT | |
| | VLHQDWLNGKEYKCKVSNKALPAPIEKTISKAK | |
| | GQPREPQVYTLPPSRDELTKNQVSLTCLVKGFYP | |
| | SDIAVEWESNGQPENNYKTTPPVLDSDGSFFLYS | |
| | KLTVDKSRWQQGNVFSCSVMHEALHNHYTQKS | |
| | LSLSPGK | |
| SEQ ID NO: 124 | MGWSCIILFLVATATGVHSDIQMTQSPSSLSASIG | Light chain |
| | DRVTITCRASQSIRNYLNWYQQKPGKAPKLVIYA | |
| | VSNLQSGVPSRFSGSGSGTDFTLTISSLQPEDLATY | |
| | YCQQSYSTLPYTFGQGTKVEIKLERADAAPTVSIF | |
| | PPSSEQLTSGGASVVCFLNNEYPKDINVKWKIDGS | |
| | ERQNGVLNSWTDQDSKDSTYSMSSTLTLTKDEY | |
| | ERHNSYTCEATHKTSTSPIVKSENRNEC | |
| 4G05 - WT | | |
| SEQ ID NO: | MGWSCIILFLVATATGVHSEVQLVESGGDLVQPGGS | Heavy Chain |
| 125 | LRLSCAGSGFTFSSSWMHWYRQAPGKGLVWVSRINS | |
| | GGNFKKYADSVRGRFTISRDNTRNTLYLHMSSLRHE | |
| | DTALYYCARDHDYGDYRGNAYDIWGQGTMVTVSSS | |
| | TKGPSVFPLAPSSKSTSGGTAALGCLVKDYFPEPVTV | |
| | SWNSGALTSGVHTFPAVLQSSGLYSLSSVVTVPSSSL | |
| | GTQTYICNVNHKPSNTKVDKRVEPKSCDKTHTCPPC | |
| | PAPELLGGPSVFLFPPKPKDTLMISRTPEVTCVVVDVS | |
| | HEDPEYKENWYVDGVEVHNAKTKPREEQYNSTYRY | |
| | VSVLTYLHQDWLNGKEYKCKVSNKALPAPIEKTISK | |
| | AKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPG | |
| | K | |
| 4G05 - GRLR | | |
| SEQ ID NO: | MGWSCIILFLVATATGVHSEVQLVESGGDLVQPGGS | Heavy Chain |
| 126 | LRLSCAGSGFTFSSSWMHWVRQAPGKGLVWVSRINS | |
| | GGNFKKYADSVRGRFTISRDNTRNTLYLHMSSLRHE | |
| | DTALYYCARDHDYGDYRGNAYDIWGQGTMVTVSSS | |
| | TKGPSVFPLAPSSKSTSGGTAALGCLVKDYFPEPVTV | |
| | SWNSGALTSGVHTFPAVLQSSGLYSLSSVVTVPSSSL | |
| | GTQTYICNVNHKPSNTKVDKRVEPKSCDKTHTCPPC | |
| | PAPELLRGPSVFLFPPKPKDTLMISRTPEVTCVVVDVS | |
| | HEDPEVKFNWYVDGVEVHNAKTKPREEQYNSTYRV | |
| | VSVLTVLHQDWLNGKEYKCKVSNKARPAPIEKTISK | |
| | AKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPG | |
| | K | |
| 4G05 - GA | | |
| SEQ ID NO: | MGWSCIILFLVATATGVHSEVQLVESGGDLVQPGGS | Heavy Chain |
| 127 | LRLSCAGSGFTFSSSWMHWVRQAPGKGLVWVSRINS | |
| | GGNFKKYADSVRGRFTISRDNTRNTLYLHMSSLRHE | |
| | DTALYYCARDHDYGDYRGNAYDIWGQGTMVTVSSS | |
| | TKGPSVFPLAPSSKSTSGGTAALGCLVKDYFPEPVTV | |
| | SWNSGALTSGVHTFPAVLQSSGLYSLSSVVTVPSSSL | |
| | GTQTYICNVNHKPSNTKVDKRVEPKSCDKTHTCPPC | |
| | PAPELLAGPSVFLFPPKPKDTLMISRTPEVTCVVVDVS | |
| | HEDPEVKFNWYVDGVEVHNAKTKPREEQYNSTYRV | |
| | VSVLTVLHQDWLNGKEYKCKVSNKALPAPIEKTISK | |
| | AKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPG | |
| | K | |
| 4G05 - ALIE | | |
| SEQ ID NO: | MGWSCIILFLVATATGVHSEVQLVESGGDLVQPGGS | Heavy Chain |
| 128 | LRLSCAGSGFTFSSSWMHWVRQAPGKGLVWVSRINS | |
| | GGNFKKYADSVRGRFTISRDNTRNTLYLHMSSLRHE | |
| | DTALYYCARDHDYGDYRGNAYDIWGQGTMVTVSSS | |
| | TKGPSVFPLAPSSKSTSGGTAALGCLVKDYFPEPVTV | |
| | SWNSGALTSGVHTFPAVLQSSGLYSLSSVVTVPSSSL | |
| | GTQTYICNVNHKPSNTKVDKRVEPKSCDKTHTCPPC | |
| | PAPELLGGPSVFLFPPKPKDTLMISRTPEVTCVVVDVS | |
| | HEDPEVKFNWYVDGVEVHNAKTKPREEQYNSTYRV | |
| | VSVLTVLHQDWLNGKEYKCKVSNKALPLPEEKTISK | |
| | AKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPG | |
| | K | |
| 4G05 - GAALIE | | |
| SEQ ID NO: | MGWSCIILFLVATATGVHSEVQLVESGGDLVQPGGS | Heavy Chain |
| 129 | LRLSCAGSGFTFSSSWMHWVRQAPGKGLVWVSRINS | |
| | GGNFKKYADSVRGRFTISRDNTRNTLYLHMSSLRHE | |
| | DTALYYCARDHDYGDYRGNAYDIWGQGTMVTVSSS | |
| | TKGPSVFPLAPSSKSTSGGTAALGCLVKDYFPEPVTV | |
| | SWNSGALTSGVHTFPAVLQSSGLYSLSSVVTVPSSSL | |
| | GTQTYICNVNHKPSNTKVDKRVEPKSCDKTHTCPPC | |
| | PAPELLAGPSVFLFPPKPKDTLMISRTPEVTCVVVDVS | |
| | HEDPEVKFNWYVDGVEVHNAKTKPREEQYNSTYRV | |
| | VSVLTVLHQDWLNGKEYKCKVSNKALPLPEEKTISK | |
| | AKGQPREPQVYTLPPSREEMTKNQVSLTCLVKGFYP | |
| | SDIAVEWESNGQPENNYKTTPPVLDSDGSFFLYSKLT | |
| | VDKSRWQQGNVFSCSVMHEALHNHYTQKSLSLSPG | |
| | K | |
| 1A01 - WT | | |
| SEQ ID NO: | MGWSCIILFLVATATGVHSQVQLQESGPGLVKPSQTL | Heavy Chain |
| 130 | SLTCTVSGGSIRSGIHYWSWIRQFPGKGLEWIGLIHYS | |
| | ENTHHNPSLKSRVAMSVDTSKNQFSLTLSSVTAADT | |
| | AVYYCARAAKESLCIGGSCDSNYEHYGLDVWGQGT | |
| | TVTVSSSTKGPSVFPLAPSSKSTSGGTAALGCLVKDY | |
| | FPEPVTVSWNSGALTSGVHTFPAVLQSSGLYSLSSVV | |
| | TVPSSSLGTQTYICNVNHKPSNTKVDKRVEPKSCDKT | |
| | HTCPPCPAPELLGGPSVFLFPPKPKDTLMISRTPEVTC | |
| | VVVDVSHEDPEVKFNWYVDGVEVHNAKTKPREEQY | |
| | NSTYRVVSVLTVLHQDWLNGKEYKCKVSNKALPAPI | |
| | EKTISKAKGQPREPQVYTLPPSREEMTKNQVSLTCLV | |
| | KGFYPSDIAVEWESNGQPENNYKTTPPVLDSDGSFFL | |
| | YSKLTVDKSRWQQGNVFSCSVMHEALHNHYTQKSL | |
| | SLSPGK | |
| 1A01 - GRLR | | |
| SEQ ID NO: | MGWSCIILFLVATATGVHSQVQLQESGPGLVKPSQTL | Heavy Chain |
| 131 | SLTCTVSGGSIRSGIHYWSWIRQFPGKGLEWIGLIHYS | |
| | ENTHHNPSLKSRVAMSVDTSKNQFSLTLSSVTAADT | |
| | AVYYCARAAKESLCIGGSCDSNYEHYGLDVWGQGT | |
| | TVTVSSSTKGPSVFPLAPSSKSTSGGTAALGCLVKDY | |
| | FPEPVTVSWNSGALTSGVHTFPAVLQSSGLYSLSSVV | |
| | TVPSSSLGTQTYICNVNHKPSNTKVDKRVEPKSCDKT | |
| | HTCPPCPAPELLRGPSVFLFPPKPKDTLMISRTPEVTC | |
| | VVVDVSHEDPEVKFNWYVDGVEVHNAKTKPREEQY | |
| | NSTYRVVSVLTVLHQDWLNGKEYKCKVSNKARPAPI | |
| | EKTISKAKGQPREPQVYTLPPSREEMTKNQVSLTCLV | |
| | KGFYPSDIAVEWESNGQPENNYKTTPPVLDSDGSFFL | |
| | YSKLTVDKSRWQQGNVFSCSVMHEALHNHYTQKSL | |
| | SLSPGK | |
| 1A01 - GA | | |
| SEQ ID NO: | MGWSCIILFLVATATGVHSQVQLQESGPGLVKPSQTL | Heavy Chain |
| 132 | SLTCTVSGGSIRSGIHYWSWIRQFPGKGLEWIGLIHYS | |
| | ENTHHNPSLKSRVAMSVDTSKNQFSLTLSSVTAADT | |
| | AVYYCARAAKESLCIGGSCDSNYEHYGLDVWGQGT | |
| | TVTVSSSTKGPSVFPLAPSSKSTSGGTAALGCLVKDY | |
| | FPEPVTVSWNSGALTSGVHTFPAVLQSSGLYSLSSVV | |
| | TVPSSSLGTQTYICNVNHKPSNTKVDKRVEPKSCDKT | |
| | HTCPPCPAPELLAGPSVFLFPPKPKDTLMISRTPEVTC | |
| | VVVDVSHEDPEVKFNWYVDGVEVHNAKTKPREEQY | |
| | NSTYRVVSVLTVLHQDWLNGKEYKCKVSNKALPAPI | |
| | EKTISKAKGQPREPQVYTLPPSREEMTKNQVSLTCLV | |
| | KGFYPSDIAVEWESNGQPENNYKTTPPVLDSDGSFFL | |
| | YSKLTVDKSRWQQGNVFSCSVMHEALHNHYTQKSL | |
| | SLSPGK | |
| 1A01 - ALIE | | |
| SEQ ID NO: | MGWSCIILFLVATATGVHSQVQLQESGPGLVKPSQTL | Heavy Chain |
| 133 | SLTCTVSGGSIRSGIHYWSWIRQFPGKGLEWIGLIHYS | |
| | ENTHHNPSLKSRVAMSVDTSKNQFSLTLSSVTAADT | |
| | AVYYCARAAKESLCIGGSCDSNYEHYGLDVWGQGT | |
| | TVTVSSSTKGPSVFPLAPSSKSTSGGTAALGCLVKDY | |
| | FPEPVTVSWNSGALTSGVHTFPAVLQSSGLYSLSSVV | |
| | TVPSSSLGTQTYICNVNHKPSNTKVDKRVEPKSCDKT | |
| | HTCPPCPAPELLGGPSVFLFPPKPKDTLMISRTPEVTC | |
| | VVVDVSHEDPEVKFNWYVDGVEVHNAKTKPREEQY | |
| | NSTYRVVSVLTVLHQDWLNGKEYKCKVSNKALPLP | |
| | EEKTISKAKGQPREPQVYTLPPSREEMTKNQVSLTCL | |
| | VKGFYPSDIAVEWESNGQPENNYKTTPPVLDSDGSFF | |
| | LYSKLTVDKSRWQQGNVFSCSVMHEALHNHYTQKS | |
| | LSLSPGK | |
| 1A01 - GAALIE | | |
| SEQ ID NO: | MGWSCIILFLVATATGVHSQVQLQESGPGLVKPSQTL | Heavy Chain |
| 134 | SLTCTVSGGSIRSGIHYWSWIRQFPGKGLEWIGLIHYS | |
| | ENTHHNPSLKSRVAMSVDTSKNQFSLTLSSVTAADT | |
| | AVYYCARAAKESLCIGGSCDSNYEHYGLDVWGQGT | |
| | TVTVSSSTKGPSVFPLAPSSKSTSGGTAALGCLVKDY | |
| | FPEPVTVSWNSGALTSGVHTFPAVLQSSGLYSLSSVV | |
| | TVPSSSLGTQTYICNVNHKPSNTKVDKRVEPKSCDKT | |
| | HTCPPCPAPELLAGPSVFLFPPKPKDTLMISRTPEVTC | |
| | VVVDVSHEDPEVKFNWYVDGVEVHNAKTKPREEQY | |
| | NSTYRVVSVLTVLHQDWLNGKEYKCKVSNKALPLP | |
| | EEKTISKAKGQPREPQVYTLPPSREEMTKNQVSLTCL | |
| | VKGFYPSDIAVEWESNGQPENNYKTTPPVLDSDGSFF | |
| | LYSKLTVDKSRWQQGNVFSCSVMHEALHNHYTQKS | |
| | LSLSPGK | |

What is claimed is:

1. An isolated Fc receptor-dependent antibody or antigen binding portion thereof capable of activating dendritic cell maturation.

2. An isolated Fc receptor-dependent antibody or antigen binding portion thereof capable of inducing a protective CD8 response.

3. The antibody or antigen binding portion thereof of claim 1 wherein the antibody or antigen binding portion thereof binds specifically to a viral antigen.

4. The antibody or antigen binding portion thereof of claim 3, wherein the viral antigen comprises an influenza virus antigen comprising hemagglutinin (HA) or neuraminidase (NA).

5. The antibody or antigen binding portion thereof of claim 1, wherein the antibody or antigen binding portion thereof comprises (i) a heavy chain having a G236A mutation in a constant region thereof and (ii) an Fc region, wherein the Fc region activates FcγRIIa.

6. The antibody or antigen binding portion thereof of claim 5, further comprising mutations A330L and I332E in the constant region of the heavy chain.

7. The antibody or antigen binding portion thereof of claim 1, comprising:
(i) the heavy chain CDR1, CDR2, and CDR3 sequences as set forth in SEQ ID NO: 1, SEQ ID NO: 2, and SEQ ID NO: 3, respectively; and the light chain CDR1, CDR2, and CDR3 sequences as set forth in: SEQ ID NO: 4, SEQ ID NO: 5, and SEQ ID NO: 6, respectively;
(ii) the heavy chain CDR1, CDR2, and CDR3 sequences as set forth in SEQ ID NO: 26, SEQ ID NO: 27, and SEQ ID NO: 28, respectively; and the light chain CDR1, CDR2, and CDR3 sequences as set forth in: SEQ ID NO: 29, SEQ ID NO: 30, and SEQ ID NO: 31, respectively;
(iii) the heavy chain CDR1, CDR2, and CDR3 sequences as set forth in SEQ ID NO: 36, SEQ ID NO: 37, and SEQ ID NO: 38, respectively; and the light chain CDR1, CDR2, and CDR3 sequences as set forth in: SEQ ID NO: 39, SEQ ID NO: 40, and SEQ ID NO: 41, respectively;
(iv) the heavy chain CDR1, CDR2, and CDR3 sequences as set forth in SEQ ID NO: 46, SEQ ID NO: 47, and SEQ ID NO: 48, respectively; and the light chain CDR1, CDR2, and CDR3 sequences as set forth in: SEQ ID NO: 49, SEQ ID NO: 50, and SEQ ID NO: 51, respectively; or
(v) the heavy chain CDR1, CDR2, and CDR3 sequences as set forth in SEQ ID NO: 56, SEQ ID NO: 57, and SEQ ID NO: 58, respectively; and the light chain CDR1, CDR2, and CDR3 sequences as set forth in: SEQ ID NO: 59, SEQ ID NO: 60, and SEQ ID NO: 61, respectively.

8. The antibody or antigen binding portion thereof of claim 1, wherein the antibody or antigen binding portion thereof does not comprise the mutation S239D in the constant region of the heavy chain.

9. The antibody or antigen binding portion thereof of claim 1, wherein the antibody or antigen binding portion thereof comprises a half-life increasing mutation in the constant region of the heavy chain.

10. The antibody or antigen binding portion thereof of claim 9, wherein the antibody or antigen binding portion thereof comprises the mutations M428L and N434S in the constant region of the heavy chain.

11. The antibody or antigen binding portion thereof of claim 1, comprising: the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations M428L and N434S in the constant region of the heavy chain.

12. The antibody or antigen binding portion thereof of claim 1, wherein the antibody or antigen binding portion thereof binds to HA of an influenza A virus.

13. The antibody or antigen binding portion thereof of claim 1, wherein the antibody or antigen binding portion thereof neutralizes infection with an influenza A virus.

14. The antibody or antigen binding portion thereof of claim 1, wherein the antibody or antigen binding portion thereof is afucosylated.

15. The antibody or antigen binding portion thereof of claim 1, wherein the antibody or antigen binding portion thereof does not comprise the mutations G236R and L328R in the constant regions of the heavy chain.

16. The antibody or antigen binding portion thereof of claim 1, wherein the antibody or antigen binding portion thereof does not comprise the mutations G237D, P238D, H268D, P271G, and A330R in the constant regions of the heavy chain.

17. The antibody or antigen binding portion thereof of claim 1, wherein the antibody or antigen binding portion thereof is a human antibody.

18. The antibody or antigen binding portion thereof of claim 1, wherein the antibody or antigen binding portion thereof is a monoclonal antibody.

19. The antibody or antigen binding portion thereof of claim 1, wherein the antibody or antigen binding portion thereof is of the IgG type.

20. The antibody or antigen binding portion thereof of claim 19, wherein the antibody or antigen binding portion thereof is of the IgG1 type.

21. The antibody or antigen binding portion thereof of claim 1, wherein the light chain of the antibody or antigen binding portion thereof is a kappa light chain.

22. The antibody or antigen binding portion thereof of claim 1, wherein the antibody or antigen binding portion thereof comprises:
(i) a heavy chain variable region comprising an amino acid sequence having at least 75% identity to SEQ ID NO: 7 and a light chain variable region comprising the amino acid sequence having at least 75% identity to SEQ ID NO: 8;
(ii) a heavy chain variable region comprising an amino acid sequence having at least 75% identity to SEQ ID NO: 32 and a light chain variable region comprising the amino acid sequence having at least 75% identity to SEQ ID NO: 33;
(iii) a heavy chain variable region comprising an amino acid sequence having at least 75% identity to SEQ ID NO: 42 and a light chain variable region comprising the amino acid sequence having at least 75% identity to SEQ ID NO: 43;
(iv) a heavy chain variable region comprising an amino acid sequence having at least 75% identity to SEQ ID NO: 52 and a light chain variable region comprising the amino acid sequence having at least 75% identity to SEQ ID NO: 53; or
(v) a heavy chain variable region comprising an amino acid sequence having at least 75% identity to SEQ ID NO: 62 and a light chain variable region comprising the amino acid sequence having at least 75% identity to SEQ ID NO: 63.

23. The antibody or antigen binding portion thereof of claim 1, wherein the antibody or antigen binding portion thereof comprises:
(i) the heavy chain CDR1, CDR2, and CDR3 sequences as set forth in SEQ ID NO: 1, SEQ ID NO: 2, and SEQ ID NO: 3, respectively, and the light chain CDR1, CDR2, and CDR3 sequences as set forth in: SEQ ID NO: 4, SEQ ID NO: 5, and SEQ ID NO: 6, respectively; and a heavy chain variable region comprising an amino acid sequence having at least 75% identity to SEQ ID NO: 7 and a light chain variable region comprising the amino acid sequence having at least 75% identity to SEQ ID NO: 8;
(ii) the heavy chain CDR1, CDR2, and CDR3 sequences as set forth in SEQ ID NO: 26, SEQ ID NO: 27, and SEQ ID NO: 28, respectively, and the light chain CDR1, CDR2, and CDR3 sequences as set forth in: SEQ ID NO: 29, SEQ ID NO: 30, and SEQ ID NO: 31, respectively; and a heavy chain variable region comprising an amino acid sequence having at least 75% identity to SEQ ID NO: 32 and a light chain variable region comprising the amino acid sequence having at least 75% identity to SEQ ID NO: 33;
(iii) the heavy chain CDR1, CDR2, and CDR3 sequences as set forth in SEQ ID NO: 36, SEQ ID NO: 37, and SEQ ID NO: 38, respectively, and the light chain CDR1, CDR2, and CDR3 sequences as set forth in: SEQ ID NO: 39, SEQ ID NO: 40, and SEQ ID NO: 41, respectively; and a heavy chain variable region comprising an amino acid sequence having at least 75% identity to SEQ ID NO: 42 and a light chain variable region comprising the amino acid sequence having at least 75% identity to SEQ ID NO: 43;
(iv) the heavy chain CDR1, CDR2, and CDR3 sequences as set forth in SEQ ID NO: 46, SEQ ID NO: 47, and SEQ ID NO: 48, respectively, and the light chain CDR1, CDR2, and CDR3 sequences as set forth in: SEQ ID NO: 49, SEQ ID NO: 50, and SEQ ID NO: 51, respectively; and a heavy chain variable region comprising an amino acid sequence having at least 75% identity to SEQ ID NO: 52 and a light chain variable region comprising the amino acid sequence having at least 75% identity to SEQ ID NO: 53; or
(v) the heavy chain CDR1, CDR2, and CDR3 sequences as set forth in SEQ ID NO: 56, SEQ ID NO: 57, and SEQ ID NO: 58, respectively, and the light chain CDR1, CDR2, and CDR3 sequences as set forth in: SEQ ID NO: 59, SEQ ID NO: 60, and SEQ ID NO: 61, respectively; and a heavy chain variable region comprising an amino acid sequence having at least 75% identity to SEQ ID NO: 62 and a light chain variable region comprising the amino acid sequence having at least 75% identity to SEQ ID NO: 63.

24. The antibody or antigen binding portion thereof of claim 1, wherein the antibody or antigen binding portion thereof comprises:
(i) the heavy chain CDR1, CDR2, and CDR3 sequences as set forth in SEQ ID NO: 1, SEQ ID NO: 2, and SEQ ID NO: 3, respectively, and the light chain CDR1, CDR2, and CDR3 sequences as set forth in: SEQ ID NO: 4, SEQ ID NO: 5, and SEQ ID NO: 6, respectively; and a heavy chain variable region comprising an amino acid sequence as set forth in SEQ ID NO: 7 and a light chain variable region comprising the amino acid sequence as set forth in SEQ ID NO: 8;
(ii) the heavy chain CDR1, CDR2, and CDR3 sequences as set forth in SEQ ID NO: 26, SEQ ID NO: 27, and SEQ ID NO: 28, respectively, and the light chain CDR1, CDR2, and CDR3 sequences as set forth in: SEQ ID NO: 29, SEQ ID NO: 30, and SEQ ID NO: 31, respectively; and a heavy chain variable region comprising an amino acid sequence set forth in SEQ ID NO: 32 and a light chain variable region comprising the amino acid sequence set forth in SEQ ID NO: 33;
(iii) the heavy chain CDR1, CDR2, and CDR3 sequences as set forth in SEQ ID NO: 36, SEQ ID NO: 37, and SEQ ID NO: 38, respectively, and the light chain CDR1, CDR2, and CDR3 sequences as set forth in: SEQ ID NO: 39, SEQ ID NO: 40, and SEQ ID NO: 41, respectively; and a heavy chain variable region comprising an amino acid sequence set forth in SEQ ID NO: 42 and a light chain variable region comprising the amino acid sequence set forth in SEQ ID NO: 43;
(iv) the heavy chain CDR1, CDR2, and CDR3 sequences as set forth in SEQ ID NO: 46, SEQ ID NO: 47, and SEQ ID NO: 48, respectively, and the light chain CDR1, CDR2, and CDR3 sequences as set forth in: SEQ ID NO: 49, SEQ ID NO: 50, and SEQ ID NO: 51, respectively; and a heavy chain variable region comprising an amino acid sequence set forth in SEQ ID NO: 52 and a light chain variable region comprising the amino acid sequence set forth in SEQ ID NO: 53; or
(v) the heavy chain CDR1, CDR2, and CDR3 sequences as set forth in SEQ ID NO: 56, SEQ ID NO: 57, and SEQ ID NO: 58, respectively, and the light chain CDR1, CDR2, and CDR3 sequences as set forth in: SEQ ID NO: 59, SEQ ID NO: 60, and SEQ ID NO: 61, respectively; and a heavy chain variable region comprising an amino acid sequence set forth in SEQ ID NO: 62 and a light chain variable region comprising the amino acid sequence set forth in SEQ ID NO: 63.

25. The antibody or antigen binding portion thereof of claim 1, wherein the CH2 region of the antibody or antigen binding portion thereof does not comprise any further mutation in addition to G236A.

26. The antibody or antigen binding portion thereof of claim 1, wherein the CH2 region of the antibody or antigen binding portion thereof does not comprise any further mutation in addition to G236A, A330L, and 1332E.

27. The antibody or antigen binding portion thereof of claim 11, wherein the CH3 region of the antibody or antigen binding portion thereof does not comprise any further mutation in addition to M428L and N434S.

28. The antibody or antigen binding portion thereof of claim 1, wherein the Fc region of the antibody or antigen binding portion thereof does not comprise any further mutation in addition to G236A, A330L, and I332E and, optionally, M428L and N434S.

29. The antibody or antigen binding portion thereof of claim 12, wherein the Fc region of the antibody or antigen binding portion thereof does not comprise any further mutation in addition to M428L and N434S.

30. The antibody or antigen binding portion thereof of claim 1, wherein the antibody or antigen binding portion thereof comprises a light chain comprising an amino acid sequence as set forth in SEQ ID NO: 10 and a heavy chain comprising an amino acid sequence as set forth in SEQ ID NOs: 9, 13, 14, 18, or 19.

31. The antibody or antigen binding portion thereof of claim 1, wherein the antibody or antigen binding portion thereof comprises a light chain comprising an amino acid sequence as set forth in SEQ ID NO: 35 and a heavy chain comprising an amino acid sequence as set forth in SEQ ID NOs: 66, 68, 69 or 70.

32. The antibody or antigen binding portion thereof of claim 1, wherein the antibody or antigen binding portion thereof comprises a light chain comprising an amino acid sequence as set forth in SEQ ID NO: 45 and a heavy chain comprising an amino acid sequence as set forth in SEQ ID NOs: 73, 74 or 75.

33. The antibody or antigen binding portion thereof of claim 1, wherein the antibody or antigen binding portion thereof comprises a light chain comprising an amino acid sequence as set forth in SEQ ID NO: 55 and a heavy chain comprising an amino acid sequence as set forth in SEQ ID NOs: 77, 78 or 79.

34. The antibody or antigen binding portion thereof of claim 1, wherein the antibody or antigen binding portion thereof comprises a light chain comprising an amino acid sequence as set forth in SEQ ID NO: 65 and a heavy chain comprising an amino acid sequence as set forth in SEQ ID NOs: 81, 82, 83 or 84.

35. The antibody or antigen binding portion thereof of claim 1 for use in prophylaxis or treatment of infection with influenza A virus.

36. The antibody or antigen binding portion thereof for use according to claim 35, wherein the antibody or antigen binding portion thereof is administered prophylcatically or therapeutically.

37. A nucleic acid molecule comprising a polynucleotide encoding the antibody or antigen binding portion thereof of claim 1.

38. A vector comprising the nucleic acid molecule of claim 37.

39. A cell expressing the antibody or antigen binding portion thereof of claim 1.

40. A pharmaceutical composition comprising the antibody or antigen binding portion thereof of claim 1 and, optionally, a pharmaceutically acceptable diluent or carrier.

41. Use of the antibody or antigen binding portion thereof of claim 1 in the manufacture of a medicament for prophylaxis, treatment or attenuation of influenza A virus infection.

42. The antibody or antigen binding portion thereof of claim 1, the nucleic acid of claim 37, the vector of claim 38, or the cell of claim 39, or the pharmaceutical composition of claim 40 for use in prophylaxis or treatment of infection with influenza A virus.

43. The antibody or antigen binding portion thereof, the nucleic acid, the vector, the cell, or the pharmaceutical composition for use according to claim 42, wherein the antibody or antigen binding portion thereof, the nucleic acid, the vector, the cell, or the pharmaceutical composition is administered prophylactically or therapeutically.

44. A method of reducing influenza A virus infection, or lowering the risk of influenza A virus infection, comprising: administering to a subject in need thereof, a therapeutically effective amount of the antibody or antigen binding portion thereof of claim 1.

45. The method of claim 44, wherein the antibody or antigen binding portion thereof is administered prophylactically or therapeutically.
